SEQUENCE LISTING (87 sequences)

SEQ ID NO: 1 - skipped

SEQ ID NO: 2 - protein, 4 aa, synthetic construct
  REPEAT 1..4: note = Repeat from 1 to 8 times
  REGION 1..4: note = linker
GGGS

SEQ ID NO: 3 - protein, 5 aa, synthetic construct
  REPEAT 1..5: note = Repeat from 1 to 8 times
  REGION 1..5: note = linker
GGGGS

SEQ ID NO: 4 - protein, 6 aa, synthetic construct
  REPEAT 1..6: note = Repeat from 1 to 8 times
  REGION 1..6: note = linker
GGGGGS

SEQ ID NO: 5 - protein, 9 aa, synthetic construct
  VARIANT 3: note = X is any amino acid
  VARIANT 4: note = X is any amino acid
  VARIANT 6: note = X is Serine or Cysteine
  VARIANT 8: note = X is any amino acid
  VARIANT 1: note = M is either present or, when located at the N terminal extremity, can be removed in vivo by post-translation processing
  REGION 1..9: note = sub-membrane targeting domain
MGXXKXKXK

SEQ ID NO: 6 - protein, 16 aa, synthetic construct
  VARIANT 3: note = X is any amino acid
  VARIANT 4: note = X is any amino acid
  VARIANT 6: note = X is Serine or Cysteine
  VARIANT 8: note = X is any amino acid
  VARIANT 10: note = X is any amino acid
  VARIANT 11: note = X is any amino acid
  VARIANT 12: note = X is any amino acid
  VARIANT 13: note = X is any amino acid
  VARIANT 1: note = M is either present or, when located at the N terminal extremity, can be removed in vivo by post-translation processing
  REGION 1..16: note = sub-membrane targeting domain
MGXXKXKXKXXXXRRR

SEQ ID NO: 7 - protein, 16 aa, synthetic construct
  VARIANT 1: note = M is either present or, when located at the N terminal extremity, can be removed in vivo by post-translation processing
  REGION 1..16: note = sub-membrane targeting domain
MGSSKSKPKDPSQRRR

SEQ ID NO: 8 - protein, 19 aa, synthetic construct
  VARIANT 1: note = M is either present or, when located at the N terminal extremity, can be removed in vivo by post-translation processing
  REGION 1..19: note = sub-membrane targeting domain
MGSSKSKPKDPSQRRRKSR

SEQ ID NO: 9 - protein, 23 aa, synthetic construct
  VARIANT 1: note = M is either present or, when located at the N terminal extremity, can be removed in vivo by post-translation processing
  REGION 1..23: note = sub-membrane targeting domain
MGSSKSKPKDPSQRRRKSRGPGG

SEQ ID NO: 10 - protein, 4 aa, synthetic construct
  REGION 1..4: note = Pilot peptide
YINL

SEQ ID NO: 11 - protein, 4 aa, synthetic construct
  REGION 1..4: note = Pilot peptide
YSHL

SEQ ID NO: 12 - protein, 4 aa, synthetic construct
  REGION 1..4: note = Pilot peptide
YINF

SEQ ID NO: 13 - protein, 4 aa, synthetic construct
  REGION 1..4: note = Pilot peptide
YSHF

SEQ ID NO: 14 - skipped

SEQ ID NO: 15 - protein, 5 aa, synthetic construct
  REGION 1..5: note = Pilot peptide
DYINL

SEQ ID NO: 16 - skipped

SEQ ID NO: 17 - protein, 5 aa, synthetic construct
  REGION 1..5: note = Pilot peptide
DYINF

SEQ ID NO: 18 - protein, 4 aa, synthetic construct
  REGION 1..4: note = Pilot peptide
PSAP

SEQ ID NO: 19 - protein, 4 aa, synthetic construct
  REGION 1..4: note = Pilot peptide
PTAP

SEQ ID NO: 20 - protein, 40 aa, synthetic construct
  REGION 1..40: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 7: note = X represents any amino acid residue
  VARIANT 8: note = X represents any amino acid residue
  VARIANT 10: note = X represents any amino acid residue
  VARIANT 11: note = X represents any amino acid residue
  VARIANT 13: note = X represents any amino acid residue
  VARIANT 15: note = X represents any amino acid residue
  VARIANT 17: note = X represents any amino acid residue
  VARIANT 18: note = X represents any amino acid residue
  VARIANT 20: note = X represents any amino acid residue
  VARIANT 22: note = X represents any amino acid residue
  VARIANT 23: note = X represents any amino acid residue
  VARIANT 26: note = X represents any amino acid residue
  VARIANT 28: note = X represents any amino acid residue
  VARIANT 29: note = X represents any amino acid residue
  VARIANT 31: note = X represents any amino acid residue
  VARIANT 33: note = X represents any amino acid residue
  VARIANT 34: note = X represents any amino acid residue
  VARIANT 38: note = X represents any amino acid residue
  VARIANT 39: note = X represents any amino acid residue
PXXPXXXXPXXPXSXYXXLXPXXPEXYXXLXPXXPDYXXL

SEQ ID NO: 21 - protein, 37 aa, synthetic construct
  REGION 1..37: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any amino acid residue
  VARIANT 8: note = X represents any amino acid residue
  VARIANT 10: note = X represents any one or several amino acid residue(s)
  VARIANT 12: note = X represents any amino acid residue
  VARIANT 14: note = X represents any amino acid residue
  VARIANT 15: note = X represents any amino acid residue
  VARIANT 17: note = X represents any one or several amino acid residue(s)
  VARIANT 19: note = X represents any amino acid residue
  VARIANT 20: note = X represents any amino acid residue
  VARIANT 23: note = X represents any one or several amino acid residue(s)
  VARIANT 25: note = X represents any amino acid residue
  VARIANT 26: note = X represents any amino acid residue
  VARIANT 28: note = X represents any one or several amino acid residue(s)
  VARIANT 30: note = X represents any amino acid residue
  VARIANT 31: note = X represents any amino acid residue
  VARIANT 35: note = X represents any amino acid residue
  VARIANT 36: note = X represents any amino acid residue
PXXPXPXXPXSXYXXLXPXXPEXYXXLXPXXPDYXXL

SEQ ID NO: 22 - protein, 44 aa, synthetic construct
  REGION 1..44: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 7: note = X represents any amino acid residue
  VARIANT 8: note = X represents any amino acid residue
  VARIANT 10: note = X represents any amino acid residue
  VARIANT 11: note = X represents any amino acid residue
  VARIANT 13: note = X represents any amino acid residue
  VARIANT 15: note = X represents any amino acid residue
  VARIANT 17: note = X represents any amino acid residue
  VARIANT 18: note = X represents any amino acid residue
  VARIANT 20: note = X represents any amino acid residue
  VARIANT 22: note = X represents any amino acid residue
  VARIANT 23: note = X represents any amino acid residue
  VARIANT 26: note = X represents any amino acid residue
  VARIANT 28: note = X represents any amino acid residue
  VARIANT 29: note = X represents any amino acid residue
  VARIANT 31: note = X represents any amino acid residue
  VARIANT 33: note = X represents any amino acid residue
  VARIANT 34: note = X represents any amino acid residue
  VARIANT 38: note = X represents any amino acid residue
  VARIANT 39: note = X represents any amino acid residue
  VARIANT 41: note = X represents any amino acid residue
  VARIANT 42: note = X represents any amino acid residue
  VARIANT 43: note = X represents any amino acid residue
  VARIANT 44: note = X represents any amino acid residue
PXXPXXXXPXXPXSXYXXLXPXXPEXYXXLXPXXPDYXXLXXXX

SEQ ID NO: 23 - protein, 41 aa, synthetic construct
  REGION 1..41: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any amino acid residue
  VARIANT 8: note = X represents any amino acid residue
  VARIANT 10: note = X represents any one or several amino acid residue(s)
  VARIANT 12: note = X represents any amino acid residue
  VARIANT 15: note = X represents any amino acid residue
  VARIANT 17: note = X represents any one or several amino acid residue(s)
  VARIANT 19: note = X represents any amino acid residue
  VARIANT 20: note = X represents any amino acid residue
  VARIANT 23: note = X represents any one or several amino acid residue(s)
  VARIANT 25: note = X represents any amino acid residue
  VARIANT 26: note = X represents any amino acid residue
  VARIANT 28: note = X represents any one or several amino acid residue(s)
  VARIANT 30: note = X represents any amino acid residue
  VARIANT 31: note = X represents any amino acid residue
  VARIANT 35: note = X represents any amino acid residue
  VARIANT 36: note = X represents any amino acid residue
  VARIANT 38: note = X represents any amino acid residue
  VARIANT 39: note = X represents any amino acid residue
  VARIANT 40: note = X represents any amino acid residue
  VARIANT 41: note = X represents any amino acid residue
  VARIANT 14: note = X represents any amino acid residue
PXXPXPXXPXSXYXXLXPXXPEXYXXLXPXXPDYXXLXXXX

SEQ ID NO: 24 - protein, 20 aa, synthetic construct
  REGION 1..20: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 7: note = X represents any amino acid residue
  VARIANT 8: note = X represents any amino acid residue
  VARIANT 9: note = X represents any amino acid residue
  VARIANT 10: note = X represents any amino acid residue
  VARIANT 11: note = X represents any amino acid residue
  VARIANT 12: note = X represents any amino acid residue
  VARIANT 13: note = X represents any amino acid residue
  VARIANT 14: note = X represents any amino acid residue
  VARIANT 15: note = X represents any amino acid residue
  VARIANT 16: note = X represents any amino acid residue
  VARIANT 18: note = X represents any amino acid residue
  VARIANT 19: note = X represents any amino acid residue
PXXPXXXXXXXXXXXXYXXL

SEQ ID NO: 25 - protein, 20 aa, synthetic construct
  REGION 1..20: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 7: note = X represents any amino acid residue
  VARIANT 8: note = X represents any amino acid residue
  VARIANT 9: note = X represents any amino acid residue
  VARIANT 10: note = X represents any amino acid residue
  VARIANT 11: note = X represents any amino acid residue
  VARIANT 12: note = X represents any amino acid residue
  VARIANT 13: note = X represents any amino acid residue
  VARIANT 14: note = X represents any amino acid residue
  VARIANT 15: note = X represents any amino acid residue
  VARIANT 18: note = X represents any amino acid residue
  VARIANT 19: note = X represents any amino acid residue
PXXPXXXXXXXXXXXDYXXL

SEQ ID NO: 26 - protein, 20 aa, synthetic construct
  REGION 1..20: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 8: note = X represents any amino acid residue
  VARIANT 9: note = X represents any amino acid residue
  VARIANT 10: note = X represents any amino acid residue
  VARIANT 11: note = X represents any amino acid residue
  VARIANT 12: note = X represents any amino acid residue
  VARIANT 13: note = X represents any amino acid residue
  VARIANT 14: note = X represents any amino acid residue
  VARIANT 15: note = X represents any amino acid residue
  VARIANT 16: note = X represents any amino acid residue
  VARIANT 18: note = X represents any amino acid residue
  VARIANT 19: note = X represents any amino acid residue
PXXPXXYXXXXXXXXXYXXL

SEQ ID NO: 27 - protein, 20 aa, synthetic construct
  REGION 1..20: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 8: note = X represents any amino acid residue
  VARIANT 9: note = X represents any amino acid residue
  VARIANT 10: note = X represents any amino acid residue
  VARIANT 11: note = X represents any amino acid residue
  VARIANT 12: note = X represents any amino acid residue
  VARIANT 13: note = X represents any amino acid residue
  VARIANT 14: note = X represents any amino acid residue
  VARIANT 15: note = X represents any amino acid residue
  VARIANT 18: note = X represents any amino acid residue
  VARIANT 19: note = X represents any amino acid residue
PXXPXXYXXXXXXXXDYXXL

SEQ ID NO: 28 - protein, 20 aa, synthetic construct
  REGION 1..20: note = Pilot peptide
  VARIANT 2: note = X represents any amino acid residue
  VARIANT 3: note = X represents any amino acid residue
  VARIANT 6: note = X represents any amino acid residue
  VARIANT 8: note = X represents any amino acid residue
  VARIANT 9: note = X represents any amino acid residue
  VARIANT 11: note = X represents any amino acid residue
  VARIANT 13: note = X represents any amino acid residue
  VARIANT 14: note = X represents any amino acid residue
  VARIANT 18: note = X represents any amino acid residue
  VARIANT 19: note = X represents any amino acid residue
PXXPEXYXXLXPXXPDYXXL

SEQ ID NO: 29 - protein, 9 aa, synthetic construct
  REGION 1..9: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any amino acid residue
  VARIANT 8: note = X represents any amino acid residue
PXXPXYXXL

SEQ ID NO: 30 - protein, 10 aa, synthetic construct
  REGION 1..10: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 8: note = X represents any amino acid residue
  VARIANT 9: note = X represents any amino acid residue
PXXPXDYXXL

SEQ ID NO: 31 - protein, 11 aa, synthetic construct
  REGION 1..11: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any one or several amino acid residue(s)
  VARIANT 9: note = X represents any amino acid residue
  VARIANT 10: note = X represents any amino acid residue
PXXPXYXYXXL

SEQ ID NO: 32 - protein, 12 aa, synthetic construct
  REGION 1..12: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any one or several amino acid residue(s)
  VARIANT 10: note = X represents any amino acid residue
  VARIANT 11: note = X represents any amino acid residue
PXXPXYXDYXXL

SEQ ID NO: 33 - protein, 20 aa, synthetic construct
  REGION 1..20: note = Pilot peptide
  VARIANT 2: note = X represents any amino acid residue
  VARIANT 3: note = X represents any amino acid residue
  VARIANT 6: note = X represents any one or several amino acid residue(s)
  VARIANT 8: note = X represents any amino acid residue
  VARIANT 9: note = X represents any amino acid residue
  VARIANT 11: note = X represents any one or several amino acid residue(s)
  VARIANT 13: note = X represents any amino acid residue
  VARIANT 14: note = X represents any amino acid residue
  VARIANT 18: note = X represents any amino acid residue
  VARIANT 19: note = X represents any amino acid residue
PXXPEXYXXLXPXXPDYXXL

SEQ ID NO: 34 - protein, 40 aa, synthetic construct
  REGION 1..40: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 7: note = X represents any amino acid residue
  VARIANT 8: note = X represents any amino acid residue
  VARIANT 10: note = X represents any amino acid residue
  VARIANT 11: note = X represents any amino acid residue
  VARIANT 13: note = X represents any amino acid residue
  VARIANT 14: note = X represents any amino acid residue
  VARIANT 15: note = X represents any amino acid residue
  VARIANT 17: note = X represents any amino acid residue
  VARIANT 18: note = X represents any amino acid residue
  VARIANT 20: note = X represents any amino acid residue
  VARIANT 22: note = X represents any amino acid residue
  VARIANT 23: note = X represents any amino acid residue
  VARIANT 26: note = X represents any amino acid residue
  VARIANT 28: note = X represents any amino acid residue
  VARIANT 29: note = X represents any amino acid residue
  VARIANT 31: note = X represents any amino acid residue
  VARIANT 33: note = X represents any amino acid residue
  VARIANT 34: note = X represents any amino acid residue
  VARIANT 38: note = X represents any amino acid residue
  VARIANT 39: note = X represents any amino acid residue
PXXPXXXXPXXPXXXYXXLXPXXPEXYXXLXPXXPDYXXL

SEQ ID NO: 35 - protein, 35 aa, synthetic construct
  REGION 1..35: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any amino acid residue
  VARIANT 8: note = X represents any amino acid residue
  VARIANT 10: note = X represents any one or several amino acid residue(s)
  VARIANT 12: note = X represents any amino acid residue
  VARIANT 13: note = X represents any amino acid residue
  VARIANT 15: note = X represents any one or several amino acid residue(s)
  VARIANT 17: note = X represents any amino acid residue
  VARIANT 18: note = X represents any amino acid residue
  VARIANT 21: note = X represents any one or several amino acid residue(s)
  VARIANT 23: note = X represents any amino acid residue
  VARIANT 24: note = X represents any amino acid residue
  VARIANT 26: note = X represents any one or several amino acid residue(s)
  VARIANT 28: note = X represents any amino acid residue
  VARIANT 29: note = X represents any amino acid residue
  VARIANT 33: note = X represents any amino acid residue
  VARIANT 34: note = X represents any amino acid residue
PXXPXPXXPXYXXLXPXXPEXYXXLXPXXPDYXXL

SEQ ID NO: 36 - protein, 44 aa, synthetic construct
  REGION 1..44: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 7: note = X represents any amino acid residue
  VARIANT 8: note = X represents any amino acid residue
  VARIANT 10: note = X represents any amino acid residue
  VARIANT 11: note = X represents any amino acid residue
  VARIANT 13: note = X represents any amino acid residue
  VARIANT 14: note = X represents any amino acid residue
  VARIANT 15: note = X represents any amino acid residue
  VARIANT 17: note = X represents any amino acid residue
  VARIANT 18: note = X represents any amino acid residue
  VARIANT 20: note = X represents any amino acid residue
  VARIANT 22: note = X represents any amino acid residue
  VARIANT 23: note = X represents any amino acid residue
  VARIANT 26: note = X represents any amino acid residue
  VARIANT 28: note = X represents any amino acid residue
  VARIANT 29: note = X represents any amino acid residue
  VARIANT 31: note = X represents any amino acid residue
  VARIANT 33: note = X represents any amino acid residue
  VARIANT 34: note = X represents any amino acid residue
  VARIANT 38: note = X represents any amino acid residue
  VARIANT 39: note = X represents any amino acid residue
  VARIANT 41: note = X represents any amino acid residue
  VARIANT 42: note = X represents any amino acid residue
  VARIANT 43: note = X represents any amino acid residue
  VARIANT 44: note = X represents any amino acid residue
PXXPXXXXPXXPXXXYXXLXPXXPEXYXXLXPXXPDYXXLXXXX

SEQ ID NO: 37 - protein, 39 aa, synthetic construct
  REGION 1..39: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any amino acid residue
  VARIANT 8: note = X represents any amino acid residue
  VARIANT 10: note = X represents any one or several mino acid residue(s)
  VARIANT 12: note = X represents any amino acid residue
  VARIANT 13: note = X represents any amino acid residue
  VARIANT 15: note = X represents any one or several amino acid residue(s)
  VARIANT 17: note = X represents any amino acid residue
  VARIANT 18: note = X represents any amino acid residue
  VARIANT 21: note = X represents any one or several amino acid residue(s)
  VARIANT 23: note = X represents any amino acid residue
  VARIANT 24: note = X represents any amino acid residue
  VARIANT 26: note = X represents any one or several amino acid residue(s)
  VARIANT 28: note = X represents any amino acid residue
  VARIANT 29: note = X represents any amino acid residue
  VARIANT 33: note = X represents any amino acid residue
  VARIANT 34: note = X represents any amino acid residue
  VARIANT 36: note = X represents any amino acid residue
  VARIANT 37: note = X represents any amino acid residue
  VARIANT 38: note = X represents any amino acid residue
  VARIANT 39: note = X represents any amino acid residue
PXXPXPXXPXYXXLXPXXPEXYXXLXPXXPDYXXLXXXX

SEQ ID NO: 38 - protein, 45 aa, synthetic construct
  REGION 1..45: note = Pilot peptide
APHFPEISFPPKPDSDYQALLPSAPEIYSHLSPTKPDYINLRPAP

SEQ ID NO: 39 - skipped

SEQ ID NO: 40 - protein, 159 aa, synthetic construct
  REGION 1..159: note = streptavidin
DPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 41 - protein, 38 aa, synthetic construct
  REGION 1..38: note = streptavidin-binding peptide
MDEKTTGWRGGHVVEGLAGELEQLRARLEHHPQGQREP

SEQ ID NO: 42 - protein, 29 aa, synthetic construct
  REGION 1..29: note = streptavidin-binding peptide
GGHVVEGLAGELEQLRARLEHHPQGQREP

SEQ ID NO: 43 - DNA, 789 bp, synthetic construct
  source 1..789: mol_type = other DNA
  misc_feature 1..789: note = Src streptavidin PP
atgggcagcagcaagagcaagcccaaggatcccagccagcggcggagaaaaagtagaggacctggcggtggatctggcggaggaagcggtggcggttcaggcggaggatctaccggtatggaccccagcaaggacagcaaggcccaagtgtctgctgccgaggctggaatcacaggcacctggtataatcagctgggcagcaccttcatcgtgaccgctggtgctgatggcgctctgacaggcacatatgagagcgccgtgggcaatgccgagagcagatatgtgctgaccggcagatacgatagcgcccctgccacagatggaagcggaacagctcttggatggaccgtggcctggaagaacaactacagaaacgcccacagcgccaccacttggagcggccaatatgttggcggagccgaggccagaatcaacacccaatggctgctgaccagcggcaccacagaggccaatgcctggaagtctacactcgtgggccacgacaccttcaccaaagtgaaacctagcgccgcctccatcgacgccgctaaaaaagccggcgtgaacaacggcaaccctctggatgctgttcagcaaggtggtggtagcggaggtggaagtggcggaggcagtggcggaggctctagaggcgcgccccacttccctgaaatctccttcccccctaaacccgattctgattatcaggccttgctaccatccgcgccagagatctactctcacctctcccccaccaaacccgattacatcaaccttcgaccggcgccctaa

SEQ ID NO: 44 - protein, 262 aa, synthetic construct
  REGION 1..262: note = Src streptavidin PP
MGSSKSKPKDPSQRRRKSRGPGGGSGGGSGGGSGGGSTGMDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQGGGSGGGSGGGSGGGSRGAPHFPEISFPPKPDSDYQALLPSAPEIYSHLSPTKPDYINLRPAP

SEQ ID NO: 45 - DNA, 822 bp, synthetic construct
  source 1..822: mol_type = other DNA
  misc_feature 1..822: note = Src Nanoluc PP
atgggcagcagcaagagcaagcccaaggatcccagccagcggcggagaaaaagtagaggacctggcggtggatctggcggaggaagcggtggcggttcaggcggaggatctaccggtatggtgttcaccctggaagatttcgtcggcgactggcggcagacagccggctataatctggaccaggtgctggaacaaggcggcgtgtccagcctgtttcagaacctgggagtgtccgtgacacccatccagagaatcgtgctgagcggcgagaacggcctgaagatcgacatccacgtgatcatcccttacgagggcctgtccggcgatcagatgggacagatcgagaagatctttaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacggcaacaagatcatcgacgagcggctgatcaaccccgatggcagcctgctgttcagagtgaccatcaacggcgtgacaggatggcggctgtgcgagagaattcttgccggtggtggtagcggaggtggaagtggcggaggcagtggcggaggctctagaggcgcgccccacttccctgaaatctccttcccccctaaacccgattctgattatcaggccttgctaccatccgcgccagagatctactctcacctctcccccaccaaacccgattacatcaaccttcgaccggcgccctaa

SEQ ID NO: 46 - protein, 273 aa, synthetic construct
  REGION 1..273: note = Src Nanoluc PP
MGSSKSKPKDPSQRRRKSRGPGGGSGGGSGGGSGGGSTGMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGGSGGGSGGGSGGGSRGAPHFPEISFPPKPDSDYQALLPSAPEIYSHLSPTKPDYINLRPAP

SEQ ID NO: 47 - DNA, 1353 bp, synthetic construct
  source 1..1353: mol_type = other DNA
  misc_feature 1..1353: note = Src asparaginase II PP
atgggcagcagcaagagcaagcccaaggatcccagccagcggcggagaaaaagtagaggacctggcggtggatctggcggaggaagcggtggcggttcaggcggaggatctaccggtatggaattcttcaagaaaacagccctggccgctctggtcatgggcttttctggtgctgctctggccctgcctaacatcaccattctggctaccggcggcacaatagctggcggcggagattctgccaccaagagcaattacaccgtgggcaaagtgggcgtcgagaacctggttaatgccgtgcctcagctgaaggacattgccaacgtgaagggcgagcaggtcgtgaacatcggcagccaggacatgaacgacaacgtgtggctgaccctggctaagaagatcaacaccgactgcgacaagaccgacggcttcgtgatcacccacggcaccgacaccatggaagagacagcctacttcctggacctgaccgtgaagtgcgacaagcccgtggttatggtcggagccatgaggcctagcaccagcatgtctgccgacggacccttcaacctgtacaacgccgtggttacagccgccgataaggcctctgctaatagaggcgtgctggtcgtgatgaacgataccgtgctggacggcagggacgtgaccaagaccaataccaccgacgtggcaaccttcaagagcgtgaactatggccctctgggctacatccacaacggcaagatcgactaccagcggacccctgccagaaagcacaccagcgatacccctttcgacgtgtccaagctgaacgagctgcctaaagtgggcatcgtgtacaactacgccaacgccagcgacctgcctgccaaagctcttgtggatgccggctacgacggaatcgtgtcagccggcgttggcaacggcaatctgtacaagtccgtgttcgacaccctggcaaccgccgccaaaacaggcacagccgtcgtcagatctagcagagtgcctacaggcgccaccacacaggatgccgaagtggacgatgccaaatacggctttgtggcctccggcacactgaaccctcagaaagccagagtgctgctccagctggccctgacacagaccaaggatccccagcagattcagcagatcttcaaccagtacggtggtggtagcggaggtggaagtggcggaggcagtggcggaggctctagaggcgcgccccacttccctgaaatctccttcccccctaaacccgattctgattatcaggccttgctaccatccgcgccagagatctactctcacctctcccccaccaaacccgattacatcaaccttcgaccggcgccctaa

SEQ ID NO: 48 - protein, 450 aa, synthetic construct
  REGION 1..450: note = Src asparaginase II PP
MGSSKSKPKDPSQRRRKSRGPGGGSGGGSGGGSGGGSTGMEFFKKTALAALVMGFSGAALALPNITILATGGTIAGGGDSATKSNYTVGKVGVENLVNAVPQLKDIANVKGEQVVNIGSQDMNDNVWLTLAKKINTDCDKTDGFVITHGTDTMEETAYFLDLTVKCDKPVVMVGAMRPSTSMSADGPFNLYNAVVTAADKASANRGVLVVMNDTVLDGRDVTKTNTTDVATFKSVNYGPLGYIHNGKIDYQRTPARKHTSDTPFDVSKLNELPKVGIVYNYANASDLPAKALVDAGYDGIVSAGVGNGNLYKSVFDTLATAAKTGTAVVRSSRVPTGATTQDAEVDDAKYGFVASGTLNPQKARVLLQLALTQTKDPQQIQQIFNQYGGGSGGGSGGGSGGGSRGAPHFPEISFPPKPDSDYQALLPSAPEIYSHLSPTKPDYINLRPAP

SEQ ID NO: 49 - DNA, 1137 bp, synthetic construct
  source 1..1137: mol_type = other DNA
  misc_feature 1..1137: note = ZeoR-PT2A-SBP-Nanoluc
atggccaagcttacatctgctgtgcctgtgctgaccgccagagatgttgctggcgccgtggaattctggaccgacagactgggcttcagccgggacttcgtggaagatgattttgccggcgtcgtgcgggacgacgtgaccctgtttattagcgccgtgcaggaccaggtggtgcccgataatactctggcctgggtctgggttcgaggcctggatgaactgtatgccgagtggagcgaggtggtgtccaccaacttcagagatgccagcggacctgccatgaccgagattggagaacagccttggggcagagagttcgccctgagagatcctgccggaaactgcgtgcacttcgtggccgaagaacaggatggcggaggttctggcggaggaagcggtggcggatcaggcggaggatctgccacaaatttcagcctgctgaagcaggccggcgacgtggaagaaaatcctggacctggcggacacgtggtggaaggacttgctggcgaactggaacagctgcgggccagactggaacaccatcctcagggacaaagagagcctggcggcggtagcggcggtggcagtggtggtggtagtggcggcggatctacctgcaggatggtctttaccctggaagatttcgtcggcgactggcggcagacagccggctataatctggatcaggtgctggaacaaggcggcgtgtccagcctgtttcagaacctgggagtgtccgtgacacccatccagagaatcgtgctgagcggcgagaacggcctgaagatcgacatccacgtgatcatcccttacgagggcctgtccggcgatcagatgggacagatcgagaagatctttaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacggcaacaagatcatcgacgagcggctgatcaaccccgatggctccctgctgttcagagtgaccatcaacggcgttacaggctggcggctgtgcgagagaattctggcttaa

SEQ ID NO: 50 - protein, 378 aa, synthetic construct
  REGION 1..378: note = ZeoR-PT2A-SBP-Nanoluc
MAKLTSAVPVLTARDVAGAVEFWTDRLGFSRDFVEDDFAGVVRDDVTLFISAVQDQVVPDNTLAWVWVRGLDELYAEWSEVVSTNFRDASGPAMTEIGEQPWGREFALRDPAGNCVHFVAEEQDGGGSGGGSGGGSGGGSATNFSLLKQAGDVEENPGPGGHVVEGLAGELEQLRARLEHHPQGQREPGGGSGGGSGGGSGGGSTCRMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 51 - DNA, 3555 bp, synthetic construct
  source 1..3555: mol_type = other DNA
  misc_feature 1..3555: note = ZeoR-PT2A-SBP-eIF4G
atggccaagcttacatctgctgtgcctgtgctgaccgccagagatgttgctggcgccgtggaattctggaccgacagactgggcttcagccgggacttcgtggaagatgattttgccggcgtcgtgcgggacgacgtgaccctgtttattagcgccgtgcaggaccaggtggtgcccgataatactctggcctgggtctgggttcgaggcctggatgaactgtatgccgagtggagcgaggtggtgtccaccaacttcagagatgccagcggacctgccatgaccgagattggagaacagccttggggcagagagttcgccctgagagatcctgccggaaactgcgtgcacttcgtggccgaagaacaggatggcggaggttctggcggaggaagcggtggcggatcaggcggaggatctgccacaaatttcagcctgctgaagcaggccggcgacgtggaagaaaatcctggacctggcggacacgtggtggaaggacttgctggcgaactggaacagctgcgggccagactggaacaccatcctcagggacaaagagagcctggcggcggtagcggcggtggcagtggtggtggtagtggcggcggatctacctgcaggttcgccagcatgcagaagcctgaaggcctgcctcacatcagcgacgtggtgctggataaggccaacaagacccctctgaggcctctggaccctacaagactgcagggcatcaactgcggccctgacttcacacccagcttcgccaatctgggcagaaccacactgagcacaagaggccctccaagaggtggacctggcggagaacttcctagaggacctcaggctggactgggccctagaagatctcagcagggccccagaaaagagccccggaagatcattgccaccgtgctgatgaccgaggacatcaagctgaacaaggccgagaaggcctggaagcccagcagcaaaagaacagccgccgacaaggacagaggcgaagaggatgccgatggcagcaagacccaggacctgttcagaagagtgcggagcatcctgaacaagctgacccctcagatgttccagcagctgatgaagcaagtgacccagctggctatcgacaccgaggaaagactgaagggcgtcatcgacctgatctttgagaaggccatcagcgagcccaacttcagcgtggcctacgccaacatgtgccggtgtctgatggccctgaaggtgccaaccaccgagaagcctaccgtgaccgtgaacttcagaaagctgctgctgaatcggtgccagaaagagttcgagaaggacaaggacgacgacgaggtgttcgagaaaaagcagaaagagatggacgaggccgccaccgccgaagaaagaggcagactgaaagaggaactggaagaagccagagatatcgccagaaggcggagcctgggcaacatcaagtttatcggcgagctgtttaagctgaagatgctgacagaggccatcatgcacgactgcgtggtcaagctgctgaagaaccacgacgaggaatccctggaatgcctgtgcagactgctgaccaccatcggcaaggacctggacttcgagaaagccaagcctcggatggaccagtacttcaaccagatggaaaagatcatcaaagagaagaaaaccagcagccgcatccggttcatgctgcaggatgtgctggatctgagaggcagcaactgggtgcccagaagaggcgatcagggccctaagaccatcgaccagatccacaaagaggccgagatggaagaacaccgcgagcacatcaaggtgcagcagctcatggctaagggcagcgacaagcgtagaggcggacctcctggacctccaatcagtagaggactgcccctggtggatgacggcggatggaataccgtgcctatcagcaagggcagcagaccaatcgacaccagcagactgaccaagatcaccaagcctggcagcatcgacagcaacaaccagctgtttgctcctggcggcagactgtcttggggcaagggatcttctggtggctctggcgccaaaccttctgatgccgcttctgaagctgcccggcctgccacaagcaccctgaatagattttcagccctgcagcaggccgtgcctaccgagagcaccgacaatagaagagtggtgcagagaagcagcctgagcagagagagaggcgaaaaggctggcgacaggggcgacagactggaaagaagtgaaagaggcggcgatagaggcgaccggctggatagagctagaacccctgccaccaagcggagcttcagcaaagaggtcgaggaacggtccagagagcggcctagtcaacctgagggactgagaaaagccgccagcctgactgaggacagagacagaggtagagatgccgtgaagcgggaagctgctctgcctcctgtgtctcctctgaaagccgctctgagcgaggaagaactggaaaagaaatccaaggccattatcgaggaatacctgcacctgaacgacatgaaggaagccgtgcagtgcgtgcaagagctggcctcacctagcctgctgttcatctttgtgcggcacggcgtggaatctaccctggaaagatctgccattgccagagaacacatgggccagctgctccaccaactgctgtgtgccggacatctgagcacagcccagtactaccagggcctgtacgagatcctggaactggccgaggatatggaaatcgacatccctcacgtgtggctgtacctggccgagctggtcacaccaattctgcaagagggcggagtgcctatgggagagctgttcagagagatcacaaagcccctgcggcctctgggcaaagctgcatctctgctgctcgagattctgggcctgctgtgcaagtctatgggccccaagaaagtgggcaccctttggagagaagccggcctgtcttggaaagagtttctgcccgaaggccaggacatcggcgcctttgtggccgagcagaaggtcgagtataccctgggcgaagagtctgaggctccaggccaaagagcactgcctagcgaggaactgaaccggcagctggaaaaactgctgaaagaaggcagcagcaaccagagagtgttcgactggatcgaggccaacctgagcgaacagcagatcgtgtctaacacccttgtgcgcgccctgatgacagccgtgtgttacagcgccatcatcttcgagacacccctgagagtggatgtggccgtgctgaaggccagagctaaactgctgcagaaatacctgtgcgacgaacagaaagagctgcaggccctgtacgctctgcaggctctggtggttacactggaacagcctccaaacctgctgaggatgttcttcgacgccctgtatgacgaggacgtggtcaaagaggacgccttctacagctgggagagcagcaaggatcctgccgaacagcaaggcaaaggcgtggcactgaagtccgtgaccgccttcttcaagtggctgcgggaagccgaggaagagagcgaccataactga

SEQ ID NO: 52 - protein, 1184 aa, synthetic construct
  REGION 1..1184: note = ZeoR-PT2A-SBP-eIF4G
MAKLTSAVPVLTARDVAGAVEFWTDRLGFSRDFVEDDFAGVVRDDVTLFISAVQDQVVPDNTLAWVWVRGLDELYAEWSEVVSTNFRDASGPAMTEIGEQPWGREFALRDPAGNCVHFVAEEQDGGGSGGGSGGGSGGGSATNFSLLKQAGDVEENPGPGGHVVEGLAGELEQLRARLEHHPQGQREPGGGSGGGSGGGSGGGSTCRFASMQKPEGLPHISDVVLDKANKTPLRPLDPTRLQGINCGPDFTPSFANLGRTTLSTRGPPRGGPGGELPRGPQAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 53 - DNA, 1713 bp, synthetic construct
  source 1..1713: mol_type = other DNA
  misc_feature 1..1713: note = ZeoR-PT2A-SBP-Oct4
atggccaagcttacatctgctgtgcctgtgctgaccgccagagatgttgctggcgccgtggaattctggaccgacagactgggcttcagccgggacttcgtggaagatgattttgccggcgtcgtgcgggacgacgtgaccctgtttattagcgccgtgcaggaccaggtggtgcccgataatactctggcctgggtctgggttcgaggcctggatgaactgtatgccgagtggagcgaggtggtgtccaccaacttcagagatgccagcggacctgccatgaccgagattggagaacagccttggggcagagagttcgccctgagagatcctgccggaaactgcgtgcacttcgtggccgaagaacaggatggcggaggttctggcggaggaagcggtggcggatcaggcggaggatctgccacaaatttcagcctgctgaagcaggccggcgacgtggaagaaaatcctggacctggcggacacgtggtggaaggacttgctggcgaactggaacagctgcgggccagactggaacaccatcctcagggacaaagagagcctggcggcggtagcggcggtggcagtggtggtggtagtggcggcggatctacctgcaggttcgccagcatggctggacatctggcctccgacttcgccttctctccaccacctggcggaggcggagatggaccaggtggacctgaacctggatgggttgaccctagaacctggctgagctttcagggacctcctggcggacctggaattggacctggtgttggccctggctctgaagtgtggggaatccctccttgtcctccaccttacgagttctgtggcggcatggcctactgtggccctcaagttggagttggcctggtgcctcaaggcggcctggaaacatctcagcctgagggcgaagctggcgtgggcgtcgagtctaattctgatggcgcctctcctgagccttgcaccgttacacctggcgccgtgaagctggaaaaagagaaactggaacagaaccccgaggaaagccaggacatcaaggccctgcagaaagagctggaacagttcgccaagctgctgaagcagaagcggatcaccctgggctacacacaggctgatgtgggcctgacactgggcgtgctgtttggcaaggtgttcagccagaccaccatctgtagattcgaagccctgcagctgagcttcaagaacatgtgcaagctgcggcccctgctgcagaaatgggttgaagaggccgacaacaacgagaacctgcaagagatctgcaaggccgagacactggtgcaggcccggaagagaaagagaaccagcatcgagaacagagtgcggggcaacctggaaaacctgttcctgcagtgccccaagcctacactgcagcagatcagccacattgcccagcagctgggactcgaaaaggacgtcgtcagagtgtggttctgcaaccggcggcagaagggcaagagaagcagcagcgattacgcccagagagaggactttgaggccgctggcagtcctttttctggcggccctgtgtcctttcctctggctcctggacctcactttggcacacctggctatggcagccctcacttcacagccctgtacagcagcgtgccctttccagaaggcgaggcctttcctcctgtgtccgtgacaacactgggcagccctatgcacagcaactga

SEQ ID NO: 54 - protein, 570 aa, synthetic construct
  REGION 1..570: note = ZeoR-PT2A-SBP-Oct4
MAKLTSAVPVLTARDVAGAVEFWTDRLGFSRDFVEDDFAGVVRDDVTLFISAVQDQVVPDNTLAWVWVRGLDELYAEWSEVVSTNFRDASGPAMTEIGEQPWGREFALRDPAGNCVHFVAEEQDGGGSGGGSGGGSGGGSATNFSLLKQAGDVEENPGPGGHVVEGLAGELEQLRARLEHHPQGQREPGGGSGGGSGGGSGGGSTCRFASMAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 55 - DNA, 1221 bp, synthetic construct
  source 1..1221: mol_type = other DNA
  misc_feature 1..1221: note = SBP Oct4
atgggcggacacgtggtggaaggacttgctggcgaactggaacagctgcgggccagactggaacaccatcctcagggacaaagagagcctggcggcggtagcggcggtggcagtggtggtggtagtggcggcggatctatggctggacatctggcctccgacttcgccttctctccaccacctggcggaggcggagatggaccaggtggacctgaacctggatgggttgaccctagaacctggctgagctttcagggacctcctggcggacctggaattggacctggtgttggccctggctctgaagtgtggggaatccctccttgtcctccaccttacgagttctgtggcggcatggcctactgtggccctcaagttggagttggcctggtgcctcaaggcggcctggaaacatctcagcctgagggcgaagctggcgtgggcgtcgagtctaattctgatggcgcctctcctgagccttgcaccgttacacctggcgccgtgaagctggaaaaagagaaactggaacagaaccccgaggaaagccaggacatcaaggccctgcagaaagagctggaacagttcgccaagctgctgaagcagaagcggatcaccctgggctacacacaggctgatgtgggcctgacactgggcgtgctgtttggcaaggtgttcagccagaccaccatctgtagattcgaagccctgcagctgagcttcaagaacatgtgcaagctgcggcccctgctgcagaaatgggttgaagaggccgacaacaacgagaacctgcaagagatctgcaaggccgagacactggtgcaggcccggaagagaaagagaaccagcatcgagaacagagtgcggggcaacctggaaaacctgttcctgcagtgccccaagcctacactgcagcagatcagccacattgcccagcagctgggactcgaaaaggacgtcgtcagagtgtggttctgcaaccggcggcagaagggcaagagaagcagcagcgattacgcccagagagaggactttgaggccgctggcagtcctttttctggcggccctgtgtcctttcctctggctcctggacctcactttggcacacctggctatggcagccctcacttcacagccctgtacagcagcgtgccctttccagaaggcgaggcctttcctcctgtgtccgtgacaacactgggcagccctatgcacagcaactga

SEQ ID NO: 56 - protein, 406 aa, synthetic construct
  REGION 1..406: note = SBP Oct4
MGGHVVEGLAGELEQLRARLEHHPQGQREPGGGSGGGSGGGSGGGSMAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 57 - DNA, 1497 bp, synthetic construct
  source 1..1497: mol_type = other DNA
  misc_feature 1..1497: note = OMOMYC-SBP
atggacttcttccgcgtggtggaaaaccagcagcctcctgccacaatgcccctgaacgtgtccttcaccaaccggaactacgacctggactacgacagcgtgcagccctacttctactgcgacgaggaagagaacttctaccagcagcagcaacagagcgaactccagcctccagctcctagcgaggacatctggaagaagttcgagctgctgcccacacctcctctgagccctagtagaagatccggcctgtgcagccccagctatgtggccgtgacaccttttagcctgcggggcgataatgatggcggcggaggcagctttagcaccgccgatcaactggaaatggtcacagagctgctcggcggcgacatggtcaaccagagcttcatctgcgaccccgacgacgagacattcatcaagaacatcatcatccaggactgcatgtggagcggctttagcgccgctgccaagctggtgtctgagaagctggcctcttatcaggccgccagaaaggatagcggcagccccaatcctgccagaggccactctgtgtgtagcacctccagcctgtacctgcaagatctgtctgccgccgcttccgagtgcatcgatcctagcgtggtgttcccctatcctctgaacgacagcagctcccctaagagctgtgccagccaggatagcagcgctttcagccctagcagcgatagcctgctgagcagcacagagtctagccctcagggctctcctgaacctctggtgctgcacgaggaaacccctccaaccaccagcagcgacagcgaggaagaacaagaggacgaagaggaaatcgacgtcgtcagcgtggaaaagagacaggcccctggcaagagaagcgagtctggctctccttctgccggcggacactctaagcctccacattctccactggtgctgaagcggtgccacgtgtccacacaccagcacaattatgccgctcctccaagcacacggaaggactatcctgccgccaagagagtgaagctggatagcgtcagagtgctgcggcagatcagcaacaaccggaagtgcacaagccccagaagctccgacaccgaggaaaacgtgaagcggagaacccacaacgtgctggaacggcagagaagaaacgagctgaagcgcagcttcttcgccctgagagatcagatccccgagctggaaaacaacgagaaggcccctaaggtggtcatcctgaagaaggccaccgcctacatcctgagcgtgcaggccgaaacacagaagctgatctccgagatcgacctgctgcggaagcagaacgagcagctgaagcacaagctggaacagctgagaaacagctgcgccggcggtggatctggcggaggaagcggtggcggttcaggcggaggatctggcggacacgtggtggaaggacttgctggcgaactggaacagctgcgggccagactggaacaccatcctcagggacaaagagagcct

SEQ ID NO: 58 - protein, 499 aa, synthetic construct
  REGION 1..499: note = OMOMYC-SBP
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAETQKLISEIDLLRKQNEQLKHKLEQLRNSCAGGGSGGGSGGGSGGGSGGHVVEGLAGELEQLRARLEHHPQGQREP

SEQ ID NO: 59 - DNA, 1179 bp, synthetic construct
  source 1..1179: mol_type = other DNA
  misc_feature 1..1179: note = Asparaginase II-SBP
atggaattcttcaagaaaacagccctggccgctctggtcatgggcttttctggtgctgctctggccctgcctaacatcaccattctggctaccggcggcacaatagctggcggcggagattctgccaccaagagcaattacaccgtgggcaaagtgggcgtcgagaacctggttaatgccgtgcctcagctgaaggacattgccaacgtgaagggcgagcaggtcgtgaacatcggcagccaggacatgaacgacaacgtgtggctgaccctggctaagaagatcaacaccgactgcgacaagaccgacggcttcgtgatcacccacggcaccgacaccatggaagagacagcctacttcctggacctgaccgtgaagtgcgacaagcccgtggttatggtcggagccatgaggcctagcaccagcatgtctgccgacggacccttcaacctgtacaacgccgtggttacagccgccgataaggcctctgctaatagaggcgtgctggtcgtgatgaacgataccgtgctggacggcagggacgtgaccaagaccaataccaccgacgtggcaaccttcaagagcgtgaactatggccctctgggctacatccacaacggcaagatcgactaccagcggacccctgccagaaagcacaccagcgatacccctttcgacgtgtccaagctgaacgagctgcctaaagtgggcatcgtgtacaactacgccaacgccagcgacctgcctgccaaagctcttgtggatgccggctacgacggaatcgtgtcagccggcgttggcaacggcaatctgtacaagtccgtgttcgacaccctggcaaccgccgccaaaacaggcacagccgtcgtcagatctagcagagtgcctacaggcgccaccacacaggatgccgaagtggacgatgccaaatacggctttgtggcctccggcacactgaaccctcagaaagccagagtgctgctccagctggccctgacacagaccaaggatccccagcagattcagcagatcttcaaccagtacggcggtggatctggcggaggaagcggtggcggttcaggcggaggatctggcggacacgtggtggaaggacttgctggcgaactggaacagctgcgggccagactggaacaccatcctcagggacaaagagagcct

SEQ ID NO: 60 - protein, 393 aa, synthetic construct
  REGION 1..393: note = Asparaginase II-SBP
MEFFKKTALAALVMGFSGAALALPNITILATGGTIAGGGDSATKSNYTVGKVGVENLVNAVPQLKDIANVKGEQVVNIGSQDMNDNVWLTLAKKINTDCDKTDGFVITHGTDTMEETAYFLDLTVKCDKPVVMVGAMRPSTSMSADGPFNLYNAVVTAADKASANRGVLVVMNDTVLDGRDVTKTNTTDVATFKSVNYGPLGYIHNGKIDYQRTPARKHTSDTPFDVSKLNELPKVGIVYNYANASDLPAKALVDAGYDGIVSAGVGNGNLYKSVFDTLATAAKTGTAVVRSSRVPTGATTQDAEVDDAKYGFVASGTLNPQKARVLLQLALTQTKDPQQIQQIFNQYGGGSGGGSGGGSGGGSGGHVVEGLAGELEQLRARLEHHPQGQREP

SEQ ID NO: 61 - DNA, 1980 bp, synthetic construct
  source 1..1980: mol_type = other DNA
  misc_feature 1..1980: note = Oct4-SBP-eGFP
atggctggacatctggcctccgacttcgccttctctccaccacctggcggaggcggagatggaccaggtggacctgaacctggatgggttgaccctagaacctggctgagctttcagggacctcctggcggacctggaattggacctggtgttggccctggctctgaagtgtggggaatccctccttgtcctccaccttacgagttctgtggcggcatggcctactgtggccctcaagttggagttggcctggtgcctcaaggcggcctggaaacatctcagcctgagggcgaagctggcgtgggcgtcgagtctaattctgatggcgcctctcctgagccttgcaccgttacacctggcgccgtgaagctggaaaaagagaaactggaacagaaccccgaggaaagccaggacatcaaggccctccagaaagagctggaacagttcgccaagctgctgaagcagaagcggatcaccctgggctacacacaggctgatgtgggcctgacactgggcgtgctgtttggcaaggtgttcagccagaccacaatctgtagattcgaagccctccagctgagcttcaagaacatgtgcaagctgcggcccctgctccagaaatgggttgaagaggccgacaacaacgagaacctgcaagagatctgcaaggccgagacactggtgcaggcccggaagagaaagagaaccagcatcgagaacagagtgcggggcaacctggaaaacctgttcctgcaatgccccaagcctacactccagcagatcagccacattgcccagcagctgggactcgaaaaggacgtcgtcagagtgtggttctgcaaccggcggcagaagggcaagagaagcagcagcgattacgcccagagagaggactttgaggccgctggcagtcctttttctggcggccctgtgtcctttcctctggctcctggacctcactttggcacacctggctatggcagccctcacttcacagccctgtacagcagcgtgccctttccagaaggcgaggcctttcctcctgtgtccgtgacaacactgggcagccccatgcacagcaatggcggcggtagcggcggtggcagtggtggtggtagtggcggcggatctggcggccatgtggttgaaggacttgccggcgaactggaacagctgagagcccggcttgagcaccatcctcagggacaaagagaacctggcggaggaagcggtggcggatcaggtggtggatctggcggcggatctatggtgtccaagggcgaagaactgttcaccggcgtggtgcccattctggtggaactggatggggatgtgaacggccacaagttcagcgttagcggagaaggcgaaggcgacgccacatacggaaagctgaccctgaagttcatctgcaccaccggcaagctgcctgtgccttggcctacactggtcacaaccctgacatacggcgtgcagtgcttcagcagataccccgaccatatgaagcagcacgacttcttcaagagcgccatgcctgagggctacgtgcaagagcggaccatcttctttaaggacgacggcaactacaagaccagggccgaagtgaagttcgagggcgacaccctggtcaaccggatcgagctgaagggcatcgacttcaaagaggacggcaacatcctgggccacaagcttgagtacaactacaacagccacaacgtgtacatcatggccgacaagcagaaaaacggcatcaaagtgaacttcaagatccggcacaacatcgaggacggctctgtgcagctggccgatcactaccagcagaacacacccatcggagatggccctgtgctgctgcccgataaccactacctgagcacacagagcgccctgagcaaggaccccaacgagaagagggatcacatggtgctgctggaattcgtgaccgccgctggcatcacactcggcatggatgagctgtacaag

SEQ ID NO: 62 - protein, 660 aa, synthetic construct
  REGION 1..660: note = Oct4-SBP-eGFP
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSNGGGSGGGSGGGSGGGSGGHVVEGLAGELEQLRARLEHHPQGQREPGGGSGGGSGGGSGGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 63 - DNA, 1983 bp, synthetic construct
  source 1..1983: mol_type = other DNA
  misc_feature 1..1983: note = SBP-Oct4-eGFP
atgggcggccatgtggttgaaggacttgccggcgaactggaacagctgagagcccggcttgagcaccatcctcagggacaaagagaacctggcggaggaagcggtggcggatcaggtggtggatctggcggcggatctatggctggacatctggcctccgacttcgccttctctccaccacctggcggaggcggagatggaccaggtggacctgaacctggatgggttgaccctagaacctggctgagctttcagggacctcctggcggacctggaattggacctggtgttggccctggctctgaagtgtggggaatccctccttgtcctccaccttacgagttctgtggcggcatggcctactgtggccctcaagttggagttggcctggtgcctcaaggcggcctggaaacatctcagcctgagggcgaagctggcgtgggcgtcgagtctaattctgatggcgcctctcctgagccttgcaccgttacacctggcgccgtgaagctggaaaaagagaaactggaacagaaccccgaggaaagccaggacatcaaggccctccagaaagagctggaacagttcgccaagctgctgaagcagaagcggatcaccctgggctacacacaggctgatgtgggcctgacactgggcgtgctgtttggcaaggtgttcagccagaccaccatctgtagattcgaagccctccagctgagcttcaagaacatgtgcaagctgcggcccctgctccagaaatgggttgaagaggccgacaacaacgagaacctgcaagagatctgcaaggccgagacactggtgcaggcccggaagagaaagagaaccagcatcgagaacagagtgcggggcaacctggaaaacctgttcctgcaatgccccaagcctacactccagcagatcagccacattgcccagcagctgggactcgaaaaggacgtcgtcagagtgtggttctgcaaccggcggcagaagggcaagagaagcagcagcgattacgcccagagagaggactttgaggccgctggcagtcctttttctggcggccctgtgtcctttcctctggctcctggacctcactttggcacacctggctatggcagccctcacttcacagccctgtacagcagcgtgccctttccagaaggcgaggcctttcctcctgtgtccgtgacaacactgggcagccccatgcacagcaatggcggcggtagcggcggtggcagtggtggtggtagtggcggcggatctatggtgtccaagggcgaagaactgttcaccggcgtggtgcccattctggtggaactggatggggatgtgaacggccacaagttcagcgttagcggagaaggcgaaggcgacgccacatacggaaagctgaccctgaagttcatctgcaccaccggcaagctgcctgtgccttggcctacactggtcacaaccctgacatacggcgtgcagtgcttcagcagataccccgaccatatgaagcagcacgacttcttcaagagcgccatgcctgagggctacgtgcaagagcggaccatcttctttaaggacgacggcaactacaagaccagggccgaagtgaagttcgagggcgacaccctggtcaaccggatcgagctgaagggcatcgacttcaaagaggacggcaacatcctgggccacaagcttgagtacaactacaacagccacaacgtgtacatcatggccgacaagcagaaaaacggcatcaaagtgaacttcaagatccggcacaacatcgaggacggctctgtgcagctggccgatcactaccagcagaacacacccatcggagatggccctgtgctgctgcccgataaccactacctgagcacacagagcgccctgagcaaggaccccaacgagaagagggatcacatggtgctgctggaattcgtgaccgccgctggcatcacactcggcatggatgagctgtacaag

SEQ ID NO: 64 - protein, 661 aa, synthetic construct
  REGION 1..661: note = SBP-Oct4-eGFP
MGGHVVEGLAGELEQLRARLEHHPQGQREPGGGSGGGSGGGSGGGSMAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSNGGGSGGGSGGGSGGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 65 - DNA, 855 bp, synthetic construct
  source 1..855: mol_type = other DNA
  misc_feature 1..855: note = SBP-eGFP
atgggcggccatgtggttgaaggacttgccggcgaactggaacagctgagagcccggcttgagcaccatcctcagggacaaagagaacctggcggaggaagcggtggcggatcaggtggtggatctggcggcggatctatggtgtccaagggcgaagaactgttcaccggcgtggtgcccattctggtggaactggatggggatgtgaacggccacaagttcagcgttagcggagaaggcgaaggcgacgccacatacggaaagctgaccctgaagttcatctgcaccaccggcaagctgcctgtgccttggcctacactggtcacaaccctgacatacggcgtgcagtgcttcagcagataccccgaccatatgaagcagcacgacttcttcaagagcgccatgcctgagggctacgtgcaagagcggaccatcttctttaaggacgacggcaactacaagaccagggccgaagtgaagttcgagggcgacaccctggtcaaccggatcgagctgaagggcatcgacttcaaagaggacggcaacatcctgggccacaagcttgagtacaactacaacagccacaacgtgtacatcatggccgacaagcagaaaaacggcatcaaagtgaacttcaagatccggcacaacatcgaggacggctctgtgcagctggccgatcactaccagcagaacacacccatcggagatggccctgtgctgctgcccgataaccactacctgagcacacagagcgccctgagcaaggaccccaacgagaagagggatcacatggtgctgctggaattcgtgaccgccgctggcatcacactcggcatggatgagctgtacaag

SEQ ID NO: 66 - protein, 285 aa, synthetic construct
  REGION 1..285: note = SBP-eGFP
MGGHVVEGLAGELEQLRARLEHHPQGQREPGGGSGGGSGGGSGGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 67 - protein, 360 aa, synthetic construct
  REGION 1..360: note = Oct4
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 68 - DNA, 1083 bp, synthetic construct
  source 1..1083: mol_type = other DNA
  misc_feature 1..1083: note = Oct4
atggctggacatctggcctccgacttcgccttctctccaccacctggcggaggcggagatggaccaggtggacctgaacctggatgggttgaccctagaacctggctgagctttcagggacctcctggcggacctggaattggacctggtgttggccctggctctgaagtgtggggaatccctccttgtcctccaccttacgagttctgtggcggcatggcctactgtggccctcaagttggagttggcctggtgcctcaaggcggcctggaaacatctcagcctgagggcgaagctggcgtgggcgtcgagtctaattctgatggcgcctctcctgagccttgcaccgttacacctggcgccgtgaagctggaaaaagagaaactggaacagaaccccgaggaaagccaggacatcaaggccctgcagaaagagctggaacagttcgccaagctgctgaagcagaagcggatcaccctgggctacacacaggctgatgtgggcctgacactgggcgtgctgtttggcaaggtgttcagccagaccaccatctgtagattcgaagccctgcagctgagcttcaagaacatgtgcaagctgcggcccctgctgcagaaatgggttgaagaggccgacaacaacgagaacctgcaagagatctgcaaggccgagacactggtgcaggcccggaagagaaagagaaccagcatcgagaacagagtgcggggcaacctggaaaacctgttcctgcagtgccccaagcctacactgcagcagatcagccacattgcccagcagctgggactcgaaaaggacgtcgtcagagtgtggttctgcaaccggcggcagaagggcaagagaagcagcagcgattacgcccagagagaggactttgaggccgctggcagtcctttttctggcggccctgtgtcctttcctctggctcctggacctcactttggcacacctggctatggcagccctcacttcacagccctgtacagcagcgtgccctttccagaaggcgaggcctttcctcctgtgtccgtgacaacactgggcagccctatgcacagcaactga

SEQ ID NO: 69 - protein, 239 aa, synthetic construct
  REGION 1..239: note = eEGFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 70 - DNA, 717 bp, synthetic construct
  source 1..717: mol_type = other DNA
  misc_feature 1..717: note = eEGFP
atggtgtccaagggcgaagaactgttcaccggcgtggtgcccattctggtggaactggatggggatgtgaacggccacaagttcagcgttagcggagaaggcgaaggcgacgccacatacggaaagctgaccctgaagttcatctgcaccaccggcaagctgcctgtgccttggcctacactggtcacaaccctgacatacggcgtgcagtgcttcagcagataccccgaccatatgaagcagcacgacttcttcaagagcgccatgcctgagggctacgtgcaagagcggaccatcttctttaaggacgacggcaactacaagaccagggccgaagtgaagttcgagggcgacaccctggtcaaccggatcgagctgaagggcatcgacttcaaagaggacggcaacatcctgggccacaagcttgagtacaactacaacagccacaacgtgtacatcatggccgacaagcagaaaaacggcatcaaagtgaacttcaagatccggcacaacatcgaggacggctctgtgcagctggccgatcactaccagcagaacacacccatcggagatggccctgtgctgctgcccgataaccactacctgagcacacagagcgccctgagcaaggaccccaacgagaagagggatcacatggtgctgctggaattcgtgaccgccgctggcatcacactcggcatggatgagctgtacaag

SEQ ID NO: 71 - protein, 171 aa, synthetic construct
  REGION 1..171: note = Nanoluc
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 72 - DNA, 513 bp, synthetic construct
  source 1..513: mol_type = other DNA
  misc_feature 1..513: note = Nanoluc
atggtgttcaccctggaagatttcgtcggcgactggcggcagacagccggctataatctggaccaggtgctggaacaaggcggcgtgtccagcctgtttcagaacctgggagtgtccgtgacacccatccagagaatcgtgctgagcggcgagaacggcctgaagatcgacatccacgtgatcatcccttacgagggcctgtccggcgatcagatgggacagatcgagaagatctttaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacggcaacaagatcatcgacgagcggctgatcaaccccgatggcagcctgctgttcagagtgaccatcaacggcgtgacaggatggcggctgtgcgagagaattcttgcc

SEQ ID NO: 73 - protein, 317 aa, synthetic construct
  REGION 1..317: note = SOX-2
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 74 - protein, 454 aa, synthetic construct
  REGION 1..454: note = c-MYC
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 75 - protein, 454 aa, synthetic construct
  REGION 1..454: note = OMOMYC
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAETQKLISEIDLLRKQNEQLKHKLEQLRNSCA

SEQ ID NO: 76 - protein, 513 aa, synthetic construct
  REGION 1..513: note = KLF4
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQGQSRGFVARAGEPCVCWPHFGTHGMMLTPPSSPLELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 77 - protein, 281 aa, synthetic construct
  REGION 1..281: note = NANOG
MTSKEDGKAAPGEERRRSPLDHLPPPANSNKPLTPFSIEDILNKPSVRRSYSLCGAAHLLAAADKHAQGGLPLAGRALLSQTSPLCALEELASKTFKGLEVSVLQAAEGRDGMTIFGQRQTPKKRRKSRTAFTNHQIYELEKRFLYQKYLSPADRDQIAQQLGLTNAQVITWFQNRRAKLKRDLEEMKADVESAKKLGPSGQMDIVALAELEQNSEATAGGGGGCGRAKSRPGSPVLPPGAPKAPGAGALQLSPASPLTDQPASSQDCSEDEEDEEIDVDD

SEQ ID NO: 78 - protein, 209 aa, synthetic construct
  REGION 1..209: note = protein lin-28
MGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQN

SEQ ID NO: 79 - protein, 347 aa, synthetic construct
  REGION 1..347: note = Asparaginase II
EFFKKTALAALVMGFSGAALALPNITILATGGTIAGGGDSATKSNYTVGKVGVENLVNAVPQLKDIANVKGEQVVNIGSQDMNDNVWLTLAKKINTDCDKTDGFVITHGTDTMEETAYFLDLTVKCDKPVVMVGAMRPSTSMSADGPFNLYNAVVTAADKASANRGVLVVMNDTVLDGRDVTKTNTTDVATFKSVNYGPLGYIHNGKIDYQRTPARKHTSDTPFDVSKLNELPKVGIVYNYANASDLPAKALVDAGYDGIVSAGVGNGNLYKSVFDTLATAAKTGTAVVRSSRVPTGATTQDAEVDDAKYGFVASGTLNPQKARVLLQLALTQTKDPQQIQQIFNQY

SEQ ID NO: 80 - DNA, 57 bp, synthetic construct
  source 1..57: mol_type = other DNA
  misc_feature 1..57: note = PT2A
gccacaaatttcagcctgctgaagcaggccggcgacgtggaagaaaatcctggacct

SEQ ID NO: 81 - protein, 19 aa, synthetic construct
  REGION 1..19: note = PT2A
ATNFSLLKQAGDVEENPGP

SEQ ID NO: 82 - protein, 202 aa, synthetic construct
  REGION 1..202: note = Self-cleavable domain
GSSCITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSHAT

SEQ ID NO: 83 - DNA, 606 bp, synthetic construct
  source 1..606: mol_type = other DNA
  misc_feature 1..606: note = Self-cleavable domain
ggcagcagctgtattacaggcgacgctctggtggctctgcctgagggcgagtctgttagaatcgccgacattgtgccaggcgccagacctaacagcgacaacgccatcgatctgaaggtgctggacagacacggcaaccctgtgctggccgatagactgtttcacagcggagagcaccccgtgtacacagtgcgaacagtggaaggcctgagagtgaccggaaccgccaatcatcctctgctgtgcctggtggatgtggccggtgttcctacactgctgtggaagctgatcgacgagatcaagcccggcgactacgccgtgattcagagaagcgccttcagcgtggactgcgccggatttgccagaggcaagcctgagtttgcccctaccacatacacagtgggagtgcctggactcgtgcggtttctggaagcccaccacagagatcccgacgctcaggccattgccgatgagctgacagacggcagattctactacgccaaggtggcctctgtgaccgatgctggtgtccagcctgtgtactctctgagagtggacacagccgaccacgccttcatcaccaatggctttgtgtcccacgccacc

SEQ ID NO: 84 - protein, 454 aa, synthetic construct
  REGION 1..454: note = Self-cleavable domain
CFAKGTNVLMADGSIECIENIEVGNKVMGKDGRPREVIKLPRGRETMYSVVQKSQHRAHKSDSSREVPELLKFTCNATHELVVRTPRSVRRLSRTIKGVEYFEVITFEMGQKKAPDGRIVELVKEVSKSYPISEGPERANELVESYRKASNKAYFEWTIEARDLSLLGSHVRKATYQTYAPILYENDHFFDYMQKSKFHLTIEGPKVLAYLLGLWIGDGLSDRATFSVDSRDTSLMERVTEYAEKLNLCAEYKDRKEPQVAKTVNLYSKVVRGNGIRNNLNTENPLWDAIVGLGFLKDGVKNIPSFLSTDNIGTRETFLAGLIDSDGYVTDEHGIKATIKTIHTSVRDGLVSLARSLGLVVSVNAEPAKVDMNVTKHKISYAIYMSGGDVLLNVLSKCAGSKKFRPAPAAAFARECRGFYFELQELKEDDYYGITLSDDSDHQFLLGSQVVVHA

SEQ ID NO: 85 - DNA, 1362 bp, synthetic construct
  source 1..1362: mol_type = other DNA
  misc_feature 1..1362: note = Self-cleavable domain
tgctttgccaagggcaccaatgtgctgatggccgacggcagcatcgagtgcatcgagaacatcgaagtgggcaacaaagtgatgggcaaagacggcagacccagggaagtgatcaagctgcccagaggccgggaaaccatgtacagcgtggtgcagaagtcccagcacagagcccacaagagcgacagcagcagagaagtgcctgagctgctgaagttcacctgtaacgccacacacgagctggtcgtgcggacacctagatctgtgcggagactgagccggaccatcaaaggcgtggaatactttgaagtcatcaccttcgagatgggccagaagaaggcccctgacggcagaatcgtggaactggtcaaagaggtgtccaagagctaccccatcagcgagggacctgagagggccaatgagctggtggaaagctaccggaaggccagcaacaaggcctacttcgagtggaccatcgaggccagagatctgagcctgctgggatctcatgtgcgcaaggccacctaccagacatacgcccctatcctgtacgagaacgaccacttcttcgactacatgcagaaaagcaagttccacctgacaatcgagggccccaaggtgctggcctatctgctcggactgtggatcggagatggcctgagcgatagagccaccttcagcgtggacagcagagacaccagcctgatggaaagagtgaccgagtacgccgagaagctgaacctgtgcgccgagtacaaggaccggaaagaaccccaggtggcaaagaccgtgaacctgtacagcaaggtcgtcagaggcaacggcatcagaaacaacctgaacaccgagaatcctctgtgggacgctatcgtcggcctgggctttctgaaggacggcgtgaagaatatccccagcttcctgagcaccgacaacatcggaaccagagagacattcctggccggcctgatcgactccgatggctacgtgacagatgagcacggcatcaaggccacaatcaagaccatccacaccagcgtcagagatggactggtgtccctggctagaagcctgggacttgtggtgtccgtgaatgccgagcctgccaaggtggacatgaacgtgaccaagcacaagatcagctacgccatctacatgtctggcggcgacgtgctgctgaacgtgctgtctaaatgtgccggcagcaagaagttcagacccgctcctgctgccgccttcgccagagaatgtagaggcttttacttcgagctgcaagagctgaaagaggacgactactacggcatcaccctgagcgacgacagcgaccaccaatttctgctgggaagccaggtggtggtgcatgct

SEQ ID NO: 86 - protein, 441 aa, synthetic construct
  REGION 1..441: note = Self-cleavable domain
ALAEGTRIFDPVTGTTHRIEDVVDGRKPIHVVAAAKDGTLHARPVVSWFDQGTRDVIGLRIAGGAILWATPDHKVLTEYGWRAAGELRKGDRVAQPRRFDGFGDSAPIPADHARLLGYLIGDGRDGWVGGKTPINFINVQRALIDDVTRIAATLGCAAHPQGRISLAIAHRPGERNGVADLCQQAGIYGKLAWEKTIPNWFFEPDIAADIVGNLLFGLFESDGWVSREQTGALRVGYTTTSEQLAHQIHWLLLRFGVGSTVRDYDPTQKRPSIVNGRRIQSKRQVFEVRISGMDNVTAFAESVPMWGPRGAALIQAIPEATQGRRRGSQATYLAAEMTDAVLNYLDERGVTAQEAAAMIGVASGDPRGGMKQVLGASRLRRDRVQALADALDDKFLHDMLAEELRYSVIREVLPTRRARTFGLEVEELHTLVAEGVVVHNC

SEQ ID NO: 87 - DNA, 1323 bp, synthetic construct
  source 1..1323: mol_type = other DNA
  misc_feature 1..1323: note = Self-cleavable domain
gctcttgccgagggcaccagaatcttcgatcctgtgaccggcaccacacaccggatcgaggatgtggtggatggcagaaagcccatccatgtggtggccgctgccaaggatggaaccctgcatgccagacctgtggtgtcttggtttgaccagggcacaagagatgtgatcggcctgagaattgccggcggagccattctgtgggccacacctgatcacaaggtgctgacagagtacggctggcgagctgctggggaactgagaaagggcgatagagtggcccagcctagaagattcgacggcttcggagacagcgcccctattcctgccgatcacgctagactgctgggctacctgatcggcgacggaagagatggatgggtcggaggcaagacccctatcaacttcatcaacgtgcagcgggccctgatcgacgatgtgaccagaattgctgccacactgggctgcgccgctcatcctcaaggcagaatctctctggctatcgcccacagacctggcgagagaaatggcgtggcagatctgtgtcagcaggccggcatctatggaaagctggcctgggagaaaacaatccccaactggttcttcgagcccgacattgccgccgacatcgtgggcaatctgctgttcggcctgtttgagtccgatggctgggtgtccagagaacagacaggcgctctgagagtgggctacaccacaacatctgagcagctggcccaccagatccactggctgctgctgagatttggcgtgggctctaccgtgcgggactacgaccctacacagaaaagacccagcatcgtgaacggcagacggatccagtccaagagacaggtgttcgaagtgcggatcagcggcatggacaacgtgacagcctttgccgagagcgtgccaatgtggggacctagaggtgccgctctgatccaggccattcctgaggctacacagggcagaagaagaggcagccaggccacatatctggccgccgaaatgacagacgccgtgctgaactacctggacgagaggggagtgacagcccaagaggccgctgctatgattggagtggcttccggcgatcctagaggcggcatgaagcaagtgctgggagcttcccggctgcggagagatagagttcaggctctggccgacgctctggacgacaagttcctgcatgatatgctggccgaggaactgcggtacagcgtgatcagagaggtgctgcctaccagacgggccagaacctttggcctggaagtggaagaactgcacaccctggtggctgaaggcgtggtggtgcataattgc

(19) United States
(12) Patent Application Publication
(10) Pub. No.: US 2025/0312464 A1
(43) Pub. Date: Oct. 9, 2025

(54) REVERSIBLE LOADING OF PROTEINS IN THE LUMEN OF EXTRACELLULAR VESICLES

(71) Applicants: CILOA, Montpellier (FR); JERID, Saint-André-de-Sangonis (FR)

(72) Inventors: Zaïne El Abiddine Robert MAMOUN, Saint-André-de-Sangonis (FR); Bernadette Nadine TRENTIN, Montpellier (FR); Etienne LOURDIN-DE FILIPPIS, Montpellier (FR)

(73) Assignees: CILOA, Montpellier (FR); JERID, Saint-André-de-Sangonis (FR)

(21) Appl. No.: 18/867,581

(22) PCT Filed: May 19, 2023
(86) PCT No.: PCT/EP2023/063497

§ 371 Date: Nov. 20, 2024

Related U.S. Application Data
(60) Provisional application No. 63/344,296, filed on May 20, 2022.

(51) Int. Cl. A61K 47/64; A61K 47/69; C07K 14/36

(52) U.S. Cl. CPC A61K 47/64; A61K 47/6911; C07K 14/36; C07K 2319/00

(57) ABSTRACT

The present invention relates to fusion polypeptides comprising a sub-membrane targeting domain, a protein of interest or a functionally or structurally active fragment thereof, and a peptide interacting with the Endosomal Sorting Complexes Required for Transport (ESCRT) cellular machinery, and the use of said fusion polypeptides in methods of targeting a protein of interest in the lumen of an extracellular vesicle. The present invention also relates to extracellular vesicles comprising said fusion polypeptides, and their use for treating or preventing diseases.

### FIELD OF INVENTION

The present invention relates to the field of therapeutic extracellular vesicles, in particular of therapeutic exosomes.

### BACKGROUND OF INVENTION

The use of extracellular vesicles (EVs) as therapeutic vectors presents a major interest when developing new biodrugs. Indeed, EVs can deliver proteins or nucleic acids of interest to a specific target cell, tissue or organ.

Loading EVs with these proteins or nucleic acids of interest is however challenging, and methods have been described to load proteins inside EVs. These methods are based on the modification of EVs-producing cells, or on the direct loading of EVs by physical or chemical methods (Ferreira et al., 2022. Crit Rev Oncol Hematol. 172:103628).

Here, the Inventors offer new methods to load proteins inside EVs. Two strategies were developed: (1) targeting of proteins of interest to the inner membrane of EVs via membrane anchorage; and (2) loading of proteins of interest in the lumen of EVs via a reversible interaction between (i) a carrier protein [e.g., streptavidin] anchored in the inner membrane of EVs according to strategy (1) and the protein of interest fused to a carrier-interacting peptide or protein [e.g., streptavidin-binding peptide].

### SUMMARY

The present invention relates to a fusion polypeptide comprising, from N-terminal to C-terminal:
(i) a sub-membrane targeting domain,
(ii) optionally, a linker,
(iii) a protein of interest or a functionally or structurally active fragment thereof,
(iv) optionally, a linker, and
(v) a peptide interacting with the Endosomal Sorting Complexes Required for Transport (ESCRT) cellular machinery.

In one embodiment, the sub-membrane targeting domain comprises or consists of an amino acid sequence (M)-G-X₁-X₂-X₃-X₄-X₅, wherein X₁, X₂, X₃ and X₄ independently from each other denote any amino acid residue, X₅ denote a basic amino acid residue, and (M) denotes an initiator methionine which, when located at the N-terminal extremity of the fusion polypeptide, can be removed in vivo by post-translation processing; optionally the sub-membrane targeting domain further comprises a basic patch comprising or consisting of several basic amino acid residues.

In one embodiment, the sub-membrane targeting domain comprises a myristic acid linked to a glycine residue, preferably the myristic acid is linked to the glycine residue at position 2 of the amino acid sequence (M)-G-X₁-X₂-X₃-X₄-X₅.

In one embodiment, the peptide interacting with the ESCRT cellular machinery comprises an amino acid sequence having one, two or three YxxL and/or DYxxL motif(s) (SEQ ID NO: 14), and one, two, three or four PxxP motif(s); preferably the peptide interacting with the ESCRT cellular machinery comprises an amino acid sequence having three YxxL and/or DYxxL motifs (SEQ ID NO: 14), and four PxxP motifs, more preferably the peptide interacting with the ESCRT cellular machinery comprises or consists of the amino acid sequence with SEQ ID NO: 38 or a variant thereof.

In one embodiment, the protein of interest is a therapeutic protein. In one embodiment, the protein of interest is selected from the group comprising or consisting of nuclear proteins, enzymes, antibodies (or fragments thereof), nanobodies and reporter proteins.

In one embodiment, the protein of interest as disclosed hereinabove is fused to at least one protein, at least one peptide or to at least one protein domain.

In one embodiment, the protein of interest as disclosed hereinabove is fused to a reporter protein.

In one embodiment, the protein of interest as disclosed hereinabove is fused to at least one peptide or at least one protein domain, that is self-cleavable.

In one embodiment, the at least one peptide comprises or consists of the self-cleavable PT2A (porcine teschovirus 1 2A) peptide. In one embodiment, the at least one protein domain comprises or consists of a self-cleavable domain derived from Mycobacterium xenopi gyrA protein, a self-cleavable domain derived from Saccharomyces cerevisiae VMA1 and/or a self-cleavable domain derived from Mycobacterium tuberculosis Recombinase A, more preferably said at least one protein domain is selected among domains with SEQ ID NO: 82, SEQ ID NO: 84, and SEQ ID NO: 86.

In one embodiment, the protein of interest is streptavidin or a fragment thereof, wherein the fragment of streptavidin retains its ability to bind to a streptavidin-binding peptide (SBP) and to biotin.

The present invention also relates to a method of targeting a protein of interest in the lumen of an extracellular vesicle, comprising contacting an extracellular vesicle-producing cell with the fusion polypeptide as described hereinabove or with a nucleic acid encoding said fusion polypeptide.

In one embodiment, the method comprises the steps of:
contacting an extracellular vesicle-producing cell with the fusion polypeptide as described hereinabove or with a nucleic acid encoding said fusion polypeptide;
culturing the extracellular vesicle-producing cell in a suitable culture medium for a time sufficient to allow extracellular vesicle production; and
recovering the extracellular vesicles produced by the extracellular vesicle-producing cell.

The present invention also relates to a population of extracellular vesicles comprising, in their lumen, the fusion polypeptide as described hereinabove; optionally the population of extracellular vesicles is obtainable by the method as described hereinabove.

The present invention also relates to a method of reversibly targeting a protein of interest in the lumen of an extracellular vesicle, comprising contacting an extracellular vesicle-producing cell with:
the fusion polypeptide with streptavidin or a fragment thereof as described hereinabove or a nucleic acid encoding said fusion polypeptide, and
a fusion polypeptide comprising (i) the protein of interest or a functionally or structurally active fragment thereof and (ii) a streptavidin-binding peptide (SBP), or a nucleic acid encoding said fusion polypeptide.

In one embodiment, the method comprises the steps of:
contacting an extracellular vesicle-producing cell with:
   the fusion polypeptide with streptavidin or a fragment thereof as described hereinabove or a nucleic acid encoding said fusion polypeptide, and
   the fusion polypeptide comprising (i) the protein of interest or a functionally or structurally active fragment thereof and (ii) the streptavidin-binding peptide (SBP), or the nucleic acid encoding said fusion polypeptide;
culturing the extracellular vesicle-producing cell in a suitable culture medium for a time sufficient to allow extracellular vesicle production; and
recovering the extracellular vesicles produced by the extracellular vesicle-producing cell.

In one embodiment, the protein of interest is a therapeutic protein. In one embodiment, the protein of interest is selected from the group comprising or consisting of nuclear proteins, enzymes, antibodies (or fragments thereof), nanobodies and reporter proteins.

In one embodiment, the protein of interest as disclosed hereinabove is fused to at least one protein, at least one peptide, or a at least one protein domain.

In one embodiment, the protein of interest as disclosed hereinabove is fused to a reporter protein.

In one embodiment, the protein of interest as disclosed hereinabove is fused to at least one peptide or at least one protein domain, that is self-cleavable.

In one embodiment, the at least one peptide comprises or consists of the self-cleavable PT2A (porcine teschovirus 1 2A) peptide. In one embodiment, the at least one protein domain comprises or consists of a self-cleavable domain derived from Mycobacterium xenopi gyrA protein, a self-cleavable domain derived from Saccharomyces cerevisiae VMA1 and/or a self-cleavable domain derived from Mycobacterium tuberculosis Recombinase A, more preferably said at least one protein domain is selected among domains with SEQ ID NO: 82, SEQ ID NO: 84, and SEQ ID NO: 86.

In one embodiment, the targeting of the protein of interest in the lumen of the extracellular vesicle is reversed by addition of biotin. In one embodiment, the protein of interest is released from the fusion polypeptide with streptavidin or a fragment thereof by addition of biotin or a structural analog thereof.

The present invention also relates to a population of extracellular vesicles comprising, in their lumen, the fusion polypeptide with streptavidin or a fragment thereof as described hereinabove and a fusion polypeptide comprising (i) a protein of interest or a functionally or structurally active fragment thereof and (ii) a streptavidin-binding peptide (SBP); optionally the population of extracellular vesicles is obtainable by the method as described hereinabove.

In one embodiment, the streptavidin-binding peptide (SBP) as disclosed hereinabove comprises or consists of the sequence with SEQ ID NO: 41 or a fragment thereof. In one embodiment, the streptavidin-binding peptide (SBP) comprises or consists of the sequence with SEQ ID NO: 42.

The present invention also relates to the population of extracellular vesicles as described hereinabove, for use as a drug.

The present invention also relates to the population of extracellular vesicles as described hereinabove, for use in preventing and/or treating a disease selected from the group consisting of cancer, genetic lysosomal diseases, diabetes, loss of function diseases, inflammation, infectious diseases, acquired immunodeficiencies, aging, and neurological diseases.

### Definitions

In the present invention, the following terms have the following meanings.

“About”, preceding a figure encompasses plus or minus 10%, or less, of the value of said figure. It is to be understood that the value to which the term “about” refers is itself also specifically, and preferably, disclosed.

“Enzyme” refers to a protein that act as a biological catalyst by accelerating chemical reactions. Enzymes may be classified according to their enzyme activity, such as, for example, EC1 for Oxidoreductases, EC2 for Transferases, EC3 for Hydrolases, EC4 for Lyases, EC5 for Isomerases, EC6 for Ligases, or EC7 for Translocases, according to the nomenclature developed by The International Union of Biochemistry and Molecular Biology. Enzymes may be found, for example, in micro-organisms including bacteria and yeasts, in plants or in animals.

“Exosome” refers to an extracellular vesicle that is produced in the endosomal compartment of eukaryotic cells (Théry et al., 2018. J Extracell Vesicles. 7(1):1535750; Yáñez-Mõ et al., 2015. J Extracell Vesicles. 4:27066; van Niel et al., 2018. Nat Rev Mol Cell Biol. 19(4):213-228). Typically, exosomes harbor at their surface the CD81, CD9, CD63 and tetraspanin-8 markers.

“Extracellular vesicles” refers to any vesicle composed of a lipid bilayer that is naturally released from a cell and comprises a cytosolic fraction of said cell. This expression in particular includes vesicles secreted into the extracellular space, i.e., “exosomes”.

“Global alignment” refers to alignment that aligns two sequences from beginning to end, aligning each letter in each sequence only once. An alignment is produced, regardless of whether or not there is similarity or identity between the sequences. For example, 50% sequence identity based on global alignment means that in an alignment of the full sequence of two compared sequences, each of 100 nucleotides or amino acid residues in length, 50% of the residues are the same. It is understood that global alignment can also be used in determining sequence identity even when the length of the aligned sequences is not the same. The differences in the terminal ends of the sequences will be taken into account in determining sequence identity, unless the “no penalty for end gaps” is selected. Generally, a global alignment is used on sequences that share significant similarity over most of their length. Exemplary algorithms for performing global alignment include the Needleman-Wunsch algorithm (Needleman & Wunsch, 1970. J Mol Biol. 48(3):443-53). Exemplary programs and software for performing global alignment are publicly available and include the Global Sequence Alignment Tool available at the National Center for Biotechnology Information (NCBI) website (http://ncbi.nlm.nih.gov), and the program available at deepc2.psi.iastate.edu/aat/align/align.html.

“Identity” or “sequence identity”: refers to the number of identical or similar nucleotides or amino acid residues in a comparison between a test and a reference sequence. Sequence identity can be determined by sequence alignment of nucleic acid or amino acid sequences to identify regions of similarity or identity. For purposes herein, sequence identity is generally determined by alignment to identify identical nucleotides or amino acid residues. The alignment can be local or global. Matches, mismatches and gaps can be identified between compared sequences. Gaps are null nucleotides or amino acid residues inserted between the residues of aligned sequences so that identical or similar characters are aligned. Generally, there can be internal and terminal gaps. When using gap penalties, sequence identity can be determined with no penalty for end gaps (e.g., terminal gaps are not penalized). Alternatively, sequence identity can be determined without taking into account gaps as

$$\frac{number of identical positions}{length of the total aligned sequence} \times 100.$$

For purposes herein, sequence identity can be determined by standard alignment algorithm programs used with default gap penalties established by each supplier. Default parameters for the GAP program can include:
a unary comparison matrix (containing a value of 1 for identities and 0 for non-identities) and the weighted comparison matrix of Gribskov & Burgess (1986. Nucleic Acids Res. 14(16):6745-63), as described by Schwartz & Dayhoff (1979. Matrices for detecting distant relationships. In Dayhoff (Ed.), Atlas of protein sequences. 5:353-358. Washington, DC: National Biomedical Research Foundation);
a penalty of 3.0 for each gap and an additional 0.10 penalty for each symbol in each gap; and
no penalty for end gaps.

“Linker” or “spacer” interchangeably refer to an amino acid sequence, typically a synthetic amino acid sequence, that connects or links two peptide or polypeptide sequences together. Linkers typically connect two peptide or polypeptide sequences via peptide bonds. Linkers are well-known in the art; see, e.g., Chen et al., 2013 (Adv Drug Deliv Rev. 65(10):1357-1369) or Klein et al., 2014 (Protein Eng Des Sel. 27(10):325-330), the content of which is incorporated herein by reference. Examples of suitable linkers include so-called “GS linkers” or “Gly-Ser linkers”, i.e., amino acid sequences essentially consisting of glycine (G) and serine (S) residues, and usually—but not always—comprising two or more repeats of a peptide motif. GS linkers are well-know and widely used in the art, in particular for their flexibility properties. In some embodiments, the GS linker comprises or consists of an amino acid sequence (GₓS)_{y} or (SGₓ)_{y}, wherein x ranges from 1 to 5 or more, such as 1, 2, 3, 4, 5 or more; and y ranges from 1 to 8 or more, such as 1, 2, 3, 4, 5, 6, 7, 8 or more. In some embodiments, the GS linker with amino acid sequence (GₓS)_{y} or (SGₓ)_{y} can further comprise one or several additional G and/or S residues in N-terminal and/or in C-terminal. In some embodiments, the GS linker comprises or consists of an amino acid sequence (GS)_{y}, (GGS)_{y} (SEQ ID NO: 1), (GGGS)_{y} (SEQ ID NO: 2), (GGGGS)_{y} (SEQ ID NO: 3), or (GGGGGS)_{y} (SEQ ID NO: 4), wherein y ranges from 1 to 8 or more, such as 1, 2, 3, 4, 5, 6, 7, 8 or more. Another example of suitable linker includes so-called “glycine linkers”, i.e., amino acid sequences essentially consisting of glycine (G) residues. Glycine linkers are well-know and widely used in the art, in particular for their flexibility properties. In some embodiments, the glycine linker comprises or consists of an amino acid sequence (G)_{z}, wherein z ranges from 1 to 10 or more, such as 1, 2, 3, 4, 5, 6, 7, 8, 9, 10 or more.

“Local alignment” refers to an alignment that aligns two sequence, but only aligns those portions of the sequences that share similarity or identity. Hence, a local alignment determines if sub-segments of one sequence are present in another sequence. If there is no similarity, no alignment will be returned. Local alignment algorithms include BLAST or Smith-Waterman algorithm (Smith & Waterman, 1981. Adv Appl Math. 2(4):482-9). For example, 50% sequence identity based on local alignment means that in an alignment of the full sequence of two compared sequences of any length, a region of similarity or identity of 100 nucleotides or amino acid residues in length has 50% of the residues that are the same in the region of similarity or identity.

“Loss of function diseases” refer to diseases caused by the impairment of one protein, with potentially distributed consequences. For example, in such diseases, a mutation may result in a gene product having less or no function.

“Nanobodies” refer to antibody-derived therapeutic proteins that contain the unique structural and functional properties of naturally-occurring heavy chain antibodies (Muyldermans, 2013. Annu Rev Biochem. 82:775-97). These heavy chain antibodies may contain a single variable domain (VHH) and two constant domains (CH2 and CH3).

“Neurological diseases” refer to any disease of the nervous system. Examples of neurological diseases include, for example, neurodegenerative diseases, injuries to the brain or spinal cord, stroke, seizure disorders, brain cancer, and neurological diseases due to infection.

“Reporter protein” refers to a protein encoded by a reporter gene, usually driven by a promoter. The reporter gene is a nucleic acid sequence encoding for easily assayed proteins. For example, the use of a fluorescent reporter protein as a protein of interest in a fusion polypeptide according to the present invention allows to observe the location and trafficking of organelles, vesicles or, of proteins of interest when a fluorescent protein is fused to the proteins of interest, in live cells and tissues. Examples of reporter proteins include, without limitation, β-galactosidase, luciferase (e.g. Nanoluc luciferase), and fluorescent proteins such as, for example, green fluorescent protein (GFP), DsRed, Cyan fluorescent protein (CFP) or yellow fluorescent protein (EYFP).

“Ribosomal protein” relates to proteins comprising the structural parts of the ribosome. These proteins are found in the small ribosomal subunits (RPSs) or in the large ribosomal subunit (RPLs).

“Streptavidin-binding peptide” (SBP) refer to peptides which bind streptavidin. In one embodiment, said SBP bind streptavidin with a dissociation constant less than about 1000 μm, 100 μm, 10 μM, 5 μM, 1 μM, 100 nM, 50 nM, 25 nM, or less than about 10 nM.

“Sub-membrane targeting domain” or “membrane targeting domain” or “membrane recruitment domain” are used interchangeably to refer to a domain capable of, in a cell and in particular in a eukaryotic cell (e.g., in an extracellular vesicle-producing cell), to anchor itself to a cell membrane and/or a vesicular membrane without being inserted into said membrane, said anchoring being achieved by means of one or more anchoring molecule(s) and/or by interactions (e.g., electrostatic interactions) between the sub-membrane targeting domain and the membrane. In some embodiments, the sub-membrane targeting domain is capable of binding to, or interacting with, the inner surface of the cell membrane (i.e., the cytoplasmic side of the cell membrane) and/or with the inner surface of vesicular membranes (i.e., the lumen side of the vesicular membrane).

“Nuclear protein” relates to proteins that are carried into and out of the nuclei through the nuclear pore complex by nucleocytoplasmic transport receptors. Such proteins include, for example, histone and non-histone proteins.

### DETAILED DESCRIPTION

The present invention relates to a fusion polypeptide comprising, from N-terminal to C-terminal:
a sub-membrane targeting domain,
optionally, a linker,
a protein of interest or a functionally or structurally active fragment thereof,
optionally, a linker, and
a peptide interacting with the Endosomal Sorting Complexes Required for Transport (ESCRT) cellular machinery.

According to the invention, the fusion polypeptide comprises a sub-membrane targeting domain.

In some embodiments, the sub-membrane targeting domain is sufficient to allow the fusion polypeptide to be anchored to the lipid bilayer of cellular or vesicular membranes, preferably via one or more anchoring molecules and/or through interactions such as electrostatic interactions.

Hence, due to its presence in the fusion polypeptide, the sub-membrane targeting domain allows the fusion polypeptide, when expressed in a cell, to be anchored to (or anchored in) a cell or vesicular membrane, without the fusion polypeptide being inserted into said membrane.

By “anchoring molecule”, it is meant any molecule capable of being inserted into at least one layer of the lipid bilayer of a cell or vesicular membrane. The anchoring molecule may be, e.g., a lipid or lipid-containing molecule. The sub-membrane targeting domain is then said to be “lipid-anchored”.

In some embodiments, the anchoring molecule comprises or consists of one or more lipids or lipid-containing molecules, said lipids comprising a hydrophobic carbon chain which allows them to encapsulate in the lipid bilayer of a cell or vesicular membrane.

In some embodiments, the lipids are fatty acids, including, without limitation, myristic acid, palmitic acid, and isoprenoid (such as, e.g., geranyl-geranyl and farnesyl).

In some embodiments, the anchoring molecule is linked to the sub-membrane targeting domain by a covalent bond.

In some embodiments, the anchoring molecule is linked to the sub-membrane targeting domain through a glycine (e.g., in the case of a myristic acid), cysteine or serine amino acid residue of the sub-membrane targeting domain. This link may be through an amide or thioester bond.

In some embodiments, the sub-membrane targeting domain is that of an extrinsic membrane protein or is a variant of the sub-membrane targeting domain of an extrinsic membrane protein.

In some embodiments, the sub-membrane targeting domain comprises or consists of a consensus sequence allowing the attachment (e.g., by acylation or by prenylation) of a fatty acid, and in particular of myristic acid, palmitic acid, or isoprenoid (such as, e.g., geranyl-geranyl and farnesyl).

In some embodiments, the sub-membrane targeting domain comprises or consists of the consensus sequence (M)-G-X₁-X₂-X₃-X₄-X₅, wherein X₁, X₂, X₃ and X₄ independently from each other denote any amino acid residue, X₅ denote a basic amino acid residue, and (M) denotes an initiator methionine which, when located at the N-terminal extremity of the fusion polypeptide, can be removed in vivo by post-translation processing.

In some embodiments, the glycine residue at position 2 (if the initiator methionine is present, else position 1 when the initiator methionine is removed) is linked to an anchoring molecule as defined above, preferably to a lipid, more preferably to a myristic acid.

In some embodiments, X₁ is selected from the group comprising or consisting of C, S and L; and/or X₂ is selected from the group comprising or consisting of S, I, V, M and L; and/or X₃ is selected from the group comprising or consisting of K, Q, H, F, C and S, preferably X₃ is K; and/or X₄ is selected from the group comprising or consisting of S and C; and/or X₅ is selected from the group comprising or consisting of K, R and H; preferably X₅ is K.

In some embodiments, the sub-membrane targeting domain may further comprise several basic amino acid residues, in particular several amino acid residues selected from the group comprising or consisting of K, R and H. These basic amino acid residues are organized in what is known in the art as a “basic patch”; it is readily understandable that this basic patch does not necessarily consists of contiguous basic amino acid residues, but can span 5, 10, 15 or more amino acid residues within which several basic amino acid residues are scattered. By “several”, it is meant at least 2, and preferably at least 3 or more. These basic amino acid residues may in particular be involved in interactions with lipids of cell or vesicular membranes, especially with choline and derivative thereof (e.g., with phosphatidylcholine), and thus make it possible to increase the affinity of the sub-membrane targeting domain for these membranes.

In some embodiments, the basic patch may be located at least partially in the consensus sequence (M)-G-X₁-X₂-X₃-X₄-X₅ defined above, and/or outside this consensus sequence. In some embodiments, the basic patch may start from residue X₅ of the consensus sequence (M)-G-X₁-X₂-X₃-X₄-X₅, or alternatively may start after residue X₅ of this consensus sequence, such as from residue n+1, n+2 or n+3 after residue X₅.

Thus, in some embodiments, the sub-membrane targeting domain:
comprises or consists of an amino acid sequence chosen among:
   (M)-G-X-X-K-S/C-K-X-K (SEQ ID NO: 5), and
   (M)-G-X-X-K-S/C-K-X-K-X-X-X-X-R-R-R (SEQ ID NO: 6),
   wherein X denotes any amino acid residue, and wherein (M) denotes an initiator methionine which, when located at the N-terminal extremity of the chimeric polypeptide, can be removed in vivo by post-translation processing; or
is a variant of any of these sequences, said variant retaining the ability of the sub-membrane targeting domain to be anchored in the lipid bilayer of a cell or vesicular membrane.

In some embodiments, the sub-membrane targeting domain is derived from a protein of the Src family of proteins. Examples of such proteins include, without limitation, Src, Yes, Lyn, Fyn, Lck, Blk, Fgr, Hck and Yrk proteins (Resh, 1994. Cell. 76(3):411-413), and more particularly the N-terminal portion of one of these proteins, such as 15, 16, 17, 18, 19, 20, 21, 22, 23, 24 or 25 N-terminal amino acid residues of one of these proteins.

In some embodiments, the sub-membrane targeting domain is derived from the c-Src or v-Src protein, and preferably from the c-Src.

Alternatively, the sub-membrane targeting domain may be derived from other acylated proteins, such as, e.g., viral capsid proteins, including, without limitation, the human immunodeficiency virus (HIV) MA protein, or filovirus proteins.

In some embodiments, the sub-membrane targeting domain is derived from a Src protein.

In some embodiments, the sub-membrane targeting domain is derived from a Src protein and comprises or consists of one of the following amino acid sequences:

(SEQ ID NO: 7)
(M)GSSKSKPKDPSQRRR,
(SEQ ID NO: 8)
(M)GSSKSKPKDPSQRRRKSR,
(SEQ ID NO: 9)
(M)GSSKSKPKDPSQRRRKSRGPGG,

a variant of any of these sequences, said variant retaining the ability of the sub-membrane targeting domain to be anchored in the lipid bilayer of a cell or vesicular membrane;
   wherein (M) denotes an initiator methionine which, when located at the N-terminal extremity of the chimeric polypeptide, can be removed in vivo by post-translation processing.

In some embodiments, a variant of any of these three amino acid sequences comprises an amino acid sequence sharing at least 70% of global sequence identity, preferably at least 75%, 80%, 85%, 90%, 95%, 96%, 97%, 98%, 99% or more of global sequence identity with any one of these amino acid sequences.

Additionally or alternatively, a variant of any of these three amino acid sequences comprises an amino acid sequence sharing at least 70% of local sequence identity, preferably at least 75%, 80%, 85%, 90%, 95%, 96%, 97%, 98%, 99% or more of local sequence identity with any one of these amino acid sequences.

In some embodiments, the sub-membrane targeting domain is derived from a Src protein as defined above, and further comprises one or more anchoring molecules as defined above, in particular, comprises a myristic acid (in the form of a myristyl moiety) linked to the glycine residue at position 2 (if the initiator methionine is present, else position 1 when the initiator methionine is removed).

According to the invention, the fusion polypeptide comprises a protein of interest or a functionally or structurally active fragment thereof.

In some embodiments, the protein of interest is any protein or functionally and/or structurally active fragment thereof. In particular, the protein of interest may be a therapeutic protein.

A “therapeutic protein” as used herein is typically a peptide or a protein, which is beneficial for the treatment or prophylaxis of any inherited or acquired disease or which improves the condition of a subject. In particular, therapeutic proteins may play a role in the modification and repair of genetic deficiencies, in the destruction of cancer cells or of pathogens and pathogen-infected cells, and/or in the treatment or prevention of various diseases including immune system disorders such as auto-immune diseases, metabolic or endocrine disorders, diseases of the nervous system, diseases of the circulatory system, diseases of the respiratory system, diseases of the digestive system, diseases of the skin and subcutaneous tissue, diseases of the musculoskeletal system and connective tissue, diseases of the genitourinary system, etc., independently if they are inherited or acquired.

In the context of this invention, the term “therapeutic protein” typically refers to both peptides and proteins. It may also refer to a polypeptide or polyprotein comprising a therapeutic protein as defined herein. For instance, the term may refer to a polypeptide comprising the therapeutic protein fused, preferably in N-terminal or C-terminal, to a further amino acid sequence which is not derived from the therapeutic protein.

In some embodiments, the therapeutic protein may be a precursor protein, which is processed in vivo or in vitro to yield the final, active therapeutic protein.

In some embodiments, the protein of interest is an enzyme, a transcription factor, a trans-dominant negative oncoprotein mutant (such as, e.g., Omomyc), an RNA-binding protein (such as, e.g., phage MS2 capsid protein), an RNA-guided nuclease (such as, e.g., Cas9), an antibody or a binding-fragment thereof (including scFvs, di-scFvs, tri-scFvs, F(ab′)₂, Fab′, nanobodies, microantibodies, intrabodies, and the like), an antibody mimetic (including affibodies, affilins, affimers, affitin, alphabodies, anticalins, avimers, DARPins, Kunitz domain peptides, monobodies, nanoCLAMPs, and the like), signaling molecules- or signal transducing molecules-binding proteins, cytoplasmic kinase receptors and functionally active domains thereof, FK506- or FK1012 dimer-binding proteins (such as, e.g., FKBP12).

In some embodiments, the protein of interest is any protein, except a ribosomal protein. Examples of ribosomal proteins include, without limitation, eukaryotic translation initiation factors of the eIF family, such as, for example, eIF4E, eIF2, eIF1A, eIF5B and eIF1.

In some embodiments, the protein of interest is selected from the group comprising or consisting of nuclear proteins, enzymes, antibodies (or fragments thereof), nanobodies and reporter proteins. In some embodiments, the protein of interest is selected from the group comprising or consisting of nuclear proteins, enzymes and reporter proteins.

In one embodiment, the protein of interest is a nuclear protein, such as, for example, a transcription factor. In one embodiment, the protein of interest in an enzyme, such as, for example, a bacterial enzyme. In one embodiment, the protein of interest is a reporter protein, such as, for example, a fluorescent reporter protein. In one embodiment, the protein of interest is an antibody or a fragment thereof. In one embodiment, the protein of interest is a nanobody.

In some embodiments, the protein of interest is a transcription factor. In one embodiment, the protein of interest is a transcription factor selected from the group comprising or consisting of OCT4 (octamer-binding transcription factor 4, also known as POU5F1 (POU domain, class 5, transcription factor 1)), SOX-2 (SRY-Box Transcription Factor 2), c-MYC, OMOMYC, KLF4 (Krueppel-like factor 4), homeobox protein NANOG (also known as transcription factor LBX1), or protein lin-28.

In one embodiment, the protein of interest is a reporter protein, such as, for example, GFP, eGFP or Nanoluc.

In one embodiment, the protein of interest is an enzyme, such as, for example, asparaginase II.

In one embodiment, the protein of interest is OCT4. In one embodiment, the protein of interest comprises or consists of the sequence with SEQ ID NO: 67, which is encoded by a nucleic sequence with SEQ ID NO: 68.

SEQ ID NO: 67
MAGHLASDFAFSPPPGGGGDGPGGPEPGWVDPRTWLSFQGPPGGPGIGP
GVGPGSEVWGIPPCPPPYEFCGGMAYCGPQVGVGLVPQGGLETSQPEGE
AGVGVESNSDGASPEPCTVTPGAVKLEKEKLEQNPEESQDIKALQKELE
QFAKLLKQKRITLGYTQADVGLTLGVLFGKVFSQTTICRFEALQLSFKN
MCKLRPLLQKWVEEADNNENLQEICKAETLVQARKRKRTSIENRVRGNL
ENLFLQCPKPTLQQISHIAQQLGLEKDVVRVWFCNRRQKGKRSSSDYAQ
REDFEAAGSPFSGGPVSFPLAPGPHFGTPGYGSPHFTALYSSVPFPEGE
AFPPVSVTTLGSPMHSN

SEQ ID NO: 68
ATGGCTGGACATCTGGCCTCCGACTTCGCCTTCTCTCCACCACCTGGCG
GAGGCGGAGATGGACCAGGTGGACCTGAACCTGGATGGGTTGACCCTAG
AACCTGGCTGAGCTTTCAGGGACCTCCTGGCGGACCTGGAATTGGACCT
GGTGTTGGCCCTGGCTCTGAAGTGTGGGGAATCCCTCCTTGTCCTCCAC
CTTACGAGTTCTGTGGCGGCATGGCCTACTGTGGCCCTCAAGTTGGAGT
TGGCCTGGTGCCTCAAGGCGGCCTGGAAACATCTCAGCCTGAGGGCGAA
GCTGGCGTGGGCGTCGAGTCTAATTCTGATGGCGCCTCTCCTGAGCCTT
GCACCGTTACACCTGGCGCCGTGAAGCTGGAAAAAGAGAAACTGGAACA
GAACCCCGAGGAAAGCCAGGACATCAAGGCCCTGCAGAAAGAGCTGGAA
CAGTTCGCCAAGCTGCTGAAGCAGAAGCGGATCACCCTGGGCTACACAC
AGGCTGATGTGGGCCTGACACTGGGCGTGCTGTTTGGCAAGGTGTTCAG
CCAGACCACCATCTGTAGATTCGAAGCCCTGCAGCTGAGCTTCAAGAAC
ATGTGCAAGCTGCGGCCCCTGCTGCAGAAATGGGTTGAAGAGGCCGACA
ACAACGAGAACCTGCAAGAGATCTGCAAGGCCGAGACACTGGTGCAGGC
CCGGAAGAGAAAGAGAACCAGCATCGAGAACAGAGTGCGGGGCAACCTG
GAAAACCTGTTCCTGCAGTGCCCCAAGCCTACACTGCAGCAGATCAGCC
ACATTGCCCAGCAGCTGGGACTCGAAAAGGACGTCGTCAGAGTGTGGTT
CTGCAACCGGCGGCAGAAGGGCAAGAGAAGCAGCAGCGATTACGCCCAG
AGAGAGGACTTTGAGGCCGCTGGCAGTCCTTTTTCTGGCGGCCCTGTGT
CCTTTCCTCTGGCTCCTGGACCTCACTTTGGCACACCTGGCTATGGCAG
CCCTCACTTCACAGCCCTGTACAGCAGCGTGCCCTTTCCAGAAGGCGAG
GCCTTTCCTCCTGTGTCCGTGACAACACTGGGCAGCCCTATGCACAGCA
ACTGA

In one embodiment, the protein of interest is eGFP. In one embodiment, the protein of interest comprises or consists of the sequence with SEQ ID NO: 69, which is encoded by a nucleic sequence with SEQ ID NO: 70.

SEQ ID NO: 69
MVSKGEELFTGVVPILVELDGDVNGHKFSVSGEGEGDATYGKLTLKFIC
TTGKLPVPWPTLVTTLTYGVQCFSRYPDHMKQHDFFKSAMPEGYVQERT
IFFKDDGNYKTRAEVKFEGDTLVNRIELKGIDFKEDGNILGHKLEYNYN
SHNVYIMADKQKNGIKVNFKIRHNIEDGSVQLADHYQQNTPIGDGPVLL
PDNHYLSTQSALSKDPNEKRDHMVLLEFVTAAGITLGMDELYK

SEQ ID NO: 70
ATGGTGTCCAAGGGCGAAGAACTGTTCACCGGCGTGGTGCCCATTCTGG
TGGAACTGGATGGGGATGTGAACGGCCACAAGTTCAGCGTTAGCGGAGA
AGGCGAAGGCGACGCCACATACGGAAAGCTGACCCTGAAGTTCATCTGC
ACCACCGGCAAGCTGCCTGTGCCTTGGCCTACACTGGTCACAACCCTGA
CATACGGCGTGCAGTGCTTCAGCAGATACCCCGACCATATGAAGCAGCA
CGACTTCTTCAAGAGCGCCATGCCTGAGGGCTACGTGCAAGAGCGGACC
ATCTTCTTTAAGGACGACGGCAACTACAAGACCAGGGCCGAAGTGAAGT
TCGAGGGCGACACCCTGGTCAACCGGATCGAGCTGAAGGGCATCGACTT
CAAAGAGGACGGCAACATCCTGGGCCACAAGCTTGAGTACAACTACAAC
AGCCACAACGTGTACATCATGGCCGACAAGCAGAAAAACGGCATCAAAG
TGAACTTCAAGATCCGGCACAACATCGAGGACGGCTCTGTGCAGCTGGC
CGATCACTACCAGCAGAACACACCCATCGGAGATGGCCCTGTGCTGCTG
CCCGATAACCACTACCTGAGCACACAGAGCGCCCTGAGCAAGGACCCCA
ACGAGAAGAGGGATCACATGGTGCTGCTGGAATTCGTGACCGCCGCTGG
CATCACACTCGGCATGGATGAGCTGTACAAG

In one embodiment, the protein of interest is NanoLuc. In one embodiment, the protein of interest comprises or consists of the sequence with SEQ ID NO: 71, which is encoded by a nucleic sequence with SEQ ID NO: 72.

SEQ ID NO: 71
MVFTLEDFVGDWRQTAGYNLDQVLEQGGVSSLFQNLGVSVTPIQRIVL
SGENGLKIDIHVIIPYEGLSGDQMGQIEKIFKVVYPVDDHHFKVILHY
GTLVIDGVTPNMIDYFGRPYEGIAVFDGKKITVTGTLWNGNKIIDERL
INPDGSLLFRVTINGVTGWRLCERILA

SEQ ID NO: 72
ATGGTGTTCACCCTGGAAGATTTCGTCGGCGACTGGCGGCAGACAGCCGGC
TATAATCTGGACCAGGTGCTGGAACAAGGCGGCGTGTCCAGCCTGTTTCAG
AACCTGGGAGTGTCCGTGACACCCATCCAGAGAATCGTGCTGAGCGGCGAG
AACGGCCTGAAGATCGACATCCACGTGATCATCCCTTACGAGGGCCTGTCC
GGCGATCAGATGGGACAGATCGAGAAGATCTTTAAGGTGGTGTACCCCGTG
GACGACCACCACTTCAAAGTGATCCTGCACTACGGCACCCTGGTCATCGAT
GGCGTGACCCCTAACATGATCGACTACTTCGGCAGACCCTACGAGGGAATC
GCCGTGTTCGACGGCAAGAAAATCACCGTGACCGGCACACTGTGGAACGGC
AACAAGATCATCGACGAGCGGCTGATCAACCCCGATGGCAGCCTGCTGTTC
AGAGTGACCATCAACGGCGTGACAGGATGGCGGCTGTGCGAGAGAATTCTT
GCC

In one embodiment, the protein of interest is SOX-2. In one embodiment, the protein of interest comprises or consists of the sequence with SEQ ID NO: 73.

SEQ ID NO: 73
MYNMMETELKPPGPQQTSGGGGGNSTAAAAGGNQKNSPDRVKRPMNAFM
VWSRGQRRKMAQENPKMHNSEISKRLGAEWKLLSETEKRPFIDEAKRLR
ALHMKEHPDYKYRPRRKTKTLMKKDKYTLPGGLLAPGGNSMASGVGVGA
GLGAGVNQRMDSYAHMNGWSNGSYSMMQDQLGYPQHPGLNAHGAAQMQP
MHRYDVSALQYNSMTSSQTYMNGSPTYSMSYSQQGTPGMALGSMGSVVK
SEASSSPPVVTSSSHSRAPCQAGDLRDMISMYLPGAEVPEPAAPSRLHM
SQHYQSGPVPGTAINGTLPLSHM

In one embodiment, the protein of interest is c-MYC. In one embodiment, the protein of interest comprises or consists of the sequence with SEQ ID NO: 74.

SEQ ID NO: 74
MDFFRVVENQQPPATMPLNVSFTNRNYDLDYDSVQPYFYCDEEENFYQQ
QQQSELQPPAPSEDIWKKFELLPTPPLSPSRRSGLCSPSYVAVTPFSLR
GDNDGGGGSFSTADQLEMVTELLGGDMVNQSFICDPDDETFIKNIIIQD
CMWSGFSAAAKLVSEKLASYQAARKDSGSPNPARGHSVCSTSSLYLQDL
SAAASECIDPSVVFPYPLNDSSSPKSCASQDSSAFSPSSDSLLSSTESS
PQGSPEPLVLHEETPPTTSSDSEEEQEDEEEIDVVSVEKRQAPGKRSES
GSPSAGGHSKPPHSPLVLKRCHVSTHQHNYAAPPSTRKDYPAAKRVKLD
SVRVLRQISNNRKCTSPRSSDTEENVKRRTHNVLERQRRNELKRSFFAL
RDQIPELENNEKAPKVVILKKATAYILSVQAEEQKLISEEDLLRKRREQ
LKHKLEQLRNSCA

In one embodiment, the protein of interest is OMOMYC. In one embodiment, the protein of interest comprises or consists of the sequence with SEQ ID NO: 75.

SEQ ID NO: 75
MDFFRVVENQQPPATMPLNVSFTNRNYDLDYDSVQPYFYCDEEENFYQQ
QQQSELQPPAPSEDIWKKFELLPTPPLSPSRRSGLCSPSYVAVTPFSLR
GDNDGGGGSFSTADQLEMVTELLGGDMVNQSFICDPDDETFIKNIIIQD
CMWSGFSAAAKLVSEKLASYQAARKDSGSPNPARGHSVCSTSSLYLQDL
SAAASECIDPSVVFPYPLNDSSSPKSCASQDSSAFSPSSDSLLSSTESS
PQGSPEPLVLHEETPPTTSSDSEEEQEDEEEIDVVSVEKRQAPGKRSES
GSPSAGGHSKPPHSPLVLKRCHVSTHQHNYAAPPSTRKDYPAAKRVKLD
SVRVLRQISNNRKCTSPRSSDTEENVKRRTHNVLERQRRNELKRSFFAL
RDQIPELENNEKAPKVVILKKATAYILSVQAETQKLISEIDLLRKQNEQ
LKHKLEQLRNSCA

In one embodiment, the protein of interest is KLF4. In one embodiment, the protein of interest comprises or consists of the sequence with SEQ ID NO: 76.

SEQ ID NO: 76
MRQPPGESDMAVSDALLPSFSTFASGPAGREKTLRQAGAPNNRWREELS
HMKRLPPVLPGRPYDLAAATVATDLESGGAGAACGGSNLAPLPRRETEE
FNDLLDLDFILSNSLTHPPESVAATVSSSASASSSSSPSSSGPASAPST
CSFTYPIRAGNDPGVAPGGTGGGLLYGRESAPPPTAPFNLADINDVSPS
GGFVAELLRPELDPVYIPPQQPQPPGGGLMGKFVLKASLSAPGSEYGSP
SVISVSKGSPDGSHPVVVAPYNGGPPRTCPKIKQEAVSSCTHLGAGPPL
SNGHRPAAHDFPLGRQLPSRTTPTLGLEEVLSSRDCHPALPLPPGFHPH
PGPNYPSFLPDQMQPQVPPLHYQGQSRGFVARAGEPCVCWPHFGTHGMM
LTPPSSPLELMPPGSCMPEEPKPKRGRRSWPRKRTATHTCDYAGCGKTY
TKSSHLKAHLRTHTGEKPYHCDWDGCGWKFARSDELTRHYRKHTGHRPF
QCQKCDRAFSRSDHLALHMKRHF

In one embodiment, the protein of interest is NANOG. In one embodiment, the protein of interest comprises or consists of the sequence with SEQ ID NO: 77.

SEQ ID NO: 77
MTSKEDGKAAPGEERRRSPLDHLPPPANSNKPLTPFSIEDILNKPSVRR
SYSLCGAAHLLAAADKHAQGGLPLAGRALLSQTSPLCALEELASKTFKG
LEVSVLQAAEGRDGMTIFGQRQTPKKRRKSRTAFTNHQIYELEKRFLYQ
KYLSPADRDQIAQQLGLTNAQVITWFQNRRAKLKRDLEEMKADVESAKK
LGPSGQMDIVALAELEQNSEATAGGGGGCGRAKSRPGSPVLPPGAPKAP
GAGALQLSPASPLTDQPASSQDCSEDEEDEEIDVDD

In one embodiment, the protein of interest is protein lin-28. In one embodiment, the protein of interest comprises or consists of the sequence with SEQ ID NO: 78.

SEQ ID NO: 78
MGSVSNQQFAGGCAKAAEEAPEEAPEDAARAADEPQLLHGAGICKWFNV
RMGFGFLSMTARAGVALDPPVDVFVHQSKLHMEGFRSLKEGEAVEFTFK
KSAKGLESIRVTGPGGVFCIGSERRPKGKSMQKRRSKGDRCYNCGGLDH
HAKECKLPPQPKKCHFCQSISHMVASCPLKAQQGPSAQGKPTYFREEEE
EIHSPTLLPEAQN

In one embodiment, the protein of interest is asparaginase II. In one embodiment, the protein of interest comprises or consists of the sequence with SEQ ID NO: 79.

SEQ ID NO: 79
EFFKKTALAALVMGFSGAALALPNITILATGGTIAGGGDSATKSNYTVG
KVGVENLVNAVPQLKDIANVKGEQVVNIGSQDMNDNVWLTLAKKINTDC
DKTDGFVITHGTDTMEETAYFLDLTVKCDKPVVMVGAMRPSTSMSADGP
FNLYNAVVTAADKASANRGVLVVMNDTVLDGRDVTKTNTTDVATFKSVN
YGPLGYIHNGKIDYQRTPARKHTSDTPFDVSKLNELPKVGIVYNYANAS
DLPAKALVDAGYDGIVSAGVGNGNLYKSVFDTLATAAKTGTAVVRSSRV
PTGATTQDAEVDDAKYGFVASGTLNPQKARVLLQLALTQTKDPQQIQQI
FNQY

In one embodiment, the protein of interest comprises a protein of interest as disclosed herein fused to at least one protein, at least one peptide or to at least one protein domain. Said at least one protein, at least one peptide or at least one protein domain may be fused upstream or downstream of the protein of interest.

In one embodiment, the protein of interest comprises a protein of interest as disclosed herein fused to two elements being selected among proteins, peptides and protein domains. Said proteins, peptides and protein domains may be fused upstream of the protein of interest, downstream of the protein of interest or both upstream and downstream of the protein of interest (i.e. one upstream and one downstream).

In one embodiment, the protein of interest comprises a protein of interest as disclosed herein fused to a reporter protein, which enables to trace the protein of interest within the cell or tissues. Examples of reporter proteins are disclosed hereinabove.

In one embodiment, the protein of interest comprises a protein of interest as disclosed herein fused to at least one peptide or at least one protein domain, that can be self-cleaved.

In one embodiment, the at least one peptide comprises or consists of the self-cleavable PT2A (porcine teschovirus 1 2A) peptide. In one embodiment, the at least one peptide comprises or consists of the peptide with SEQ ID NO: 81, encoded by a nucleic acid with SEQ ID NO: 80.

SEQ ID NO: 80
GCCACAAATTTCAGCCTGCTGAAGCAGGCCGGCGACGTGGAAGAAAATC
CTGGACCT

SEQ ID NO: 81
ATNFSLLKQAGDVEENPGP

In one embodiment, the at least one protein domain is self-cleaved in reducing chemical conditions.

In one embodiment, the at least one protein domain that is self-cleaved in reducing chemical conditions comprises or consists of a domain derived from Mycobacterium xenopi gyrA protein. In one embodiment, the at least one protein domain comprises or consists of the domain with SEQ ID NO: 82, encoded by a nucleic acid with SEQ ID NO: 83. In one embodiment, the protein of interest comprises a protein of interest as disclosed herein fused to a self-cleavable domain derived from Mycobacterium xenopi gyrA protein, preferably wherein the protein of interest is fused upstream of the domain.

SEQ ID NO: 82
GSSCITGDALVALPEGESVRIADIVPGARPNSDNAIDLKVLDRHGNPVL
ADRLFHSGEHPVYTVRTVEGLRVTGTANHPLLCLVDVAGVPTLLWKLID
EIKPGDYAVIQRSAFSVDCAGFARGKPEFAPTTYTVGVPGLVRFLEAHH
RDPDAQAIADELTDGRFYYAKVASVTDAGVQPVYSLRVDTADHAFITNG
FVSHAT

SEQ ID NO: 83
GGCAGCAGCTGTATTACAGGCGACGCTCTGGTGGCTCTGCCTGAGGGCGAG
TCTGTTAGAATCGCCGACATTGTGCCAGGCGCCAGACCTAACAGCGACAAC
GCCATCGATCTGAAGGTGCTGGACAGACACGGCAACCCTGTGCTGGCCGAT
AGACTGTTTCACAGCGGAGAGCACCCCGTGTACACAGTGCGAACAGTGGAA
GGCCTGAGAGTGACCGGAACCGCCAATCATCCTCTGCTGTGCCTGGTGGAT
GTGGCCGGTGTTCCTACACTGCTGTGGAAGCTGATCGACGAGATCAAGCCC
GGCGACTACGCCGTGATTCAGAGAAGCGCCTTCAGCGTGGACTGCGCCGGA
TTTGCCAGAGGCAAGCCTGAGTTTGCCCCTACCACATACACAGTGGGAGTG
CCTGGACTCGTGCGGTTTCTGGAAGCCCACCACAGAGATCCCGACGCTCAG
GCCATTGCCGATGAGCTGACAGACGGCAGATTCTACTACGCCAAGGTGGCC
TCTGTGACCGATGCTGGTGTCCAGCCTGTGTACTCTCTGAGAGTGGACACA
GCCGACCACGCCTTCATCACCAATGGCTTTGTGTCCCACGCCACC

In one embodiment, the at least one protein domain that is cleaved in reducing chemical conditions comprises or consists of a domain derived from Saccharomyces cerevisiae VMA1. In one embodiment, the at least one protein domain comprises or consists of the domain with SEQ ID NO: 84, encoded by a nucleic acid with SEQ ID NO: 85. In one embodiment, the protein of interest comprises a protein of interest as disclosed herein fused to a self-cleavable domain derived from Saccharomyces cerevisiae VMA1, preferably wherein the protein of interest is fused downstream of the domain.

SEQ ID NO: 84
CFAKGTNVLMADGSIECIENIEVGNKVMGKDGRPREVIKLPRGRETMYSV
VQKSQHRAHKSDSSREVPELLKFTCNATHELVVRTPRSVRRLSRTIKGVE
YFEVITFEMGQKKAPDGRIVELVKEVSKSYPISEGPERANELVESYRKAS
NKAYFEWTIEARDLSLLGSHVRKATYQTYAPILYENDHFFDYMQKSKFHL
TIEGPKVLAYLLGLWIGDGLSDRATFSVDSRDTSLMERVTEYAEKLNLCA
EYKDRKEPQVAKTVNLYSKVVRGNGIRNNLNTENPLWDAIVGLGFLKDGV
KNIPSFLSTDNIGTRETFLAGLIDSDGYVTDEHGIKATIKTIHTSVRDGL
VSLARSLGLVVSVNAEPAKVDMNVTKHKISYAIYMSGGDVLLNVLSKCAG
SKKFRPAPAAAFARECRGFYFELQELKEDDYYGITLSDDSDHQFLLGSQV
VVHA

SEQ ID NO: 85
TGCTTTGCCAAGGGCACCAATGTGCTGATGGCCGACGGCAGCATCGAGTGC
ATCGAGAACATCGAAGTGGGCAACAAAGTGATGGGCAAAGACGGCAGACC
CAGGGAAGTGATCAAGCTGCCCAGAGGCCGGGAAACCATGTACAGCGTGGT
GCAGAAGTCCCAGCACAGAGCCCACAAGAGCGACAGCAGCAGAGAAGTGC
CTGAGCTGCTGAAGTTCACCTGTAACGCCACACACGAGCTGGTCGTGCGGA
CACCTAGATCTGTGCGGAGACTGAGCCGGACCATCAAAGGCGTGGAATACT
TTGAAGTCATCACCTTCGAGATGGGCCAGAAGAAGGCCCCTGACGGCAGAA
TCGTGGAACTGGTCAAAGAGGTGTCCAAGAGCTACCCCATCAGCGAGGGAC
CTGAGAGGGCCAATGAGCTGGTGGAAAGCTACCGGAAGGCCAGCAACAAG
GCCTACTTCGAGTGGACCATCGAGGCCAGAGATCTGAGCCTGCTGGGATCT
CATGTGCGCAAGGCCACCTACCAGACATACGCCCCTATCCTGTACGAGAAC
GACCACTTCTTCGACTACATGCAGAAAAGCAAGTTCCACCTGACAATCGAG
GGCCCCAAGGTGCTGGCCTATCTGCTCGGACTGTGGATCGGAGATGGCCTG
AGCGATAGAGCCACCTTCAGCGTGGACAGCAGAGACACCAGCCTGATGGAA
AGAGTGACCGAGTACGCCGAGAAGCTGAACCTGTGCGCCGAGTACAAGGA
CCGGAAAGAACCCCAGGTGGCAAAGACCGTGAACCTGTACAGCAAGGTCGT
CAGAGGCAACGGCATCAGAAACAACCTGAACACCGAGAATCCTCTGTGGGA
CGCTATCGTCGGCCTGGGCTTTCTGAAGGACGGCGTGAAGAATATCCCCAG
CTTCCTGAGCACCGACAACATCGGAACCAGAGAGACATTCCTGGCCGGCCT
GATCGACTCCGATGGCTACGTGACAGATGAGCACGGCATCAAGGCCACAAT
CAAGACCATCCACACCAGCGTCAGAGATGGACTGGTGTCCCTGGCTAGAAG
CCTGGGACTTGTGGTGTCCGTGAATGCCGAGCCTGCCAAGGTGGACATGAA
CGTGACCAAGCACAAGATCAGCTACGCCATCTACATGTCTGGCGGCGACGT
GCTGCTGAACGTGCTGTCTAAATGTGCCGGCAGCAAGAAGTTCAGACCCGC
TCCTGCTGCCGCCTTCGCCAGAGAATGTAGAGGCTTTTACTTCGAGCTGCA
AGAGCTGAAAGAGGACGACTACTACGGCATCACCCTGAGCGACGACAGCGA
CCACCAATTTCTGCTGGGAAGCCAGGTGGTGGTGCATGCT

In one embodiment, the at least one protein domain is self-cleaved in low pH conditions.

In one embodiment, the at least one protein domain that is cleaved in low pH conditions comprises or consists of a domain derived from Mycobacterium tuberculosis recombinase A. In one embodiment, the at least one protein domain comprises or consists of the domain with SEQ ID NO: 86, encoded by a nucleic acid with SEQ ID NO: 87. In one embodiment, the protein of interest comprises a protein of interest as disclosed herein fused to a self-cleavable domain derived from Mycobacterium tuberculosis recombinase A, preferably wherein the protein of interest is fused downstream of the domain.

SEQ ID NO: 86
ALAEGTRIFDPVTGTTHRIEDVVDGRKPIHVVAAAKDGTLHARPVVSWF
DQGTRDVIGLRIAGGAILWATPDHKVLTEYGWRAAGELRKGDRVAQPRR
FDGFGDSAPIPADHARLLGYLIGDGRDGWVGGKTPINFINVQRALIDDV
TRIAATLGCAAHPQGRISLAIAHRPGERNGVADLCQQAGIYGKLAWEKT
IPNWFFEPDIAADIVGNLLFGLFESDGWVSREQTGALRVGYTTTSEQLA
HQIHWLLLRFGVGSTVRDYDPTQKRPSIVNGRRIQSKRQVFEVRISGMD
NVTAFAESVPMWGPRGAALIQAIPEATQGRRRGSQATYLAAEMTDAVLN
YLDERGVTAQEAAAMIGVASGDPRGGMKQVLGASRLRRDRVQALADALD
DKFLHDMLAEELRYSVIREVLPTRRARTFGLEVEELHTLVAEGVVVHNC
SEQ ID NO: 87
GCTCTTGCCGAGGGCACCAGAATCTTCGATCCTGTGACCGGCACCACAC
ACCGGATCGAGGATGTGGTGGATGGCAGAAAGCCCATCCATGTGGTGGC
CGCTGCCAAGGATGGAACCCTGCATGCCAGACCTGTGGTGTCTTGGTTT
GACCAGGGCACAAGAGATGTGATCGGCCTGAGAATTGCCGGCGGAGCCA
TTCTGTGGGCCACACCTGATCACAAGGTGCTGACAGAGTACGGCTGGCG
AGCTGCTGGGGAACTGAGAAAGGGCGATAGAGTGGCCCAGCCTAGAAGA
TTCGACGGCTTCGGAGACAGCGCCCCTATTCCTGCCGATCACGCTAGAC
TGCTGGGCTACCTGATCGGCGACGGAAGAGATGGATGGGTCGGAGGCAA
GACCCCTATCAACTTCATCAACGTGCAGCGGGCCCTGATCGACGATGTG
ACCAGAATTGCTGCCACACTGGGCTGCGCCGCTCATCCTCAAGGCAGAA
TCTCTCTGGCTATCGCCCACAGACCTGGCGAGAGAAATGGCGTGGCAGA
TCTGTGTCAGCAGGCCGGCATCTATGGAAAGCTGGCCTGGGAGAAAACA
ATCCCCAACTGGTTCTTCGAGCCCGACATTGCCGCCGACATCGTGGGCA
ATCTGCTGTTCGGCCTGTTTGAGTCCGATGGCTGGGTGTCCAGAGAACA
GACAGGCGCTCTGAGAGTGGGCTACACCACAACATCTGAGCAGCTGGCC
CACCAGATCCACTGGCTGCTGCTGAGATTTGGCGTGGGCTCTACCGTGC
GGGACTACGACCCTACACAGAAAAGACCCAGCATCGTGAACGGCAGACG
GATCCAGTCCAAGAGACAGGTGTTCGAAGTGCGGATCAGCGGCATGGAC
AACGTGACAGCCTTTGCCGAGAGCGTGCCAATGTGGGGACCTAGAGGTG
CCGCTCTGATCCAGGCCATTCCTGAGGCTACACAGGGCAGAAGAAGAGG
CAGCCAGGCCACATATCTGGCCGCCGAAATGACAGACGCCGTGCTGAAC
TACCTGGACGAGAGGGGAGTGACAGCCCAAGAGGCCGCTGCTATGATTG
GAGTGGCTTCCGGCGATCCTAGAGGCGGCATGAAGCAAGTGCTGGGAGC
TTCCCGGCTGCGGAGAGATAGAGTTCAGGCTCTGGCCGACGCTCTGGAC
GACAAGTTCCTGCATGATATGCTGGCCGAGGAACTGCGGTACAGCGTGA
TCAGAGAGGTGCTGCCTACCAGACGGGCCAGAACCTTTGGCCTGGAAGT
GGAAGAACTGCACACCCTGGTGGCTGAAGGCGTGGTGGTGCATAATTGC

In one embodiment, the protein of interest as disclosed hereinabove is fused to at least one protein domain that is self-cleavable, preferably wherein said at least one protein domain is derived from Mycobacterium xenopi gyrA protein, Saccharomyces cerevisiae VMA1 and/or Mycobacterium tuberculosis Recombinase A.

In one embodiment, the protein of interest comprises a protein of interest as disclosed herein fused to two self-cleavable domains as disclosed herein. Said domains may be fused upstream of the protein of interest, downstream of the protein of interest or both upstream and downstream of the protein of interest (i.e. one upstream and one downstream).

In one embodiment, the protein of interest comprises a protein of interest as disclosed herein fused to two protein domains that are self-cleavable, wherein said protein domains are derived from Mycobacterium xenopi gyrA protein, Saccharomyces cerevisiae VMA1 and/or Mycobacterium tuberculosis Recombinase A, as disclosed herein. In one embodiment, the protein of interest comprises a protein of interest as disclosed herein fused to two protein domains that are self-cleavable, wherein said protein domains are selected among domains with SEQ ID NO: 82, SEQ ID NO: 84, and SEQ ID NO: 86.

In one embodiment, the protein of interest comprises a protein of interest as disclosed herein fused to two self-cleavable domains, said domains being i) one self-cleavable domain derived from Mycobacterium xenopi gyrA as disclosed herein, and i) one self-cleavable domain derived from Saccharomyces cerevisiae VMA1 protein or one self-cleavable domain derived from Mycobacterium tuberculosis Recombinase A, as disclosed herein. In one embodiment, the domain derived from Mycobacterium xenopi gyrA is fused downstream of the protein of interest. In one embodiment, the domain derived from Saccharomyces cerevisiae VMA1 protein or the domain derived from Mycobacterium tuberculosis Recombinase A is fused upstream of the protein of interest.

According to the invention, the fusion polypeptide comprises a peptide interacting with the Endosomal Sorting Complexes Required for Transport (ESCRT) cellular machinery, otherwise known as “Pilot Peptide”.

In some embodiments, the pilot peptide is capable of being addressed to extracellular vesicles, in particular to exosome vesicles, or to the cell compartment(s) involved in the formation of extracellular vesicles.

Pilot peptides which interact with ESCRT proteins have been described in granted patents EP 2 268 816 B1, and U.S. Pat. No. 9,546,371 B2, the relevant content of which is incorporated herein by reference.

In some embodiments, the pilot peptide comprises at least one YxxL motif, in which “x” represents any amino acid residue. In particular, it may comprise one, two or three YxxL motifs. The YxxL motif or one of the YxxL motifs of the pilot peptide may, for example, be YINL (SEQ ID NO: 10) or YSHL (SEQ ID NO: 11).

Alternatively or additionally, the pilot peptide may comprise at least one motif equivalent to a YxxL motif, for example, a YxxF motif, in which “x” represents any amino acid residue. The YxxF motif or one of the YxxF motifs of the pilot peptide may then be, for example, be YINF (SEQ ID NO: 12) or YSHF (SEQ ID NO: 13).

In some embodiments, the pilot peptide comprises a DYxxL motif (SEQ ID NO: 14), in which “x” represents any amino acid residue. The DYxxL motif (SEQ ID NO: 14) or one of the DYxxL motifs (SEQ ID NO: 14) of the pilot peptide may, for example, be DYINL (SEQ ID NO: 15).

Alternatively or additionally, the pilot peptide may comprise at least one motif equivalent to a DYxxL motif (SEQ ID NO: 14), for example, a DYxxF motif (SEQ ID NO: 16), in which “x” represents any amino acid residue. The DYxxF motif (SEQ ID NO: 16) or one of the DYxxF motifs (SEQ ID NO: 16) of the pilot peptide may then be, for example, be DYINF (SEQ ID NO: 17).

In some embodiments, the pilot peptide further comprises at least one PxxP motif, in which “x” represents any amino acid residue. In particular, it may comprise one, two, three or four PxxP motifs.

In some embodiments, the PxxP motif or one of the PxxP motifs of the pilot peptide may, for example, be PSAP (SEQ ID NO: 18) or PTAP (SEQ ID NO: 19).

In some embodiments, the pilot peptide comprises at least one YxxL motif or DYxxL motif (SEQ ID NO: 14), and at least one PxxP motif.

In some embodiments, the pilot peptide comprises or consists of an amino acid sequence having one, two or three YxxL and/or DYxxL motif(s) (SEQ ID NO: 14); and one, two, three or four PxxP motif(s).

In some embodiments, the pilot peptide comprises or consists of an amino acid sequence having three YxxL and/or DYxxL motifs (SEQ ID NO: 14), and four PxxP motifs.

In some embodiments, the YxxL motif or at least one of the YxxL motifs, when more than one, is located downstream, i.e., in a C-terminal position, with respect to the one or more PxxP motif(s).

Proteins having a pilot peptide comprising at least one YxxL motif include cellular proteins and viral proteins. In particular, these viral proteins are proteins of enveloped viruses, such as transmembrane glycoproteins of enveloped viruses, or herpesvirus proteins, e.g., the LMP2-A protein of the Epstein-Barr virus which comprises at least two YxxL motifs.

In some embodiments, the pilot peptide is that of a transmembrane glycoprotein of a retrovirus. In one embodiment, the pilot peptide may be that of a transmembrane glycoprotein of a retrovirus selected from the group comprising or consisting of bovine leukemia virus (BLV), human immunodeficiency virus (HIV) (such as, without limitation, HIV-1 or HIV-2), human T-cell leukemia virus (HTLV) (such as, without limitation, HTLV-1 or HTLV-2), and Mason-Pfizer monkey virus (MPMV).

In some embodiments, the pilot peptide comprises one of the following amino acid sequences:
PxxPxxxxPxxPxSxYxxLxPxxPExYxxLxPxxPDYxxL (SEQ ID NO: 20);
PxxPxₙPxxPxₙSxYxxLxₙPxxPExₙYxxLxₙPxxPDYxxL (SEQ ID NO: 21);
PxxPxxxxPxxPxSxYxxLxPxxPExYxxLxPxxPDYxxLxxxx (SEQ ID NO: 22); and
PxxPxₙPxxPxₙSxYxxLxₙPxxPExₙYxxLxₙPxxPDYxxLxxxx (SEQ ID NO: 23);
   in which “x” and “xₙ”, respectively, represent any amino acid residue and any one or several amino acid residue(s).

In some embodiments, the pilot peptide comprises one of the following amino acid sequences:
PxxPxxxxxxxxxxxxYxxL (SEQ ID NO: 24);
PxxPxxxxxxxxxxxDYxxL (SEQ ID NO: 25);
PxxPxxYxxxxxxxxxYxxL (SEQ ID NO: 26);
PxxPxxYxxxxxxxxDYxxL (SEQ ID NO: 27);
PxxPExYxxLxPxxPDYxxL (SEQ ID NO: 28);
PxxPxₙYxxL (SEQ ID NO: 29);
PxxPxₙDYxxL (SEQ ID NO: 30);
PxxPxₙYxₙYxxL (SEQ ID NO: 31);
PxxPxₙYxₙDYxxL (SEQ ID NO: 32);
PxxPExYxxLxₙPxxPDYxxL (SEQ ID NO: 33);
PxxPxxxxPxxPxxxYxxLxPxxPExYxxLxPxxPDYxxL (SEQ ID NO: 34);
PxxPxₙPxxPxₙYxxLxₙPxxPExₙYxxLxₙPxxPDYxxL (SEQ ID NO: 35);
PxxPxxxxPxxPxxxYxxLxPxxPExYxxLxPxxPDYxxLxxxx (SEQ ID NO: 36); and
PxxPxₙPxxPxₙYxxLxₙPxxPExₙYxxLxₙPxxPDYxxLxxxx (SEQ ID NO: 37);
   in which “x” and “xₙ”, respectively, represent any amino acid residue and any one or several amino acid residue(s).

In particular, “n” may be greater than or equal to 1 and less than 50. “n” may, in particular, have any value between 1 and 20.

In some embodiments, the pilot peptide comprises from 6 to 100 amino acid residues, in particular from 20 to 80, from 30 to 70, or from 40 to 60 amino acid residues, for example 40, 41, 42, 43, 44, 45, 46, 47, 48, 49 or 50 amino acid residues.

In some embodiments, the pilot peptide comprises or consists of the amino acid sequence APHFPEISFPPKPDSDYQALLPSAPEIYSHLSPTKPDYINLRPAP (SEQ ID NO: 38) or a variant thereof.

A variant of this amino acid sequence may comprise an amino acid sequence sharing at least 70% of global sequence identity, preferably at least 75%, 80%, 85%, 90%, 95%, 96%, 97%, 98%, 99% or more of global sequence identity with this amino acid sequence.

Additionally or alternatively, a variant of this amino acid sequence may comprise an amino acid sequence sharing at least 70% of local sequence identity, preferably at least 75%, 80%, 85%, 90%, 95%, 96%, 97%, 98%, 99% or more of local sequence identity with this amino acid sequence.

Preferably, a variant of this amino acid sequence retains at least one, two or three YxxL or DYxxL motif(s) (SEQ ID NO: 14), and one, two, three or four PxxP motifs.

Even more preferably, a variant of this amino acid sequence retains three YxxL and/or DYxxL motifs (SEQ ID NO: 14), and four PxxP motifs.

In some embodiments, the fusion polypeptide comprises, from N-terminal to C-terminal:
a sub-membrane targeting domain with amino acid sequence (M)-G-X₁-X₂-X₃-X₄-X₅, wherein X₁, X₂, X₃ and X₄ independently from each other denote any amino acid residue, X₅ denote a basic amino acid residue, and (M) denotes an initiator methionine which, when located at the N-terminal extremity of the fusion polypeptide, can be removed in vivo by post-translation processing,
optionally, a linker,
a protein of interest or a functionally or structurally active fragment thereof,
optionally, a linker, and
a peptide interacting with the Endosomal Sorting Complexes Required for Transport (ESCRT) cellular machinery, said peptide comprising an amino acid sequence having one, two or three YxxL and/or DYxxL motif(s) (SEQ ID NO: 14), and one, two, three or four PxxP motif(s).

In some embodiments, the fusion polypeptide comprises, from N-terminal to C-terminal:
a Src-derived sub-membrane targeting domain comprising a myristic acid (in the form of a myristyl moiety) linked to its glycine residue at position 2 (if the initiator methionine is present, else position 1 when the initiator methionine is removed),
optionally, a linker,
a protein of interest or a functionally or structurally active fragment thereof,
optionally, a linker, and
a peptide interacting with the Endosomal Sorting Complexes Required for Transport (ESCRT) cellular machinery, said peptide comprising an amino acid sequence having three YxxL and/or DYxxL motifs (SEQ ID NO: 14), and four PxxP motifs.

In some embodiments, the fusion polypeptide comprises, from N-terminal to C-terminal:
a Src-derived sub-membrane targeting domain with amino acid sequence MGSSKSKPKDPSQRRR (SEQ ID NO: 7), comprising a myristic acid (in the form of a myristyl moiety) linked to the glycine residue at position 2 (if the initiator methionine is present, else position 1 when the initiator methionine is removed),
optionally, a linker,
a protein of interest or a functionally or structurally active fragment thereof,
optionally, a linker, and
a peptide interacting with the Endosomal Sorting Complexes Required for Transport (ESCRT) cellular machinery, with amino acid sequence APHFPEISFPPKPDSDYQALLPSAPEIYSHLSPTKPDYINLRPAP (SEQ ID NO: 38).

In some embodiments, the fusion polypeptide comprises, from N-terminal to C-terminal:
a Src-derived sub-membrane targeting domain with amino acid sequence MGSSKSKPKDPSQRRRKSR (SEQ ID NO: 8), comprising a myristic acid (in the form of a myristyl moiety) linked to the glycine residue at position 2 (if the initiator methionine is present, else position 1 when the initiator methionine is removed),
optionally, a linker,
a protein of interest or a functionally or structurally active fragment thereof,
optionally, a linker, and
a peptide interacting with the Endosomal Sorting Complexes Required for Transport (ESCRT) cellular machinery, with amino acid sequence APHFPEISFPPKPDSDYQALLPSAPEIYSHLSPTKPDYINLRPAP (SEQ ID NO: 38).

In some embodiments, the fusion polypeptide comprises, from N-terminal to C-terminal:
a Src-derived sub-membrane targeting domain with amino acid sequence MGSSKSKPKDPSQRRRKSRGPGG (SEQ ID NO: 9), comprising a myristic acid (in the form of a myristyl moiety) linked to the glycine residue at position 2 (if the initiator methionine is present, else position 1 when the initiator methionine is removed),
optionally, a linker,
a protein of interest or a functionally or structurally active fragment thereof,
optionally, a linker, and
a peptide interacting with the Endosomal Sorting Complexes Required for Transport (ESCRT) cellular machinery, with amino acid sequence APHFPEISFPPKPDSDYQALLPSAPEIYSHLSPTKPDYINLRPAP (SEQ ID NO: 38).

The present invention also relates to a nucleic acid encoding a fusion protein of the present invention.

The fusion polypeptide described above is suitable for targeting a protein of interest in the lumen of an extracellular vesicle, as demonstrated by the Inventors in the EXAMPLES section.

The present invention relates thus to a method of targeting a protein of interest in the lumen of an extracellular vesicle.

According to this invention, the method comprises contacting an extracellular vesicle-producing cell (or a population of extracellular vesicle-producing cells) with the fusion polypeptide described above, or with a nucleic acid encoding this fusion polypeptide.

In some embodiments, the extracellular vesicle-producing cell (or a population of extracellular vesicle-producing cells) is contacted with a nucleic acid encoding the fusion polypeptide. This step may be carried out in particular by transfecting extracellular vesicle-producing cells with the nucleic acid.

The method can be performed in vivo, in vitro or ex vivo.

In some embodiments, the extracellular vesicle-producing cell is a HEK293 cell or a cell from a derivative cell line.

In some embodiments, the extracellular vesicle-producing cell is an adipocyte.

In some embodiments, the extracellular vesicle-producing cell is an immune cell, including, but not limited to, a mastocyte, a lymphocyte (such as, e.g., a T-cell or a B-cell), and a dendritic cell.

In some embodiments, the extracellular vesicle-producing cell is a stem cell, including, but not limited to, an embryonic stem cell, an adult stem cell (such as, e.g., a hematopoietic stem cell, a mammary stem cell, an intestinal stem cell, a mesenchymal stem cell, an adipocyte stem cell, an endothelial stem cell, a neural stem cell, an olfactory adult stem cell, or a neural crest stem cell), a cancer stem cell, an induced pluripotent stem cell (iPSC) and an induced stem cell (iSC).

The skilled artisan is aware of methods of obtaining embryonic stem cells, in particular human embryonic stem cells, which methods do not require embryo destruction; see, e.g., Chung et al., 2008. Cell Stem Cell. 2(2):113-117, which is the first of several publications disclosing such a method.

In some embodiments, it may be desirable to recover these extracellular vesicles into the lumen of which a protein of interest has been targeted. Accordingly, the method may comprise steps of:
contacting an extracellular vesicle-producing cell (or a population of extracellular vesicle-producing cells) with the fusion polypeptide described above, or with a nucleic acid encoding this fusion polypeptide;
culturing the extracellular vesicle-producing cell (or the population of such cells) in a suitable culture medium; and
recovering the extracellular vesicles produced by the extracellular vesicle-producing cell (or the population of such cells).

General means and methods for producing extracellular vesicles are well known in the art. See, e.g., Whitford & Guterstam, 2019. Future Med Chem. 11(10):1225-1236; Taylor & Shah, 2015. Methods. 87:3-10; Desplantes et al., 2017. Sci Rep. 7(1):1032.

In some embodiments, the step of culturing the extracellular vesicle-producing cell (or the population of such cells) in a suitable culture medium is carried out for a time sufficient to allow extracellular vesicle production.

Extracellular vesicles, as per their name, are produced by extracellular vesicle-producing cells and released in the culture supernatant.

In some embodiments, the culture medium is itself devoid of extraneous extracellular vesicles. For instance, the culture medium could be a serum-free medium, a medium supplemented with extracellular vesicle-depleted serum, or a medium supplemented with extracellular vesicle-depleted platelet lysate.

In some embodiments, the third step of recovering the extracellular vesicles comprises isolating or otherwise purifying these extracellular vesicles.

Isolating or purifying extracellular vesicles may comprise one or several substeps of clarification (such as, e.g., by centrifugation or by depth-filtration), filtration, ultra-filtration, diafiltration, size-exclusion purification and/or ion-exchange chromatography of the cell culture supernatant.

Thus, the present invention also relates to a method for producing extracellular vesicles comprising, in their lumen, the fusion polypeptide as described herein.

An exemplary protocol of this method is provided in the EXAMPLES section.

The present invention also relates to an extracellular vesicle (or a population of such extracellular vesicles), said extracellular vesicle comprising the fusion polypeptide described above.

According to the invention, the fusion polypeptide is located in the lumen of an extracellular vesicle. Still according to the invention, the fusion polypeptide is anchored or otherwise attached, via its sub-membrane targeting domain, to the inner extracellular vesicle membrane.

In some embodiments, the extracellular vesicle is a small extracellular vesicle.

In some embodiments, the extracellular vesicle is an exosome. Exosomes may have a diameter typically ranging from about 30 nm to about 150 nm, preferably from about 30 nm to about 120 nm, more preferably from about 40 nm to about 80 nm. In particular, exosomes may have a diameter ranging from about 30 nm to about 120 nm.

In some embodiments, the population of extracellular vesicles is monodisperse in aqueous solutions, preferably in a NaCl 0.9% aqueous solution and/or in PBS. By “monodisperse”, it is meant that the extracellular vesicles in the population of extracellular vesicles are substantially uniform in size. By “substantially uniform”, it is meant that the extracellular vesicles have a narrow distribution of sizes around an average size. In one embodiment, the extracellular vesicles in water and/or in PBS have sizes exhibiting a standard deviation of less than 100% with respect to their average size, such as less than 75%, 50%, 40%, 30%, 20%, 10%, or less than 5%.

A particular aspect of the present invention concerns a fusion polypeptide as described above, comprising from N-terminal to C-terminal:
a sub-membrane targeting domain (as described above),
optionally, a linker,
streptavidin or a fragment thereof, preferably wherein the fragment of streptavidin retains its ability to bind to a streptavidin-binding peptide (SBP) and to biotin,
optionally, a linker, and
a peptide interacting with the Endosomal Sorting Complexes Required for Transport (ESCRT) cellular machinery (as described above).

In the present disclosure, this particular fusion polypeptide is term “carrier protein”.

In some embodiments, this particular fusion polypeptide (“carrier protein”) comprises, from N-terminal to C-terminal:
a sub-membrane targeting domain with amino acid sequence (M)-G-X₁-X₂-X₃-S/C (SEQ ID NO: 39), wherein X₁, X₂, X₃ and X₄ independently from each other denote any amino acid residue, X₅ denote a basic amino acid residue, and (M) denotes an initiator methionine which, when located at the N-terminal extremity of the fusion polypeptide, can be removed in vivo by post-translation processing,
optionally, a linker,
streptavidin or a fragment thereof, preferably wherein the fragment of streptavidin retains its ability to bind to a streptavidin-binding peptide (SBP) and to biotin,
optionally, a linker, and
a peptide interacting with the Endosomal Sorting Complexes Required for Transport (ESCRT) cellular machinery, said peptide comprising an amino acid sequence having one, two or three YxxL and/or DYxxL motif(s) (SEQ ID NO: 14), and one, two, three or four PxxP motif(s).

In some embodiments, this particular fusion polypeptide (“carrier protein”) comprises, from N-terminal to C-terminal:
a Src-derived sub-membrane targeting domain comprising a myristic acid (in the form of a myristyl moiety) linked to its glycine residue at position 2 (if the initiator methionine is present, else position 1 when the initiator methionine is removed),
optionally, a linker,
streptavidin or a fragment thereof, preferably wherein the fragment of streptavidin retains its ability to bind to a streptavidin-binding peptide (SBP) and to biotin,
optionally, a linker, and
a peptide interacting with the Endosomal Sorting Complexes Required for Transport (ESCRT) cellular machinery, said peptide comprising an amino acid sequence having three YxxL and/or DYxxL motifs (SEQ ID NO: 14), and four PxxP motifs.

In some embodiments, this particular fusion polypeptide (“carrier protein”) comprises, from N-terminal to C-terminal:
a Src-derived sub-membrane targeting domain with amino acid sequence MGSSKSKPKDPSQRRR (SEQ ID NO: 7), comprising a myristic acid (in the form of a myristyl moiety) linked to the glycine residue at position 2 (if the initiator methionine is present, else position 1 when the initiator methionine is removed),
optionally, a linker,
streptavidin, without its signal peptide, with amino acid sequence

(SEQ ID NO: 40)
DPSKDSKAQVSAAEAGITGTWYNQLGSTFIVTAGADGALTGTYESAVGN
AESRYVLTGRYDSAPATDGSGTALGWTVAWKNNYRNAHSATTWSGQY
VGGAEARINTQWLLTSGTTEANAWKSTLVGHDTFTKVKPSAASIDAAKK
AGVNNGNPLDAVQQ,

optionally, a linker, and
a peptide interacting with the Endosomal Sorting Complexes Required for Transport (ESCRT) cellular machinery, with amino acid sequence APHFPEISFPPKPDSDYQALLPSAPEIYSHLSPTKPDYINLRPAP (SEQ ID NO: 38).

In some embodiments, this particular fusion polypeptide (“carrier protein”) comprises, from N-terminal to C-terminal:
a Src-derived sub-membrane targeting domain with amino acid sequence MGSSKSKPKDPSQRRRKSR (SEQ ID NO: 8), comprising a myristic acid (in the form of a myristyl moiety) linked to the glycine residue at position 2 (if the initiator methionine is present, else position 1 when the initiator methionine is removed),
optionally, a linker,
streptavidin, without its signal peptide, with amino acid sequence

(SEQ ID NO: 40)
DPSKDSKAQVSAAEAGITGTWYNQLGSTFIVTAGADGALTGTYESAVGN
AESRYVLTGRYDSAPATDGSGTALGWTVAWKNNYRNAHSATTWSGQY
VGGAEARINTQWLLTSGTTEANAWKSTLVGHDTFTKVKPSAASIDAAKK
AGVNNGNPLDAVQQ,

optionally, a linker, and
a peptide interacting with the Endosomal Sorting Complexes Required for Transport (ESCRT) cellular machinery, with amino acid sequence APHFPEISFPPKPDSDYQALLPSAPEIYSHLSPTKPDYINLRPAP (SEQ ID NO: 38).

In some embodiments, this particular fusion polypeptide (“carrier protein”) comprises, from N-terminal to C-terminal:
a Src-derived sub-membrane targeting domain with amino acid sequence MGSSKSKPKDPSQRRRKSRGPGG (SEQ ID NO: 9), comprising a myristic acid (in the form of a myristyl moiety) linked to the glycine residue at position 2 (if the initiator methionine is present, else position 1 when the initiator methionine is removed),
optionally, a linker,
streptavidin, without its signal peptide, with amino acid sequence

(SEQ ID NO: 40)
DPSKDSKAQVSAAEAGITGTWYNQLGSTFIVTAGADGALTGTYESAVGN
AESRYVLTGRYDSAPATDGSGTALGWTVAWKNNYRNAHSATTWSGQY
VGGAEARINTQWLLTSGTTEANAWKSTLVGHDTFTKVKPSAASIDAAKK
AGVNNGNPLDAVQQ,

optionally, a linker, and
a peptide interacting with the Endosomal Sorting Complexes Required for Transport (ESCRT) cellular machinery, with amino acid sequence APHFPEISFPPKPDSDYQALLPSAPEIYSHLSPTKPDYINLRPAP (SEQ ID NO: 38).

Accordingly, another particular aspect of the present invention concerns a method of targeting streptavidin or a fragment thereof in the lumen of an extracellular vesicle, comprising contacting an extracellular vesicle-producing cell (or a population of extracellular vesicle-producing cells) with the particular fusion polypeptide with streptavidin or a fragment thereof described above (“carrier protein”), or with a nucleic acid encoding this fusion polypeptide. Embodiments described above in relation with the method of targeting a protein of interest in the lumen of an extracellular vesicle apply mutatis mutandis to this particular aspect of the method.

Accordingly, another particular aspect of the present invention concerns an extracellular vesicle (or a population of such extracellular vesicles), said extracellular vesicle comprising the particular fusion polypeptide with streptavidin or a fragment thereof described above (“carrier protein”). Embodiments described above in relation with the extracellular vesicle or the population of such extracellular vesicles apply mutatis mutandis to this particular aspect of the extracellular vesicle or population of such extracellular vesicles.

The particular fusion polypeptide with streptavidin or a fragment thereof described above (“carrier protein”) is suitable for reversibly targeting a protein of interest in the lumen of an extracellular vesicle, as demonstrated by the Inventors in the EXAMPLES section.

The present invention relates thus to a method of reversibly targeting a protein of interest in the lumen of an extracellular vesicle.

By “reversibly”, it is meant that the targeting of the protein of interest to the lumen of an extracellular vesicle can be countermanded “on demand”, e.g., upon application of a biological, chemical or physical stimulus. Additionally or alternatively, the term “reversibly” is also used herein to refer to the release of the protein of interest after it has been targeted and “trapped” in the lumen of an extracellular vesicle “on demand”, e.g., upon application of a biological, chemical or physical stimulus.

According to this invention, the method comprises steps of contacting an extracellular vesicle-producing cell (or a population of extracellular vesicle-producing cells) with:
the particular fusion polypeptide with streptavidin or a fragment thereof described above (“carrier protein”), or with a nucleic acid encoding this fusion polypeptide, and
a fusion polypeptide comprising (i) the protein of interest or a functionally or structurally active fragment thereof and (ii) a streptavidin-binding peptide (SBP), or a nucleic acid encoding this fusion polypeptide.

In the present disclosure, the fusion polypeptide comprising the protein of interest or a functionally or structurally active fragment thereof and a streptavidin-binding peptide (SBP) is term “cargo protein”.

The present invention thus also relates to a cargo protein as described herein.

As disclosed in the EXAMPLES section, within the cargo protein, the streptavidin-binding peptide (SBP) may be fused upstream or downstream of the protein of interest or a functionally or structurally active fragment thereof. Alternatively, within the cargo protein, the streptavidin-binding peptide (SBP) may be fused between the protein of interest and at least one protein, at least one peptide or at least one protein domain.

In one embodiment, the protein of interest is as disclosed herein. In one embodiment, within the cargo protein, the streptavidin-binding peptide (SBP) is fused upstream or downstream of the protein of interest as disclosed herein.

In one embodiment, within the cargo protein, the streptavidin-binding peptide (SBP) is fused between the protein of interest as disclosed herein and at least one protein, such as, for example, a reporter protein. Examples of reporter proteins are disclosed hereinabove.

In one embodiment, within the cargo protein, the streptavidin-binding peptide (SBP) is fused between the protein of interest and at least one peptide or at least one protein domain that can be self-cleaved, as disclosed herein.

An exemplary amino acid sequence of streptavidin-binding peptide is MDEKTTGWRGGHVVEGLAGELEQLRARLEHHPQGQREP (SEQ ID NO: 41) or a fragment thereof, preferably wherein the fragment of streptavidin-binding peptide retains its ability to bind to streptavidin. For instance, such fragment may comprise or consist of the amino acid sequence GGHVVEGLAGELEQLRARLEHHPQGQREP (SEQ ID NO: 42).

Thus, in one embodiment, the streptavidin-binding peptide (SBP) comprises or consists of the sequence with SEQ ID NO: 41 or a fragment thereof. In one embodiment, the streptavidin-binding peptide (SBP) comprises or consists of the sequence with SEQ ID NO: 42.

The skilled artisan is well aware of other suitable streptavidin-binding peptides. For instance, streptavidin-binding peptides have been described in International patent publications WO 2002/38580 or WO 2003/074546.

In some embodiments, the extracellular vesicle-producing cell (or a population of extracellular vesicle-producing cells) is contacted with a nucleic acid encoding the fusion polypeptide “carrier protein”, and with a nucleic acid encoding the fusion polypeptide “cargo protein”. This step may be carried out in particular by transfecting extracellular vesicle-producing cells with the nucleic acids.

In some embodiment, both nucleic acids encoding the fusion polypeptide “carrier protein” and the fusion polypeptide “cargo protein” may be present on a same polynucleotide vector, for instance, on a same plasmid; or alternatively, the two nucleic acids encoding the fusion polypeptide “carrier protein” and the fusion polypeptide “cargo protein” may be on separate polynucleotide vectors, for instance, on separate plasmids.

When the two nucleic acids encoding the fusion polypeptide “carrier protein” and the fusion polypeptide “cargo protein” are on separate polynucleotide vectors, the method may comprise an initial step of contacting an extracellular vesicle-producing cell (or a population of extracellular vesicle-producing cells) with the nucleic acid encoding the fusion polypeptide “carrier protein”; and only afterwards, a step of contacting an extracellular vesicle-producing cell (or a population of extracellular vesicle-producing cells) with the nucleic acid encoding the fusion polypeptide “cargo protein”.

Accordingly, the method may comprise a first step of providing an extracellular vesicle-producing cell (or a population of extracellular vesicle-producing cells) already expressing the fusion polypeptide “carrier protein” (for instance, obtained by the method of targeting a protein of interest in the lumen of an extracellular vesicle described herein); and a second step of contacting this extracellular vesicle-producing cell (or population of extracellular vesicle-producing cells) with the nucleic acid encoding the fusion polypeptide “cargo protein”.

In some embodiments, the targeting of the protein of interest in the lumen of the extracellular vesicle is reversed by addition of biotin or a structural analog thereof. In other words, the protein of interest is released from the fusion polypeptide “carrier protein” by addition of biotin or a structural analog thereof.

As used herein, the term “released” means that the protein of interest is not associated with the fusion polypeptide “carrier protein”. Thus, in one embodiment, after addition of biotin or a structural analog thereof, the protein of interest is free in the lumen of the extracellular vesicle (i.e. not associated with the fusion polypeptide “carrier protein”). As the protein of interest is free in the lumen of the extracellular vesicle, the fusion of said extracellular vesicle to a cell would trigger the release of the protein of interest in the cytoplasm of the cell.

Biotin, also called “vitamin B7”, is well known in the art and possessed the following structure:

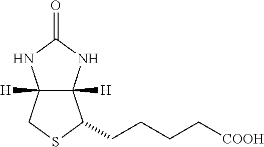 [SMILES: O=C(O)CCCC[C@@H]1SC[C@@H]2NC(=O)N[C@@H]21]

Some examples of biotin analogues include, without limitation, iminobiotin, desthiobiotin, ethylbiotin, biotin carbonate and biotin carbamate.

In the embodiments where the targeting of the protein of interest in the lumen of the extracellular vesicle is reversed by addition of biotin or a structural analog thereof, the streptavidin-binding peptide has a lower affinity for streptavidin than the biotin or a structural analog thereof.

Technics to determine the affinity of a compound are well known to the skilled artisan and include, for example, competition binding assays or surface plasmon resonance.

Thus, in one embodiment, the method comprises the addition of biotin or a structural analog thereof to free the fusion polypeptide “cargo protein” from the fusion polypeptide “carrier protein”.

The method can be performed in vivo, in vitro or ex vivo.

In some embodiments, the extracellular vesicle-producing cell is a HEK293 cell or a cell from a derivative cell line.

In some embodiments, the extracellular vesicle-producing cell is an adipocyte.

In some embodiments, the extracellular vesicle-producing cell is an immune cell, including, but not limited to, a mastocyte, a lymphocyte (such as, e.g., a T-cell or a B-cell), and a dendritic cell.

In some embodiments, the extracellular vesicle-producing cell is a stem cell, including, but not limited to, an embryonic stem cell, an adult stem cell (such as, e.g., a hematopoietic stem cell, a mammary stem cell, an intestinal stem cell, a mesenchymal stem cell, an adipocyte stem cell, an endothelial stem cell, a neural stem cell, an olfactory adult stem cell, or a neural crest stem cell), a cancer stem cell, an induced pluripotent stem cell (iPSC) and an induced stem cell (iSC).

The skilled artisan is aware of methods of obtaining embryonic stem cells, in particular human embryonic stem cells, which methods do not require embryo destruction; see, e.g., Chung et al., 2008. Cell Stem Cell. 2(2):113-117, which is the first of several publications disclosing such a method.

In some embodiments, it may be desirable to recover these extracellular vesicles into the lumen of which a protein of interest has been reversibly targeted. Accordingly, the method may comprise steps of:
contacting an extracellular vesicle-producing cell (or a population of extracellular vesicle-producing cells) with:
   the fusion polypeptide “carrier protein”, or with a nucleic acid encoding this fusion polypeptide, and
   the fusion polypeptide “cargo protein”;
culturing the extracellular vesicle-producing cell (or the population of such cells) in a suitable culture medium; and
recovering the extracellular vesicles produced by the extracellular vesicle-producing cell (or the population of such cells).

General means and methods for culturing the extracellular vesicle-producing cells, and for recovering the extracellular vesicles, are well known in the art. Embodiments described above in relation with the method of targeting a protein of interest in the lumen of an extracellular vesicle apply mutatis mutandis to these aspects of the method.

Thus, the present invention also relates to a method for producing extracellular vesicles comprising, in their lumen, the fusion polypeptide “carrier protein” and the fusion polypeptide “cargo protein” as described herein.

An exemplary protocol of this method is provided in the EXAMPLES section.

The present invention also relates to an extracellular vesicle (or a population of such extracellular vesicles), said extracellular vesicle comprising the fusion polypeptide “carrier protein” and the fusion polypeptide “cargo protein”.

According to the invention, the fusion polypeptide “carrier protein” is located in the lumen of an extracellular vesicle. Still according to the invention, the fusion polypeptide “carrier protein” is anchored or otherwise attached, via its sub-membrane targeting domain, to the inner extracellular vesicle membrane. Still according to this invention, the fusion polypeptide “cargo protein” is non-covalently bound to the fusion polypeptide “carrier protein” in the extracellular vesicle lumen.

In some embodiments, the extracellular vesicle is a small extracellular vesicle.

In some embodiments, the extracellular vesicle is an exosome. Exosomes may have a diameter typically ranging from about 30 nm to about 150 nm, preferably from about 30 nm to about 120 nm, more preferably from about 40 nm to about 80 nm. In particular, exosomes may have a diameter ranging from about 30 nm to about 120 nm.

In some embodiments, the population of extracellular vesicles is monodisperse in aqueous solutions, preferably in a NaCl 0.9% aqueous solution and/or in PBS.

The polypeptides, extracellular vesicles and methods of targeting, either reversibly or not, a protein of interest in the lumen of an extracellular vesicle, can find their use in various applications, in particular in the field of therapy.

Hence, the present invention also relates to the fusion polypeptides described herein, nucleic acids described hereinabove, as well as to extracellular vesicle (or population of such extracellular vesicles) described herein, for use as a drug, and in particular, for use in the prevention and/or treatment of diseases; or else to methods of preventing and/or of treating diseases in a subject in need thereof, comprising administering to the subject the fusion polypeptides described herein, the nucleic acids described hereinabove, or the extracellular vesicle (or population of such extracellular vesicles) described herein.

Diseases that can be prevented and/treated include, without limitation, cancer, genetic lysosomal diseases, diabetes, loss of function diseases, inflammation, infectious diseases, acquired immunodeficiencies, aging, and neurological diseases.

### BRIEF DESCRIPTION OF THE DRAWINGS

FIG. 1 is a schematic representation of Strategy 1. Src: sub-membrane targeting domain; PP: pilot peptide, i.e., a peptide interacting with the ESCRT cellular machinery; EV: extracellular vesicle.

FIG. 2 is a schematic representation of Strategy 2. Scr: sub-membrane targeting domain; PP: pilot peptide, i.e., a peptide interacting with the ESCRT cellular machinery; ZeoR: zeocin resistance protein; PT2A: self-cleavable porcine teschovirus-1 protease 2A; SBP: streptavidin-binding peptide; EV: extracellular vesicle.

FIG. 3 is a Western-blot showing expression of the Src-Nanoluc-PP polypeptide [Src-NLuc-PP] in cell extracts [cells] and in extracellular vesicles [EVs]. The primary antibody targets the pilot peptide PP. The arrow shows the expected molecular weight of the Src-Nanoluc-PP polypeptide.

FIG. 4 is a graph showing a bioluminescence assay carried out on extracellular vesicles comprising the Src-Nanoluc-PP polypeptide [Src-Nluc-PP EV] or not [control EV]. Results are expressed in relative light unit (RLU).

FIG. 5 is a graph showing a bioluminescence assay carried out on extracellular vesicles comprising the Src-Nanoluc-PP polypeptide [EV], and after digestion tests using proteinase K [EV+PK], further in presence of Triton X-100 [EV+Triton+PK], or in presence of Triton X-100 and PMSF [EV+Triton+(PK+PMSF)]. Results are expressed in relative light unit (RLU).

FIG. 6 is a set of 4 Western-blots showing the presence or absence of the Src-Nanoluc-PP polypeptide in the lumen of extracellular vesicles after digestion test. EVs: extracellular vesicles comprising the Src-Nanoluc-PP polypeptide; PP: pilot peptide, i.e., a peptide interacting with the ESCRT cellular machinery. The primary antibodies target the Alix protein, the pilot peptide PP, nanoluciferase [Nanoluc] and CD81, as indicated.

FIG. 7 is a Western-blot showing expression of the Src-streptavidin-PP polypeptide [Src-strepta-PP] in cell extracts [cells] and in extracellular vesicles [EVs]. The primary antibody targets the pilot peptide PP. The arrow shows the expected molecular weight of the Src-streptavidin-PP polypeptide.

FIG. 8 is a set of 4 Western-blots showing the presence or absence of the Src-streptavidin-PP polypeptide in the lumen of extracellular vesicles after digestion test. EVs: extracellular vesicles comprising the Src-streptavidin-PP polypeptide; PP: pilot peptide, i.e., a peptide interacting with the ESCRT cellular machinery. The primary antibodies target the Alix protein, the pilot peptide PP, streptavidin and CD81, as indicated.

FIG. 9 is a set of 2 Western-blots showing expression of the Src-streptavidin-PP polypeptide [Src-Strepta-PP] and of the ZeoR-PT2A-SBP-Nanoluc polypeptide [ZeoR-PT2A-SBP-Nluc] in cell extracts [cells; left panel] and in extracellular vesicles [EVs; right panel]. The primary antibody targets the pilot peptide PP. The arrow shows the expected molecular weight of the Src-streptavidin-PP polypeptide.

FIG. 10 is a set of 2 Western-blots showing expression of the Src-streptavidin-PP polypeptide [Src-Strepta-PP] and of the ZeoR-PT2A-SBP-Nanoluc polypeptide [ZeoR-PT2A-SBP-Nluc] in cell extracts [cells; left panel] and in extracellular vesicles [EVs; right panel]. The primary antibody targets SBP. The upper arrow shows the expected molecular weight of the ZeoR-PT2A-SBP-Nanoluc polypeptide; the lower arrow shows the expected molecular weight of the SBP-Nanoluc polypeptide (upon self-cleavage of PT2A).

FIG. 11 is a set of 2 Western-blots showing expression of the Src-streptavidin-PP polypeptide [Src-Strepta-PP] and of the ZeoR-PT2A-SBP-Nanoluc polypeptide [ZeoR-PT2A-SBP-Nluc] in cell extracts [cells; left panel] and in extracellular vesicles [EVs; right panel]. The primary antibody targets nanoluciferase. The upper arrow shows the expected molecular weight of the ZeoR-PT2A-SBP-Nanoluc polypeptide; the lower arrow shows the expected molecular weight of the SBP-Nanoluc polypeptide (upon self-cleavage of PT2A).

FIG. 12 is a graph showing a bioluminescence assay carried out on extracellular vesicles comprising both the Src-streptavidin-PP polypeptide and ZeoR-PT2A-SBP-Nanoluc polypeptide [EV], and after digestion tests using proteinase K [EV+PK], further in presence of Triton [EV+Triton+PK], or in presence of Triton and PMSF [EV+Triton+(PK+PMSF)]. Results are expressed in relative light unit (RLU).

FIG. 13 is a set of 4 Western-blots showing the presence or absence of the Src-streptavidin-PP polypeptide and ZeoR-PT2A-SBP-Nanoluc polypeptide in the lumen of extracellular vesicles after digestion test. EVs: extracellular vesicles comprising both the Src-streptavidin-PP polypeptide and ZeoR-PT2A-SBP-Nanoluc polypeptide; PP: pilot peptide, i.e., a peptide interacting with the ESCRT cellular machinery. The primary antibodies target the Alix protein, the pilot peptide PP, nanoluciferase [Nanoluc] and CD81, as indicated.

FIG. 14 is a set of 4 Western-blots showing the presence or absence of the SBP-Nanoluc polypeptide associated with the Src-streptavidin-PP polypeptide in the lumen of extracellular vesicles, in presence of biotin [+Biotin] or in absence of biotin [Ø Biotin], in cell extracts [cells] and in extracellular vesicles [EVs]. The primary antibodies target the Alix protein, the pilot peptide PP and nanoluciferase [Nanoluc], as indicated. The upper arrow for nanoluciferase shows the expected molecular weight of the ZeoR-PT2A-SBP-Nanoluc polypeptide; the lower arrow shows the expected molecular weight of the SBP-Nanoluc polypeptide (upon self-cleavage of PT2A).

FIG. 15 is a graph showing a bioluminescence assay carried out on empty extracellular vesicles [Control] or on extracellular vesicles produced in cells expressing only the ZeoR-PT2A-SBP-Nanoluc polypeptide [SBP-NLuc], or on extracellular vesicles produced in cells expressing both the Src-streptavidin-PP polypeptide and the ZeoR-PT2A-SBP-Nanoluc polypeptide in absence [CP+SBP-NLuc wo Biotine] or in presence [CP+SBP-NLuc+1 mM Biotine] of biotin. Results are expressed in relative light unit (RLU).

FIGS. 16A-C are a set of 3 Western-blots showing the presence or absence of a (ZeoR-PT2A-)SBP-Oct4 polypeptide associated with the Src-streptavidin-PP polypeptide [Src-strepta-PP] in the lumen of extracellular vesicles [EVs]. The primary antibody targets SBP (FIG. 16A), Oct4 (FIG. 16B) or the pilot peptide PP (FIG. 16C). On FIGS. 16A-B, the upper arrow shows the expected molecular weight of the ZeoR-PT2A-SBP-Oct4 polypeptide; the lower arrow shows the expected molecular weight of the SBP-Oct4 polypeptide (upon self-cleavage of PT2A). On FIG. 16C, the arrow shows the expected molecular weight of the Src-streptavidin-PP polypeptide.

FIGS. 17A-C are a set of 3 Western-blots showing the presence or absence of a (ZeoR-PT2A-)SBP-eIF4G polypeptide associated with the Src-streptavidin-PP polypeptide [Src-strepta-PP] in the lumen of extracellular vesicles [EVs]. The primary antibody targets SBP (FIG. 17A), eIF4G (FIG. 17B) or the pilot peptide PP (FIG. 17C). On FIGS. 17A-B, the upper arrow shows the expected molecular weight of the ZeoR-PT2A-SBP-eIF4G polypeptide; the lower arrow shows the expected molecular weight of the SBP-eIF4G polypeptide (upon self-cleavage of PT2A). On FIG. 17C, the arrow shows the expected molecular weight of the Src-streptavidin-PP polypeptide.

FIG. 18 is a set of 3 Western blots showing the presence of asparaginase-SBP polypeptide, when associated with the Src streptavidin PP polypeptide [Src-Strepta-PP], in the lumen of extracellular vesicles [EVs]. The primary antibodies target SBP peptide.

FIG. 19 is a set of 3 Western blots showing expression of the SBP-Oct4-eGFP polypeptide and SBP-eGFP polypeptide, associated with a Src-streptavidin-PP polypeptide [Src-Strepta-PP] in extracellular vesicles [EVs]. The primary antibodies target SBP peptide.

FIG. 20 is a set of 3 Western blots showing the presence of Oct4-SBP-eGFP polypeptide, when associated with the Src streptavidin PP polypeptide [Src-Strepta-PP], in the lumen of extracellular vesicles [EVs]. The primary antibodies target SBP peptide.

### EXAMPLES

The present invention is further illustrated by the following examples.

### Example 1

### Materials and Methods

### Expression Vectors and Molecular Cloning

The expression system to sort proteins at the inward membrane of extracellular vesicles (EVs) is based on a Ciloa SAS's patent (patents EP 2 480 672 B1 and U.S. Pat. No. 9,611,481 B2). In details, a Src peptide comprising a myristic acid on its N-terminus glycine residue allows the anchoring of a protein of interest to which it is fused in the EV membrane. Another peptide, referred to as pilot peptide (PP), allows the sorting of proteins of interest to which it is fused inside the EVs. Therefore, fusing the coding sequence of a protein of interest in-between a Src peptide and a PP allows to address the protein of interest at the inward membrane of EVs.

Genes encoding streptavidin, nanoluciferase (Nanoluc), eIF4G, Oct4, OMOMyc, asparaginase II, eGFP, and Oct4 fused to eGFP, were codon-optimized for expression in human cells and cloned into in-house eukaryotic expression plasmid vectors.

Molecular Cloning in Expression Vectors for Cargo Loading into EVs

The nucleic acids encoding the proteins of interest (herein also referred to as “cargo proteins”) streptavidin, Nanoluc and asparaginase II were fused downstream of a Src peptide and upstream of a PP to generate a Src-protein-PP polyprotein sorted at the inward membrane of EVs.

Molecular Cloning in Expression Vectors for Reversible Cargo Loading into EVs

In order to load proteins into the EVs in a reversible manner, genes encoding the cargo proteins Nanoluc, eIF4G, Oct4, OMOMyc and asparaginase II were fused either i) downstream of ZeoR (zeocine resistance), self-cleavable PT2A (porcine teschovirus-1 2A) peptide and streptavidin-binding peptide (SBP) sequences to generate an autocleavable (ZeoR-PT2A)-SBP-cargo polyprotein; or ii) downstream of a SBP sequence to generate an SBP-cargo polyprotein; or iii) upstream of a SBP sequence, to generate a cargo-SBP polyprotein.

### Production by Mammalian Cells of EVs Containing Cargo Proteins

HEK293T cells were cultured in DMEM supplemented with 5% of heat-inactivated fetal bovine serum (iFBS), 2 mM of GlutaMAX and 5 μg/mL of gentamicin at 37° C. in a 5% CO₂ humidified incubator.

HEK293T cells were plated into flasks or cell chambers in complete medium and were transfected with DNA expression plasmids using PEI.

For the reversible loading of a cargo protein, a nucleic acid encoding Src-streptavidin-PP was co-transfected with the DNA expression plasmid encoding a cargo protein fused to SBP (either Zeo-PT2A-SBP-cargo or SBP-cargo or cargo-SBP or cargo-SBP-cargo or SBP-cargo1-cargo2). SBP interacts with streptavidin, allowing the loading of SBP-cargo into the lumen of EVs.

Twenty-four hours post-transfection, cultures were fed with a serum-free culture medium and incubated for a further 48 hours.

To study the effect of biotin on the reversible loading of the cargo protein, 1 mM of Biotin (Analytic Lab, ref. B4639-100MG) was added to the serum-free culture medium twenty-four hours post-transfection.

### EV Purification

Cell culture medium was harvested from transiently transfected HEK293T cells and EV isolation was performed. Briefly, cell culture supernatant was clarified by two consecutive centrifugations (1 300 rpm for 10 minutes then 4 000 rpm for 15 minutes), followed by filtration through a 0.22-μm membrane filter. The supernatant was then ultrafiltered and diafiltered on membranes (30 kDa or 300 kDa) and purified by multimodal ion-exchange chromatography using NGC system (Bio-Rad). Fractions containing EV-associated cargo proteins were identified by Western-blotting.

### Ultracentrifugation

Clarified cell culture medium or pure EV preparations were used, and ultracentrifugation was performed in the Optima MAX-XP instrument. Samples were centrifuged for 25 minutes at 120 000 g in a MLA-130 rotor (Beckman Coulter) in 1-mL open-top thickwall polycarbonate tubes (#343778), or for 33 minutes in a TLA-100.3 rotor (Beckman Coulter) in 3.5-mL open-top thickwall polycarbonate tubes (#349622).

After removing the supernatant, EV pellets were resuspended either in TNE 1× for subsequent proteinase K digestions, or in Laemmli buffer and denatured for 5 minutes at 95° C. for Western-blotting.

Protection from Proteinase K Digestion

In order to determine the localization of the cargo proteins, protection from proteinase K (PK) digestion was evaluated. EVs were incubated for 1 hour at 37° C. with 0.05 mg/mL of PK (Fisher BioReagents™, ref. 10172903) in TNE 1×. For permeabilized EVs, 1% Triton X-100 (Sigma, ref. 93443-100ML, 10%) was added for 10 minutes at 4° C. PK was then inactivated by adding 5 mM of phenylmethylsulfonyl fluoride (PMSF) (Fisher BioReagents™, ref. 10485015) for 5 minutes at 37° C. For control of efficiency of PMSF, the PMSF was added to PK before incubation with EVs.

The presence and/or the activity of the cargo proteins after PK treatment was evaluated by Western-blotting and/or luminescence assay.

### Luminescence Assay

The luminescence assay was carried out on clarified cell culture medium or on purified EVs.

Nanoluc luminescence was revealed using the Nano-Glo© Luciferase assay (Promega, ref. N1120). Briefly, 50 μL of EVs were incubated with 50 μL of the substrate buffer mixture provided in the kit (1:50), for 3 min at room temperature, with shaking at 300 rpm and protected from light. Luminescence was measured using a CLARIOstar Plus plate reader (BMG Labtech) at 470-480 nm.

### TCA Precipitation

EVs were precipitated using 20% trichloroacetic acid (TCA) for concentration before Western-blotting. EVs were incubated in 20% TCA for 30 minutes at 4° C., then centrifuged at 14 000 rpm for 10 minutes at 4° C. The supernatant was discarded and another centrifugation at 14 000 rpm for 5 minutes at 4° C. was carried out. Any remaining supernatant was discarded and the pellet was resuspended in denaturation Laemmli buffer and heated for 5 min at 95° C.

### SDS-PAGE, Western-Blotting (WB) and Antibodies

Protein concentration of cellular extracts was measured using the BCA assay (Pierce BCA Protein Assay kit, ThermoFisher Scientific). EVs and cell extracts preparations were separated by SDS-PAGE on a 4-15% acrylamide gel (4-15% Mini-PROTEAN® TGX Stain-Free™ Gel, Bio-Rad) and subsequently transferred onto a PVDF membrane.

The immunodetection of cargo proteins was performed with primary antibodies against PP (in-house antibody raised in rabbit, Proteogenix), SBP (Santa Cruz, ref. sc-101595 or immunopurified polyclonal rabbit anti-SBP—AB011823-1, Proteogenix), Nanoluc (mouse monoclonal antibody, Promega, ref. N700A), Oct4 (rabbit monoclonal antibody, Arigo Biolaboratories, ref. ARG66826), eTF4G (rabbit monoclonal antibody, Invitrogen, ref. MA5-14914), Alix (rabbit polyclonal antibody, Proteintech, ref. 12422-1-AP), CD81 (rabbit polyclonal antibody, Genetex, ref. GTX101766) and asparaginase II (rabbit anti-L-asparaginase II antibody, Rockland, ref. 100-4171).

Membranes were then incubated with the corresponding secondary HRP-conjugated antibodies (donkey anti-mouse or anti-rabbit HRP, Jackson ImmunoResearch, ref. 715-035-150 or 711-035-152). The signal was detected using an enhanced chemiluminescence detection kit (Super Signal West Pico Plus, ThermoFischer Scientific, ref. 34580; or Clarity Max Western ECL Substrate, Bio-Rad, ref. 1705062) and membranes imaged with ChemiDoc Imaging System (Bio-Rad).

### Results

### Technological Concept

### Targeting of a Protein of Interest in EVs

On the one hand, a Src domain comprising a myristic acid on its N-terminal glycine residue allows the anchorage of proteins on the outer membrane surface of EVs; on the other hand, a “pilot peptide” (PP) that interacts with ESCRT proteins ensures the delivery of proteins inside EVs, in particular inside exosomes.

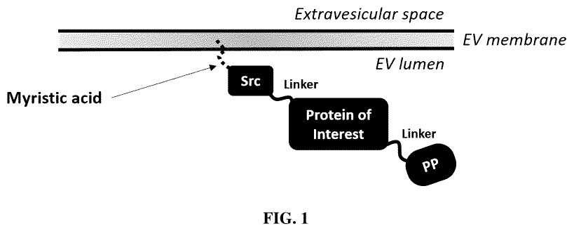

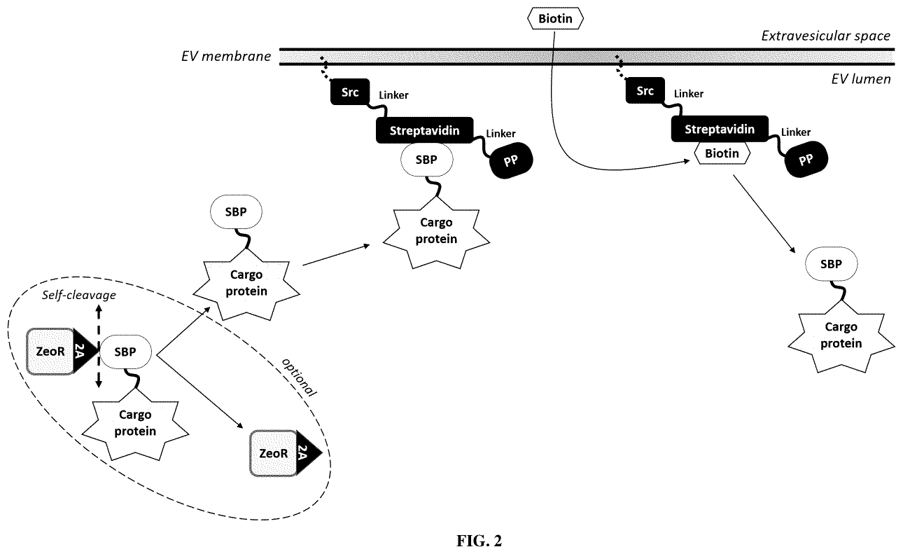

The underlying premise was that a protein of interest, when its sequence is inserted in-between a Src domain and a pilot peptide, would be targeted inside the EVs and ultimately be anchored in their inner membrane (FIG. 1).

Here, the proteins of interest targeted to the inner membrane of EVs are Nanoluc, streptavidin and asparaginase II.

Nanoluc makes it possible to have bioluminescent EVs, making it possible to monitor them, in particular in vivo. Streptavidin is known to strongly interact with biotin but also with peptides such as streptavidin-binding peptide (SBP). Escherichia coli asparaginase II is an enzyme that hydrolyzes asparagine, with applications in anti-cancer therapies.

### Reversible Loading of a Protein of Interest in EVs

We have developed a technology making it possible to reversibly address any type of protein in the lumen of EVs by using a chimeric Src-streptavidin-PP polypeptide serving as carrier. A streptavidin-binding peptide (SBP) is then used for a reversible binding with streptavidin. The underlying premise was that addition of biotin should, by competition, free SBP from streptavidin.

Hence, a fusion polypeptide comprising SBP and a protein of interest would address and retain this protein of interest inside the EVs. Addition of biotin would then compete with SBP for binding to streptavidin, releasing the protein of interest inside the EVs, or inside a target cell after EV uptake (FIG. 2).

Here, the proteins of interest reversibly targeted to the inner membrane of EVs are Nanoluc, streptavidin, asparaginase II, and OMOMyc.

Nanoluc makes it possible to have bioluminescent EVs, making it possible to monitor them, in particular in vivo. Streptavidin is known to strongly interact with biotin but also with peptides such as streptavidin-binding peptide (SBP). Escherichia coli asparaginase II is an enzyme that hydrolyzes asparagine, with applications in anti-cancer therapies. OMOMyc (transdominant negative Myc mutant) also offers prospects in anti-cancer therapy because it allows tumor cells to enter apoptosis. Oct4 (nuclear transcription factor) is targeted to the nucleus thanks to its NLS (nuclear localization Signal) and play a role in cell fate. eGFP (enhanced Green Fluorescent Protein) offers the possibility to trace a protein with which it is merged by looking under fluorescent microscope.

Our objectives were thus to demonstrate the expression of proteins of interest and their addressing inside EVs, either by direct interaction and stable with the inner membrane (strategy 1), or by reversible interaction with a protein targeted and anchored in the inner membrane of EVs (strategy 2). The reversibility of the interaction between streptavidin and SBP was also be studied.

### Strategy 1: Targeting of a Protein of Interest in EVs

Several proteins of interest (or cargo proteins) were addressed to the inner membrane of EVs using a Src peptide and a pilot peptide (PP):
NanoLuc, in a Src-Nanoluc-PP construct;
streptavidin, in a Src-streptavidin-PP construct (also referred to as “carrier” in the following); and
asparaginase II, in a Src-asparaginase II-PP construct.

The objective was to demonstrate expression of these proteins of interest in cells, and their association with, and localization inside, EVs.

### Src-Nanoluc-PP

The Src-Nanoluc-PP polypeptide allows to obtain bioluminescent EVs, easily traceable in vitro and in vivo.

### Expression in Cells and EV Targeting

As seen on FIG. 3, the Src-Nanoluc-PP polyprotein was expressed in cells and was found to be associated with EVs.

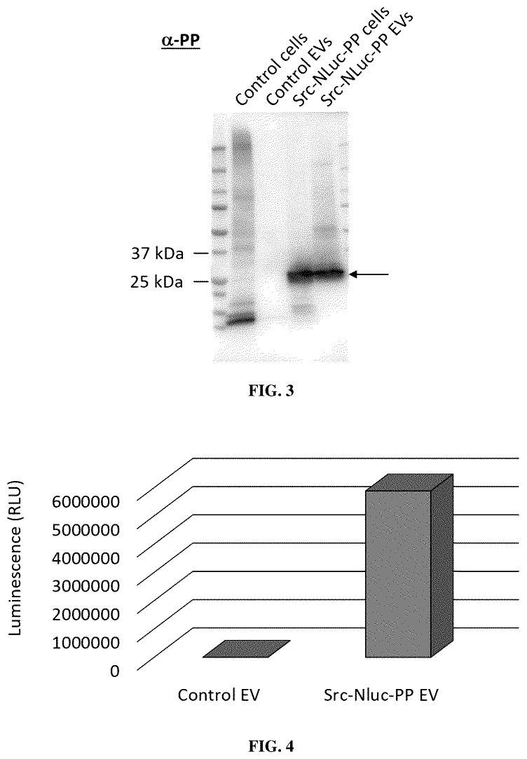

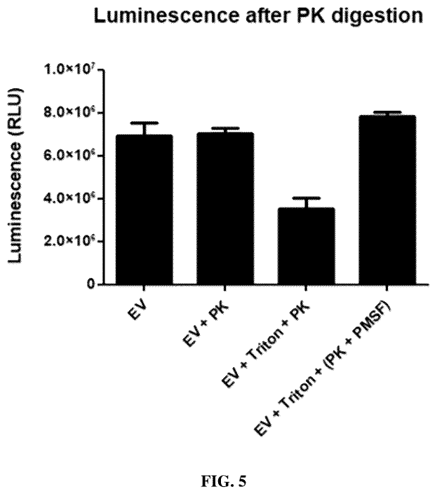

As seen on FIG. 4, there was luminescence associated with the Src-Nanoluc-PP EVs, confirming the association of Nanoluc with EVs.

According to these results, the Src-Nanoluc-PP polypeptide was functional and associated with EVs. The objective was then to demonstrate that this polypeptide was localized inside the EVs.

### Localisation in EVs

In order to determine the localization of the Src-Nanoluc-PP polypeptide, a digestion test with a protease (proteinase K) was carried out. The proteins located inside the EVs are protected by the vesicle membrane and would therefore not be hydrolyzed by proteinase K. In the presence of Triton X-100, a detergent that permeabilizes the EV membrane, internal and external proteins are accessible to proteinase K and can be hydrolyzed. The accessibility or not of the Src-Nanoluc-PP protein was monitored by luminescence and by SDS-PAGE and Western-blotting.

The following conditions were tested:
Untreated EVs: all proteins should be visible;
EVs+PK: external protein should be degraded, hence not visible anymore;
EVs+Triton+PK: Triton permeabilizes EV membranes, hence all proteins, whether internal or external, should be degraded and not visible anymore;
EVs+Triton+(PK+PMSF): PMSF inactivates PK, hence no protein should be degraded (used as a control of PK specificity).
   Luminescence Analysis after Digestion Tests

As seen on FIG. 5, the only significant difference was observed in the EVs+Triton+PK condition. The EV+Triton condition showed a luminescence similar to the control condition without treatment, hence confirming that the Src-Nanoluc-PP polypeptide was not located outside the EVs.

Western-Blotting Analysis after Digestion Tests

By Western-blotting, the same treatment conditions as those presented above were performed on 3 μg of Src-Nanoluc-PP EVs. The Alix and CD81 proteins (EV markers located respectively inside and outside EVs) were monitored as controls for proteinase K accessibility. The Src-Nanoluc-PP polypeptide was revealed with anti-PP and anti-Nanoluc antibodies.

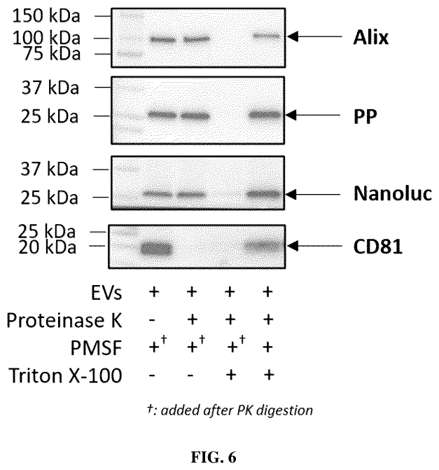

As seen on FIG. 6, the results obtained with Alix are typical of a protein located inside the EVs (digested only in the presence of Triton); conversely, the results obtained with CD81 are typical of a protein located outside the EVs (digested in the presence of PK, with or without Triton).

The results obtained with the Src-Nanoluc-PP polypeptide were identical to that of Alix, confirming that the Src-Nanoluc-PP polypeptide is located inside EVs.

### Src-Streptavidin-PP

The Src-streptavidin-PP polypeptide is also referred herein to as carrier protein. This polypeptide is of interest for reversible loading of a protein of interest (see Strategy 2).

### Expression in Cells and EV Targeting

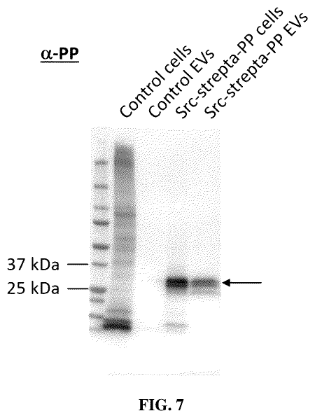

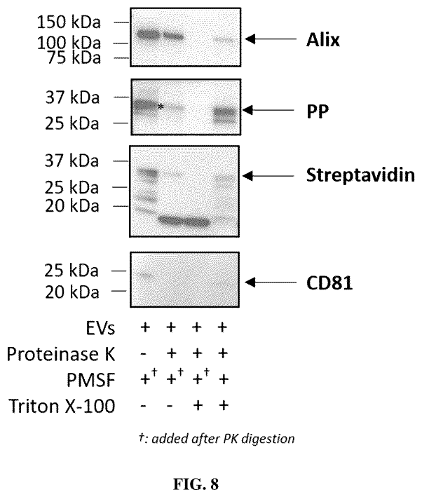

As seen on FIG. 7, the Src-streptavidin-PP polypeptide was expressed in cells and was found to be associated with EVs.

### Localisation in EVs

In order to determine the localization of the Src-streptavidin-PP polypeptide, a digestion test with a protease (proteinase K) was carried out. The proteins located inside the EVs are protected by the vesicle membrane and would therefore not be hydrolyzed by proteinase K. In the presence of Triton X-100, a detergent that permeabilizes the EV membrane, internal and external proteins are accessible to proteinase K and can be hydrolyzed. The accessibility or not of the Src-streptavidin-PP polypeptide was monitored by SDS-PAGE and Western-blotting. The Alix and CD81 proteins (EV markers located respectively inside and outside EVs) were monitored as controls for proteinase K accessibility. The Src-streptavidin-PP polypeptide was revealed with anti-PP and anti-streptavidin antibodies.

The following conditions were tested:
Untreated EVs: all proteins should be visible;
EVs+PK: external protein should be degraded, hence not visible anymore;
EVs+Triton+PK: Triton permeabilizes EV membranes, hence all proteins, whether internal or external, should be degraded and not visible anymore;
EVs+Triton+(PK+PMSF): PMSF inactivates PK, hence no protein should be degraded (used as a control of PK specificity).

As seen on FIG. 8, the results obtained with Alix are typical of a protein located inside the EVs (digested only in the presence of Triton); conversely, the results obtained with CD81 are typical of a protein located outside the EVs (digested in the presence of PK, with or without Triton).

The results obtained with the Src-streptavidin-PP polypeptide were similar to that of Alix, confirming that the Src-streptavidin-PP polypeptide is located inside EVs.

We noted that part of the Src-streptavidin-PP polypeptide was digested in absence of Triton, indicating that a fraction of the Src-streptavidin-PP polypeptide could be located outside EVs; however, a decent fraction was still confirmed to be located inside, confirming the relevance of seeking reversible loading of proteins of interest inside EVs via an interaction between the Src-streptavidin-PP polypeptide and a cargo-SBP fusion (see Strategy 2).

### Strategy 2: Reversible Loading of a Protein of Interest in EVs

Another objective was to load a protein of interest (also referred to as “cargo” in the following) into the EVs, which could be released “on demand” in a target cell, tissue or organ, to exert its action. The method that we have developed is based on an interaction between streptavidin and streptavidin-binding peptide (SBP). This interaction can be undone in the presence of biotin.

As a prerequisite, we have shown above that a carrier protein (the Src-streptavidin-PP polypeptide) could be expressed and localized in EVs.

### SBP-Nanoluc Cargo

To establish proof-of-concept, we aimed at demonstrating that a SBP-Nanoluc cargo could be addressed inside EVs through its interaction with streptavidin, itself located inside EVs.

### Expression in Cells and EV Targeting

Cellular expression and addressing of the cargo in EVs was monitored by SDS-PAGE and Western-blotting. EVs produced by cells co-transfected with (i) the carrier protein, i.e., Src-streptavidin-PP and (ii) the cargo protein, here, (ZeoR-PT2A-)SBP-Nanoluc makes it possible to assess if the presence of both allows the loading of the Nanoluc cargo inside EVs by interaction of SBP with streptavidin.

We used Nanoluc as cargo for ease of traceability in vitro and in vivo; however, it is apparent to the skilled artisan that any other protein of interest can be used as cargo.

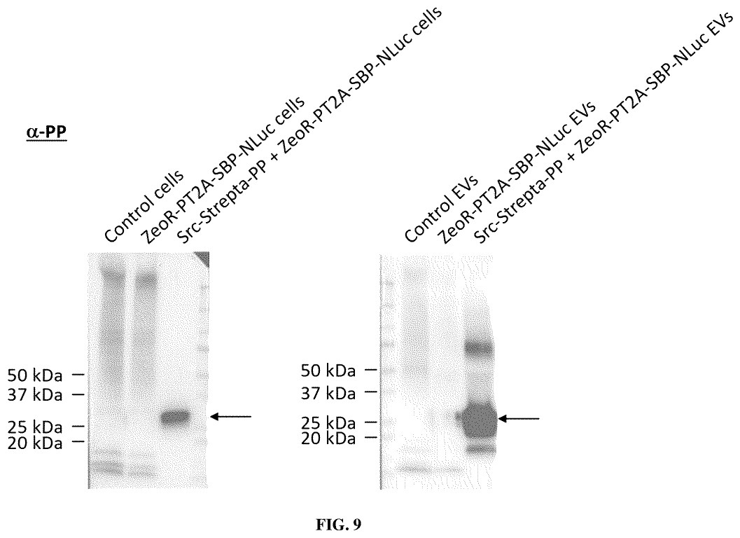

As seen on FIG. 9, the Src-streptavidin-PP “carrier” polyprotein was expressed in cells and was found to be associated with EVs.

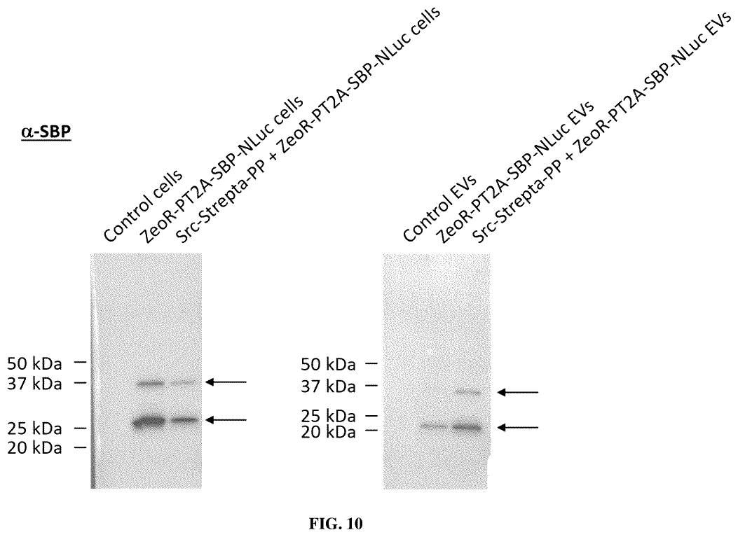

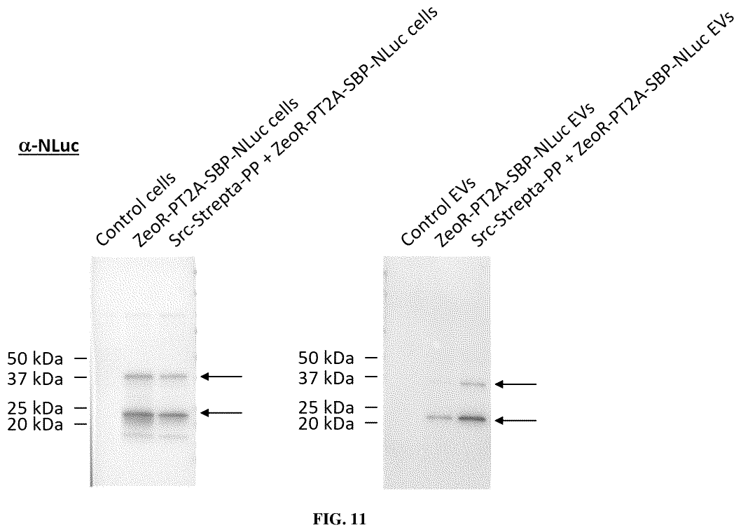

As seen on FIGS. 10 & 11, the (ZeoR-PT2A-)SBP-Nanoluc cargo protein was well expressed in the cells, and much more intense in EVs when co-expressed with the Src-streptavidin-PP “carrier” polyprotein, demonstrating that the streptavidin carrier is capable of efficiently addressing the cargo protein inside EVs.

### Localisation in EVs

Localization of the reversible SBP-Nanoluc cargo associated with EVs was monitored by luminescence and by SDS-PAGE and Western-blotting, using the same proteinase K digestion tests as described above.

### Luminescence Analysis

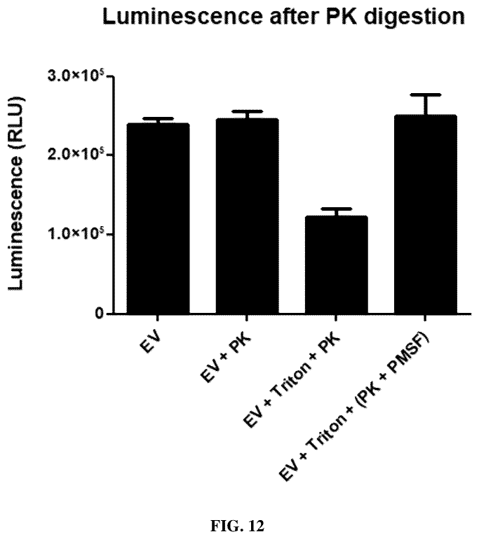

As seen on FIG. 12, the only significant difference was observed in the EVs+Triton+PK condition. The EV+Triton condition showed a luminescence similar to the control condition without treatment, hence confirming that the SBP-Nanoluc cargo protein was not located outside the EVs.

### Western-Blotting Analysis

By Western-blotting, we aimed at localizing the carrier protein (Src-streptavidin-PP polypeptide) and the cargo protein (SBP-Nanoluc). The Alix and CD81 proteins (EV markers located respectively inside and outside EVs) were monitored as controls for proteinase K accessibility. The carrier protein was revealed with anti-PP antibodies, and the cargo protein was revealed with anti-Nanoluc antibodies.

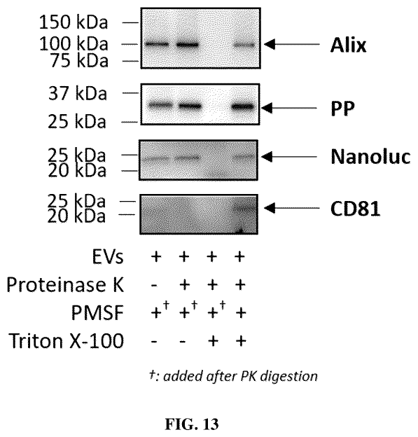

As seen on FIG. 13, the results obtained with Alix are typical of a protein located inside the EVs (digested only in the presence of Triton); conversely, the results obtained with CD81 are typical of a protein located outside the EVs (digested in the presence of PK, with or without Triton).

The results obtained with anti-PP and anti-Nanoluc antibodies were identical to that of Alix, confirming that both the carrier protein and the cargo protein are located inside EVs.

Hence, the cargo protein could indeed be successfully driven into the EV lumen through the interaction of its SBP moiety with the streptavidin moiety of the carrier protein.

This non-covalent interaction is potentially reversible in the presence of biotin.

### Reversibility of Cargo Loading by Addition of Biotin

Src-streptavidin-PP/(ZeoR-PT2A-)SBP-Nanoluc EVs were produced. 1 mM of biotin was added during EV production. An EV production without addition of biotin was performed as negative control.

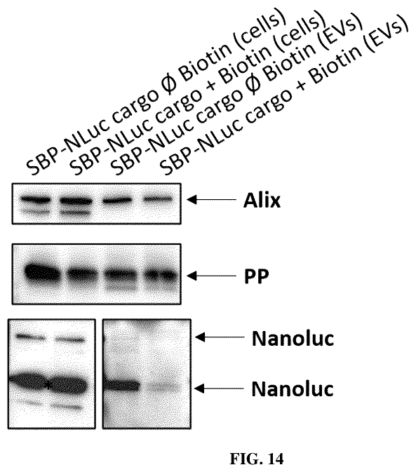

As seen on FIG. 14, in the presence of biotin, the cargo protein is only weakly detected in EVs, by comparison to the negative control. Relative quantification by normalization of the Alix band intensity confirms that the Nanoluc signal is about 2.25 times weaker in presence of biotin by comparison to the negative control.

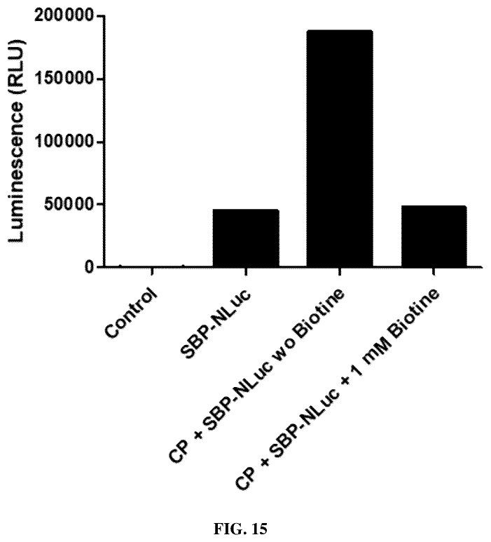

These results were confirmed by a luminescence test. As shown on FIG. 15, in the presence of biotin, luminescence is comparable to that of EVs that are not loaded with the Nanoluc cargo (in absence of carrier protein).

Hence, these results confirm that the loading of the (ZeoR-PT2A-)SBP-Nanoluc cargo protein into EVs is mediated by the streptavidin-SBP interaction; and that this interaction can be reversed in the presence of biotin.

### Conclusion et Perspectives

The results that we have obtained confirm that addressing a protein of interest to the inner membrane of EVs is possible by fusing it to a Src peptide and a pilot peptide interacting with ESCRT proteins. In this proof-of-concept experiment, Nanoluc and streptavidin were successfully addressed to the inner membrane of EVs (Strategy 1).

We have further demonstrated that streptavidin, targeted and anchored in the inner EV membrane, can serve as a carrier protein to target other proteins of interest in the lumen of EVs, when these proteins of interest are fused to the SBP. This proof-of-concept was obtained with an SBP-Nanoluc cargo.

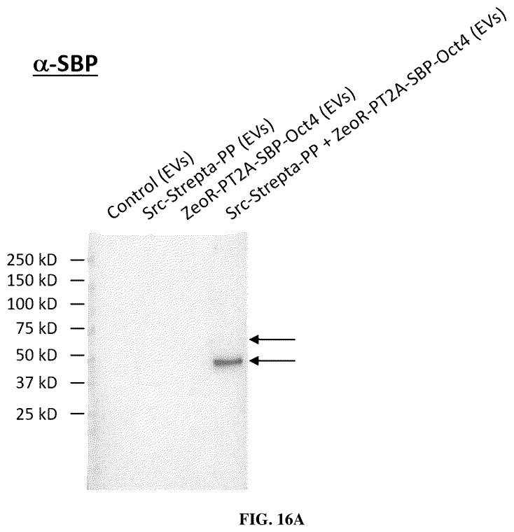

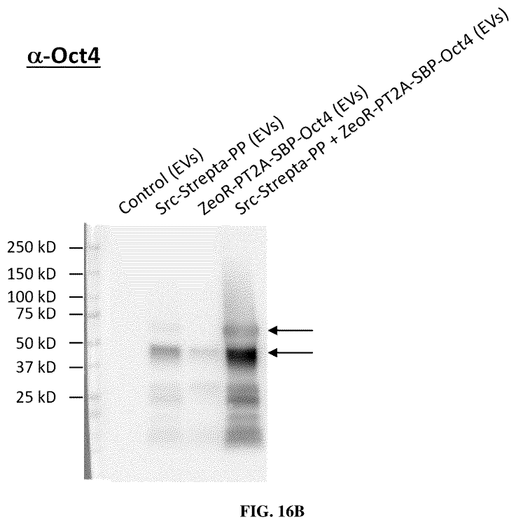

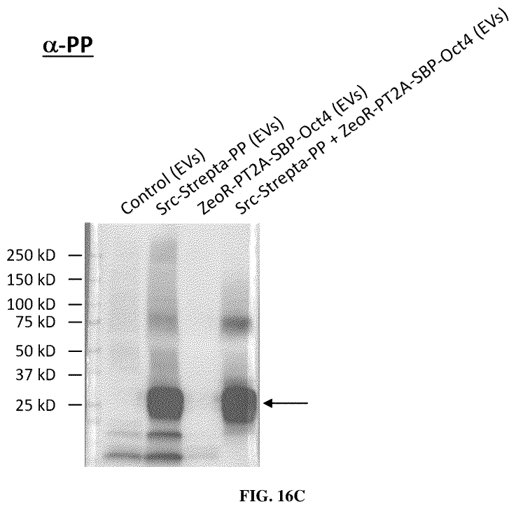

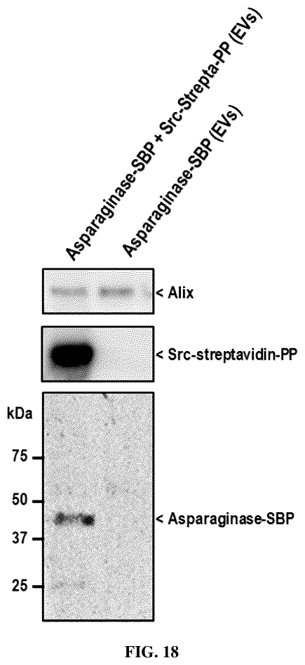

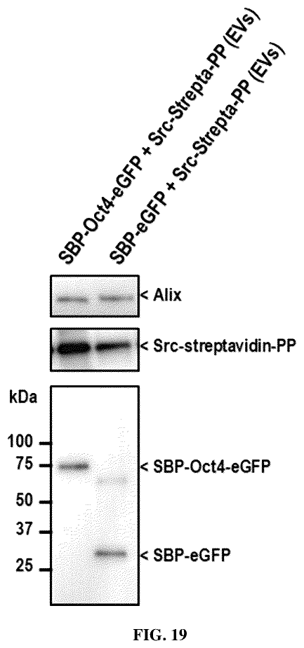

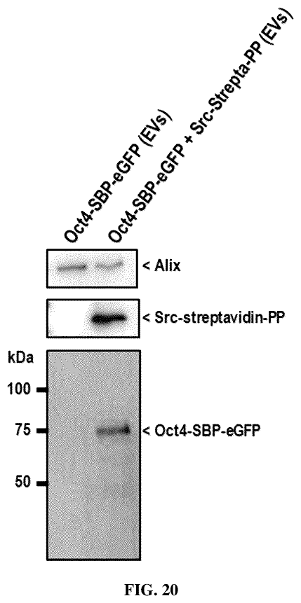

We obtained similar results with other proteins of interest, Oct-4 (FIG. 16), Asparaginase II (FIG. 18), eGFP (SBP-eGFP, FIG. 19 right lane), and Oct4 fused to eGFP with the SBP peptide at two different positions (SBP-Oct4-eGFP, FIG. 19 left lane and Oct4-SBP-eGFP, FIG. 20).

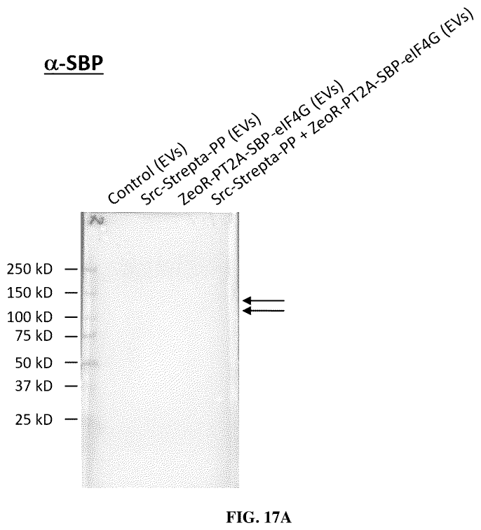

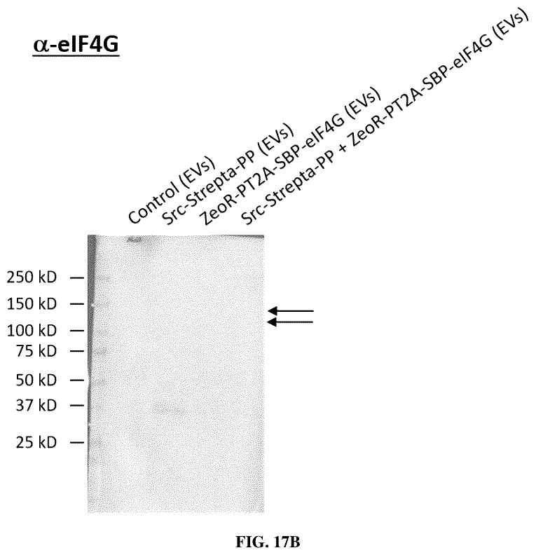

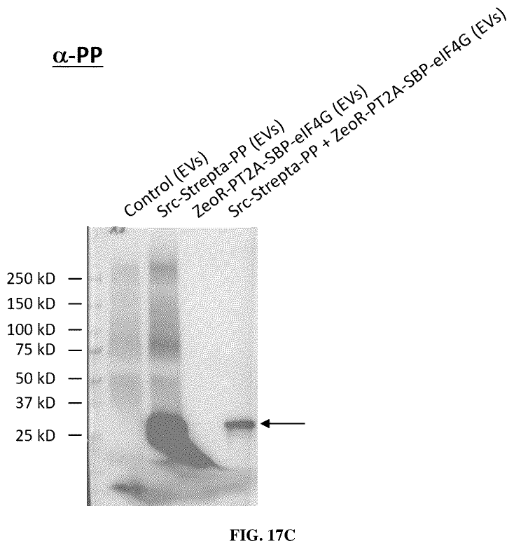

Results with still another protein, eIF4G, did however not show an efficient targeting inside EVs (FIG. 17). We hypothesized that eIF4G being a ribosomal protein, it would be recruited by the neighboring ribosomes directly during/after translation, without ensuring sufficient time to access the EVs and interact with the carrier protein.

The above results indicate that non-ribosomal proteins fused to the SBP polypeptide, like nuclear transcription factor (e.g. Oct4), a bacterial enzyme (e.g. Asparaginase II), and a fluorescent protein (e.g. eGFP), are efficiently targeted to EVs thanks to the interaction of SBP with the streptavidin moiety of Src-streptavidin-PP polypeptide.

Finally, we were able to demonstrate that the loading of a protein of interest inside EVs through a streptavidin-SBP interaction could be efficiently inhibited or reserved by the addition of biotin.

### Sequences

The following sequences were used.

### Cargo Proteins Anchored at the Inward Membrane of Extracellular Vesicles (EVs) by a Myristoylated Src Protein

The following sequences correspond to Src-cargo-PP polypeptides described in the EXAMPLES section.

### Src-Streptavidin-PP

Nucleotide sequence (SEQ ID NO: 43)

| | |
|---|---|
| Src | ATGGGCAGCAGCAAGAGCAAGCCCAAGGATCCCAGCCA |
| | GCGGCGGAGA |
| Linker | AAAAGTAGAGGACCTGGCGGTGGATCTGGCGGAGGAAG |
| | CGGTGGCGGTTCAGGCGGAGGATCTACCGGTATG |
| Streptavidin | GACCCCAGCAAGGACAGCAAGGCCCAAGTGTCTGCTGC |
| | CGAGGCTGGAATCACAGGCACCTGGTATAATCAGCTGG |
| | GCAGCACCTTCATCGTGACCGCTGGTGCTGATGGCGCTC |
| | TGACAGGCACATATGAGAGCGCCGTGGGCAATGCCGAG |
| | AGCAGATATGTGCTGACCGGCAGATACGATAGCGCCCCT |
| | GCCACAGATGGAAGCGGAACAGCTCTTGGATGGACCGT |
| | GGCCTGGAAGAACAACTACAGAAACGCCCACAGCGCCA |
| | CCACTTGGAGCGGCCAATATGTTGGCGGAGCCGAGGCC |
| | AGAATCAACACCCAATGGCTGCTGACCAGCGGCACCAC |
| | AGAGGCCAATGCCTGGAAGTCTACACTCGTGGGCCACG |
| | ACACCTTCACCAAAGTGAAACCTAGCGCCGCCTCCATCG |
| | ACGCCGCTAAAAAAGCCGGCGTGAACAACGGCAACCCT |
| | CTGGATGCTGTTCAGCAA |
| Linker | GGTGGTGGTAGCGGAGGTGGAAGTGGCGGAGGCAGTGG |
| | CGGAGGCTCTAGAGGC |
| Pilot peptide | GCGCCCCACTTCCCTGAAATCTCCTTCCCCCCTAAACCC |
| | GATTCTGATTATCAGGCCTTGCTACCATCCGCGCCAGAG |
| | ATCTACTCTCACCTCTCCCCCACCAAACCCGATTACATC |
| | AACCTTCGACCGGCGCCCTAA |

Protein sequence (SEQ ID NO: 44)

| | |
|---|---|
| Src | MGSSKSKPKDPSQRRR |
| Linker | KSRGPGGGSGGGSGGGSGGGSTGM |
| Streptavidin | DPSKDSKAQVSAAEAGITGTWYNQLGSTFIVTAGADGALT |
| | GTYESAVGNAESRYVLTGRYDSAPATDGSGTALGWTVAW |
| | KNNYRNAHSATTWSGQYVGGAEARINTQWLLTSGTTEAN |
| | AWKSTLVGHDTFTKVKPSAASIDAAKKAGVNNGNPLDAV |
| | QQ |
| Linker | GGGSGGGSGGGSGGGSRG |
| Pilot peptide | APHFPEISFPPKPDSDYQALLPSAPEIYSHLSPTKPDYINLRP |
| | AP |

### Src-Nanoluc-PP

Nucleotide sequence (SEQ ID NO: 45)

| | |
|---|---|
| Src | ATGGGCAGCAGCAAGAGCAAGCCCAAGGATCCCAGCCA |
| | GCGGCGGAGA |
| Linker | AAAAGTAGAGGACCTGGCGGTGGATCTGGCGGAGGAAG |
| | CGGTGGCGGTTCAGGCGGAGGATCTACCGGT |
| NanoLuc | ATGGTGTTCACCCTGGAAGATTTCGTCGGCGACTGGCGG |
| | CAGACAGCCGGCTATAATCTGGACCAGGTGCTGGAACA |
| | AGGCGGCGTGTCCAGCCTGTTTCAGAACCTGGGAGTGTC |
| | CGTGACACCCATCCAGAGAATCGTGCTGAGCGGCGAGA |
| | ACGGCCTGAAGATCGACATCCACGTGATCATCCCTTACG |
| | AGGGCCTGTCCGGCGATCAGATGGGACAGATCGAGAAG |
| | ATCTTTAAGGTGGTGTACCCCGTGGACGACCACCACTTC |
| | AAAGTGATCCTGCACTACGGCACCCTGGTCATCGATGGC |
| | GTGACCCCTAACATGATCGACTACTTCGGCAGACCCTAC |
| | GAGGGAATCGCCGTGTTCGACGGCAAGAAAATCACCGT |
| | GACCGGCACACTGTGGAACGGCAACAAGATCATCGACG |
| | AGCGGCTGATCAACCCCGATGGCAGCCTGCTGTTCAGAG |
| | TGACCATCAACGGCGTGACAGGATGGCGGCTGTGCGAG |
| | AGAATTCTTGCC |
| Linker | GGTGGTGGTAGCGGAGGTGGAAGTGGCGGAGGCAGTGG |
| | CGGAGGCTCTAGAGGC |
| Pilot peptide | GCGCCCCACTTCCCTGAAATCTCCTTCCCCCCTAAACCC |
| | GATTCTGATTATCAGGCCTTGCTACCATCCGCGCCAGAG |
| | ATCTACTCTCACCTCTCCCCCACCAAACCCGATTACATC |
| | AACCTTCGACCGGCGCCCTAA |

Protein sequence (SEQ ID NO: 46)

| | |
|---|---|
| Src | MGSSKSKPKDPSQRRR |
| Linker | KSRGPGGGSGGGSGGGSGGGSTG |
| NanoLuc | MVFTLEDFVGDWRQTAGYNLDQVLEQGGVSSLFQNLGVS |
| | VTPIQRIVLSGENGLKIDIHVIIPYEGLSGDQMGQIEKIFKVV |
| | YPVDDHHFKVILHYGTLVIDGVTPNMIDYFGRPYEGIAVFD |
| | GKKITVTGTLWNGNKIIDERLINPDGSLLFRVTINGVTGWR |
| | LCERILA |
| Linker | GGGSGGGSGGGSGGGSRG |
| Pilot peptide | APHFPEISFPPKPDSDYQALLPSAPEIYSHLSPTKPDYINLRP |
| | AP |

### Src-Asparaginase II-P

Nucleotide sequence (SEQ ID NO: 47)

| | |
|---|---|
| Src | ATGGGCAGCAGCAAGAGCAAGCCCAAGGATCCCAGCCA |
| | GCGGCGGAGA |
| Linker | AAAAGTAGAGGACCTGGCGGTGGATCTGGCGGAGGAAG |
| | CGGTGGCGGTTCAGGCGGAGGATCTACCGGTATG |
| Asparaginase II | GAATTCTTCAAGAAAACAGCCCTGGCCGCTCTGGTCATG |
| | GGCTTTTCTGGTGCTGCTCTGGCCCTGCCTAACATCACCA |
| | TTCTGGCTACCGGCGGCACAATAGCTGGCGGCGGAGATT |
| | CTGCCACCAAGAGCAATTACACCGTGGGCAAAGTGGGC |
| | GTCGAGAACCTGGTTAATGCCGTGCCTCAGCTGAAGGAC |
| | ATTGCCAACGTGAAGGGCGAGCAGGTCGTGAACATCGG |
| | CAGCCAGGACATGAACGACAACGTGTGGCTGACCCTGG |
| | CTAAGAAGATCAACACCGACTGCGACAAGACCGACGGC |
| | TTCGTGATCACCCACGGCACCGACACCATGGAAGAGAC |
| | AGCCTACTTCCTGGACCTGACCGTGAAGTGCGACAAGCC |
| | CGTGGTTATGGTCGGAGCCATGAGGCCTAGCACCAGCAT |
| | GTCTGCCGACGGACCCTTCAACCTGTACAACGCCGTGGT |
| | TACAGCCGCCGATAAGGCCTCTGCTAATAGAGGCGTGCT |
| | GGTCGTGATGAACGATACCGTGCTGGACGGCAGGGACG |
| | TGACCAAGACCAATACCACCGACGTGGCAACCTTCAAG |
| | AGCGTGAACTATGGCCCTCTGGGCTACATCCACAACGGC |
| | AAGATCGACTACCAGCGGACCCCTGCCAGAAAGCACAC |
| | CAGCGATACCCCTTTCGACGTGTCCAAGCTGAACGAGCT |
| | GCCTAAAGTGGGCATCGTGTACAACTACGCCAACGCCA |
| | GCGACCTGCCTGCCAAAGCTCTTGTGGATGCCGGCTACG |
| | ACGGAATCGTGTCAGCCGGCGTTGGCAACGGCAATCTGT |
| | ACAAGTCCGTGTTCGACACCCTGGCAACCGCCGCCAAAA |
| | CAGGCACAGCCGTCGTCAGATCTAGCAGAGTGCCTACA |
| | GGCGCCACCACACAGGATGCCGAAGTGGACGATGCCAA |
| | ATACGGCTTTGTGGCCTCCGGCACACTGAACCCTCAGAA |
| | AGCCAGAGTGCTGCTCCAGCTGGCCCTGACACAGACCA |
| | AGGATCCCCAGCAGATTCAGCAGATCTTCAACCAGTAC |
| Linker | GGTGGTGGTAGCGGAGGTGGAAGTGGCGGAGGCAGTGG |
| | CGGAGGCTCTAGAGGC |
| Pilot peptide | GCGCCCCACTTCCCTGAAATCTCCTTCCCCCCTAAACCC |
| | GATTCTGATTATCAGGCCTTGCTACCATCCGCGCCAGAG |
| | ATCTACTCTCACCTCTCCCCCACCAAACCCGATTACATC |
| | AACCTTCGACCGGCGCCCTAA |

Protein sequence (SEQ ID NO: 48)

| | |
|---|---|
| Src | MGSSKSKPKDPSQRRR |
| Linker | KSRGPGGGSGGGSGGGSGGGSTGM |
| Asparaginase II | EFFKKTALAALVMGFSGAALALPNITILATGGTIAGGGDSA |
| | TKSNYTVGKVGVENLVNAVPQLKDIANVKGEQVVNIGSQ |
| | DMNDNVWLTLAKKINTDCDKTDGFVITHGTDTMEETAYF |
| | LDLTVKCDKPVVMVGAMRPSTSMSADGPFNLYNAVVTAA |
| | DKASANRGVLVVMNDTVLDGRDVTKTNTTDVATFKSVN |
| | YGPLGYIHNGKIDYQRTPARKHTSDTPFDVSKLNELPKVGI |
| | VYNYANASDLPAKALVDAGYDGIVSAGVGNGNLYKSVFD |
| | TLATAAKTGTAVVRSSRVPTGATTQDAEVDDAKYGFVAS |
| | GTLNPQKARVLLQLALTQTKDPQQIQQIFNQY |
| Linker | GGGSGGGSGGGSGGGSRG |
| Pilot peptide | APHFPEISFPPKPDSDYQALLPSAPEIYSHLSPTKPDYINLRP |
| | AP |

### Reversible Cargo Proteins

The following sequences correspond to ZeoR-PT2A-SBP-cargo polypeptides described in the EXAMPLES section, with PT2A being self-cleavable upstream of SBP.

### ZeoR-PT2A-SBP-Nanoluc

Nucleotide sequence (SEQ ID NO: 49)

| | |
|---|---|
| ZeoR | ATGGCCAAGCTTACATCTGCTGTGCCTGTGCTGACCGCC |
| | AGAGATGTTGCTGGCGCCGTGGAATTCTGGACCGACAG |
| | ACTGGGCTTCAGCCGGGACTTCGTGGAAGATGATTTTGC |
| | CGGCGTCGTGCGGGACGACGTGACCCTGTTTATTAGCGC |
| | CGTGCAGGACCAGGTGGTGCCCGATAATACTCTGGCCTG |
| | GGTCTGGGTTCGAGGCCTGGATGAACTGTATGCCGAGTG |
| | GAGCGAGGTGGTGTCCACCAACTTCAGAGATGCCAGCG |
| | GACCTGCCATGACCGAGATTGGAGAACAGCCTTGGGGC |
| | AGAGAGTTCGCCCTGAGAGATCCTGCCGGAAACTGCGT |
| | GCACTTCGTGGCCGAAGAACAGGAT |
| Linker | GGCGGAGGTTCTGGCGGAGGAAGCGGTGGCGGATCAGG |
| | CGGAGGATCT |
| PT2A | GCCACAAATTTCAGCCTGCTGAAGCAGGCCGGCGACGT |
| | GGAAGAAAATCCTGGACCT |
| SBP | GGCGGACACGTGGTGGAAGGACTTGCTGGCGAACTGGA |
| | ACAGCTGCGGGCCAGACTGGAACACCATCCTCAGGGAC |
| | AAAGAGAGCCT |
| Linker | GGCGGCGGTAGCGGCGGTGGCAGTGGTGGTGGTAGTGG |
| | CGGCGGATCTACCTGCAGG |
| NanoLuc | ATGGTCTTTACCCTGGAAGATTTCGTCGGCGACTGGCGG |
| | CAGACAGCCGGCTATAATCTGGATCAGGTGCTGGAACA |
| | AGGCGGCGTGTCCAGCCTGTTTCAGAACCTGGGAGTGTC |
| | CGTGACACCCATCCAGAGAATCGTGCTGAGCGGCGAGA |
| | ACGGCCTGAAGATCGACATCCACGTGATCATCCCTTACG |
| | AGGGCCTGTCCGGCGATCAGATGGGACAGATCGAGAAG |
| | ATCTTTAAGGTGGTGTACCCCGTGGACGACCACCACTTC |
| | AAAGTGATCCTGCACTACGGCACCCTGGTCATCGATGGC |
| | GTGACCCCTAACATGATCGACTACTTCGGCAGACCCTAC |
| | GAGGGAATCGCCGTGTTCGACGGCAAGAAAATCACCGT |
| | GACCGGCACACTGTGGAACGGCAACAAGATCATCGACG |
| | AGCGGCTGATCAACCCCGATGGCTCCCTGCTGTTCAGAG |
| | TGACCATCAACGGCGTTACAGGCTGGCGGCTGTGCGAG |
| | AGAATTCTGGCTTAA |

Protein sequence (SEQ ID NO: 50)

| | |
|---|---|
| ZeoR | MAKLTSAVPVLTARDVAGAVEFWTDRLGFSRDFVEDDFA |
| | GVVRDDVTLFISAVQDQVVPDNTLAWVWVRGLDELYAE |
| | WSEVVSTNFRDASGPAMTEIGEQPWGREFALRDPAGNCVH |
| | FVAEEQD |
| Linker | GGGSGGGSGGGSGGGS |
| PT2A | ATNFSLLKQAGDVEENPGP |
| SBP | GGHVVEGLAGELEQLRARLEHHPQGQREP |
| Linker | GGGSGGGSGGGSGGGSTCR |
| NanoLuc | MVFTLEDFVGDWRQTAGYNLDQVLEQGGVSSLFQNLGVS |
| | VTPIQRIVLSGENGLKIDIHVIIPYEGLSGDQMGQIEKIFKVV |
| | YPVDDHHFKVILHYGTLVIDGVTPNMIDYFGRPYEGIAVFD |
| | GKKITVTGTLWNGNKIIDERLINPDGSLLFRVTINGVTGWR |
| | LCERILA* |

### ZeoR-PT2A-SBP-eIF4G

Nucleotide sequence (SEQ ID NO: 51)

| | |
|---|---|
| ZeoR | ATGGCCAAGCTTACATCTGCTGTGCCTGTGCTGACCGCC |
| | AGAGATGTTGCTGGCGCCGTGGAATTCTGGACCGACAG |
| | ACTGGGCTTCAGCCGGGACTTCGTGGAAGATGATTTTGC |
| | CGGCGTCGTGCGGGACGACGTGACCCTGTTTATTAGCGC |
| | CGTGCAGGACCAGGTGGTGCCCGATAATACTCTGGCCTG |
| | GGTCTGGGTTCGAGGCCTGGATGAACTGTATGCCGAGTG |
| | GAGCGAGGTGGTGTCCACCAACTTCAGAGATGCCAGCG |
| | GACCTGCCATGACCGAGATTGGAGAACAGCCTTGGGGC |
| | AGAGAGTTCGCCCTGAGAGATCCTGCCGGAAACTGCGT |
| | GCACTTCGTGGCCGAAGAACAGGAT |
| Linker | GGCGGAGGTTCTGGCGGAGGAAGCGGTGGCGGATCAGG |
| | CGGAGGATCT |
| PT2A | GCCACAAATTTCAGCCTGCTGAAGCAGGCCGGCGACGT |
| | GGAAGAAAATCCTGGACCT |
| SBP | GGCGGACACGTGGTGGAAGGACTTGCTGGCGAACTGGA |
| | ACAGCTGCGGGCCAGACTGGAACACCATCCTCAGGGAC |
| | AAAGAGAGCCT |
| Linker | GGCGGCGGTAGCGGCGGTGGCAGTGGTGGTGGTAGTGG |
| | CGGCGGATCTACCTGCAGGTTCGCCAGC |
| eIF4G | ATGCAGAAGCCTGAAGGCCTGCCTCACATCAGCGACGT |
| | GGTGCTGGATAAGGCCAACAAGACCCCTCTGAGGCCTCT |
| | GGACCCTACAAGACTGCAGGGCATCAACTGCGGCCCTG |
| | ACTTCACACCCAGCTTCGCCAATCTGGGCAGAACCACAC |
| | TGAGCACAAGAGGCCCTCCAAGAGGTGGACCTGGCGGA |
| | GAACTTCCTAGAGGACCTCAGGCTGGACTGGGCCCTAGA |
| | AGATCTCAGCAGGGCCCCAGAAAAGAGCCCCGGAAGAT |
| | CATTGCCACCGTGCTGATGACCGAGGACATCAAGCTGAA |
| | CAAGGCCGAGAAGGCCTGGAAGCCCAGCAGCAAAAGAA |
| | CAGCCGCCGACAAGGACAGAGGCGAAGAGGATGCCGAT |
| | GGCAGCAAGACCCAGGACCTGTTCAGAAGAGTGCGGAG |
| | CATCCTGAACAAGCTGACCCCTCAGATGTTCCAGCAGCT |
| | GATGAAGCAAGTGACCCAGCTGGCTATCGACACCGAGG |
| | AAAGACTGAAGGGCGTCATCGACCTGATCTTTGAGAAG |
| | GCCATCAGCGAGCCCAACTTCAGCGTGGCCTACGCCAAC |
| | ATGTGCCGGTGTCTGATGGCCCTGAAGGTGCCAACCACC |
| | GAGAAGCCTACCGTGACCGTGAACTTCAGAAAGCTGCT |
| | GCTGAATCGGTGCCAGAAAGAGTTCGAGAAGGACAAGG |
| | ACGACGACGAGGTGTTCGAGAAAAAGCAGAAAGAGATG |
| | GACGAGGCCGCCACCGCCGAAGAAAGAGGCAGACTGAA |
| | AGAGGAACTGGAAGAAGCCAGAGATATCGCCAGAAGGC |
| | GGAGCCTGGGCAACATCAAGTTTATCGGCGAGCTGTTTA |
| | AGCTGAAGATGCTGACAGAGGCCATCATGCACGACTGC |
| | GTGGTCAAGCTGCTGAAGAACCACGACGAGGAATCCCT |
| | GGAATGCCTGTGCAGACTGCTGACCACCATCGGCAAGG |
| | ACCTGGACTTCGAGAAAGCCAAGCCTCGGATGGACCAG |
| | TACTTCAACCAGATGGAAAAGATCATCAAAGAGAAGAA |
| | AACCAGCAGCCGCATCCGGTTCATGCTGCAGGATGTGCT |
| | GGATCTGAGAGGCAGCAACTGGGTGCCCAGAAGAGGCG |
| | ATCAGGGCCCTAAGACCATCGACCAGATCCACAAAGAG |
| | GCCGAGATGGAAGAACACCGCGAGCACATCAAGGTGCA |
| | GCAGCTCATGGCTAAGGGCAGCGACAAGCGTAGAGGCG |
| | GACCTCCTGGACCTCCAATCAGTAGAGGACTGCCCCTGG |
| | TGGATGACGGCGGATGGAATACCGTGCCTATCAGCAAG |
| | GGCAGCAGACCAATCGACACCAGCAGACTGACCAAGAT |
| | CACCAAGCCTGGCAGCATCGACAGCAACAACCAGCTGT |
| | TTGCTCCTGGCGGCAGACTGTCTTGGGGCAAGGGATCTT |
| | CTGGTGGCTCTGGCGCCAAACCTTCTGATGCCGCTTCTG |
| | AAGCTGCCCGGCCTGCCACAAGCACCCTGAATAGATTTT |
| | CAGCCCTGCAGCAGGCCGTGCCTACCGAGAGCACCGAC |
| | AATAGAAGAGTGGTGCAGAGAAGCAGCCTGAGCAGAGA |
| | GAGAGGCGAAAAGGCTGGCGACAGGGGCGACAGACTG |
| | GAAAGAAGTGAAAGAGGCGGCGATAGAGGCGACCGGCT |
| | GGATAGAGCTAGAACCCCTGCCACCAAGCGGAGCTTCA |
| | GCAAAGAGGTCGAGGAACGGTCCAGAGAGCGGCCTAGT |
| | CAACCTGAGGGACTGAGAAAAGCCGCCAGCCTGACTGA |
| | GGACAGAGACAGAGGTAGAGATGCCGTGAAGCGGGAA |
| | GCTGCTCTGCCTCCTGTGTCTCCTCTGAAAGCCGCTCTGA |
| | GCGAGGAAGAACTGGAAAAGAAATCCAAGGCCATTATC |
| | GAGGAATACCTGCACCTGAACGACATGAAGGAAGCCGT |
| | GCAGTGCGTGCAAGAGCTGGCCTCACCTAGCCTGCTGTT |
| | CATCTTTGTGCGGCACGGCGTGGAATCTACCCTGGAAAG |
| | ATCTGCCATTGCCAGAGAACACATGGGCCAGCTGCTCCA |
| | CCAACTGCTGTGTGCCGGACATCTGAGCACAGCCCAGTA |
| | CTACCAGGGCCTGTACGAGATCCTGGAACTGGCCGAGG |
| | ATATGGAAATCGACATCCCTCACGTGTGGCTGTACCTGG |
| | CCGAGCTGGTCACACCAATTCTGCAAGAGGGCGGAGTG |
| | CCTATGGGAGAGCTGTTCAGAGAGATCACAAAGCCCCT |
| | GCGGCCTCTGGGCAAAGCTGCATCTCTGCTGCTCGAGAT |
| | TCTGGGCCTGCTGTGCAAGTCTATGGGCCCCAAGAAAGT |
| | GGGCACCCTTTGGAGAGAAGCCGGCCTGTCTTGGAAAG |
| | AGTTTCTGCCCGAAGGCCAGGACATCGGCGCCTTTGTGG |
| | CCGAGCAGAAGGTCGAGTATACCCTGGGCGAAGAGTCT |
| | GAGGCTCCAGGCCAAAGAGCACTGCCTAGCGAGGAACT |
| | GAACCGGCAGCTGGAAAAACTGCTGAAAGAAGGCAGCA |
| | GCAACCAGAGAGTGTTCGACTGGATCGAGGCCAACCTG |
| | AGCGAACAGCAGATCGTGTCTAACACCCTTGTGCGCGCC |
| | CTGATGACAGCCGTGTGTTACAGCGCCATCATCTTCGAG |
| | ACACCCCTGAGAGTGGATGTGGCCGTGCTGAAGGCCAG |
| | AGCTAAACTGCTGCAGAAATACCTGTGCGACGAACAGA |
| | AAGAGCTGCAGGCCCTGTACGCTCTGCAGGCTCTGGTGG |
| | TTACACTGGAACAGCCTCCAAACCTGCTGAGGATGTTCT |
| | TCGACGCCCTGTATGACGAGGACGTGGTCAAAGAGGAC |
| | GCCTTCTACAGCTGGGAGAGCAGCAAGGATCCTGCCGA |
| | ACAGCAAGGCAAAGGCGTGGCACTGAAGTCCGTGACCG |
| | CCTTCTTCAAGTGGCTGCGGGAAGCCGAGGAAGAGAGC |
| | GACCATAACTGA |

Protein sequence (SEQ ID NO: 52)

| | |
|---|---|
| ZeoR | MAKLTSAVPVLTARDVAGAVEFWTDRLGFSRDFVEDDFA |
| | GVVRDDVTLFISAVQDQVVPDNTLAWVWVRGLDELYAE |
| | WSEVVSTNFRDASGPAMTEIGEQPWGREFALRDPAGNCVH |
| | FVAEEQD |
| Linker | GGGSGGGSGGGSGGGS |
| PT2A | ATNFSLLKQAGDVEENPGP |
| SBP | GGHVVEGLAGELEQLRARLEHHPQGQREP |
| Linker | GGGSGGGSGGGSGGGSTCRFAS |
| eIF4G | MQKPEGLPHISDVVLDKANKTPLRPLDPTRLQGINCGPDFT |
| | PSFANLGRTTLSTRGPPRGGPGGELPRGPQAGLGPRRSQQG |
| | PRKEPRKIIATVLMTEDIKLNKAEKAWKPSSKRTAADKDR |
| | GEEDADGSKTQDLFRRVRSILNKLTPQMFQQLMKQVTQLA |
| | IDTEERLKGVIDLIFEKAISEPNFSVAYANMCRCLMALKVPT |
| | TEKPTVTVNFRKLLLNRCQKEFEKDKDDDEVFEKKQKEM |
| | DEAATAEERGRLKEELEEARDIARRRSLGNIKFIGELFKLK |
| | MLTEAIMHDCVVKLLKNHDEESLECLCRLLTTIGKDLDFE |
| | KAKPRMDQYFNQMEKIIKEKKTSSRIRFMLQDVLDLRGSN |
| | WVPRRGDQGPKTIDQIHKEAEMEEHREHIKVQQLMAKGS |
| | DKRRGGPPGPPISRGLPLVDDGGWNTVPISKGSRPIDTSRLT |
| | KITKPGSIDSNNQLFAPGGRLSWGKGSSGGSGAKPSDAASE |
| | AARPATSTLNRFSALQQAVPTESTDNRRVVQRSSLSRERGE |
| | KAGDRGDRLERSERGGDRGDRLDRARTPATKRSFSKEVEE |
| | RSRERPSQPEGLRKAASLTEDRDRGRDAVKREAALPPVSPL |
| | KAALSEEELEKKSKAIIEEYLHLNDMKEAVQCVQELASPSL |
| | LFIFVRHGVESTLERSAIAREHMGQLLHQLLCAGHLSTAQY |
| | YQGLYEILELAEDMEIDIPHVWLYLAELVTPILQEGGVPMG |
| | ELFREITKPLRPLGKAASLLLEILGLLCKSMGPKKVGTLWR |
| | EAGLSWKEFLPEGQDIGAFVAEQKVEYTLGEESEAPGQRA |
| | LPSEELNRQLEKLLKEGSSNQRVFDWIEANLSEQQIVSNTL |
| | VRALMTAVCYSAIIFETPLRVDVAVLKARAKLLQKYLCDE |
| | QKELQALYALQALVVTLEQPPNLLRMFFDALYDEDVVKE |
| | DAFYSWESSKDPAEQQGKGVALKSVTAFFKWLREAEEESD |
| | HN* |

### ZeoR-PT2A-SBP-Oct4

Nucleotide sequence (SEQ ID NO: 53)

| | |
|---|---|
| ZeoR | ATGGCCAAGCTTACATCTGCTGTGCCTGTGCTGACCGCC |
| | AGAGATGTTGCTGGCGCCGTGGAATTCTGGACCGACAG |
| | ACTGGGCTTCAGCCGGGACTTCGTGGAAGATGATTTTGC |
| | CGGCGTCGTGCGGGACGACGTGACCCTGTTTATTAGCGC |
| | CGTGCAGGACCAGGTGGTGCCCGATAATACTCTGGCCTG |
| | GGTCTGGGTTCGAGGCCTGGATGAACTGTATGCCGAGTG |
| | GAGCGAGGTGGTGTCCACCAACTTCAGAGATGCCAGCG |
| | GACCTGCCATGACCGAGATTGGAGAACAGCCTTGGGGC |
| | AGAGAGTTCGCCCTGAGAGATCCTGCCGGAAACTGCGT |
| | GCACTTCGTGGCCGAAGAACAGGAT |
| Linker | GGCGGAGGTTCTGGCGGAGGAAGCGGTGGCGGATCAGG |
| | CGGAGGATCT |
| PT2A | GCCACAAATTTCAGCCTGCTGAAGCAGGCCGGCGACGT |
| | GGAAGAAAATCCTGGACCT |
| SBP | GGCGGACACGTGGTGGAAGGACTTGCTGGCGAACTGGA |
| | ACAGCTGCGGGCCAGACTGGAACACCATCCTCAGGGAC |
| | AAAGAGAGCCT |
| Linker | GGCGGCGGTAGCGGCGGTGGCAGTGGTGGTGGTAGTGG |
| | CGGCGGATCTACCTGCAGGTTCGCCAGC |
| Oct4 | ATGGCTGGACATCTGGCCTCCGACTTCGCCTTCTCTCCAC |
| | CACCTGGCGGAGGCGGAGATGGACCAGGTGGACCTGAA |
| | CCTGGATGGGTTGACCCTAGAACCTGGCTGAGCTTTCAG |
| | GGACCTCCTGGCGGACCTGGAATTGGACCTGGTGTTGGC |
| | CCTGGCTCTGAAGTGTGGGGAATCCCTCCTTGTCCTCCA |
| | CCTTACGAGTTCTGTGGCGGCATGGCCTACTGTGGCCCT |
| | CAAGTTGGAGTTGGCCTGGTGCCTCAAGGCGGCCTGGAA |
| | ACATCTCAGCCTGAGGGCGAAGCTGGCGTGGGCGTCGA |
| | GTCTAATTCTGATGGCGCCTCTCCTGAGCCTTGCACCGTT |
| | ACACCTGGCGCCGTGAAGCTGGAAAAAGAGAAACTGGA |
| | ACAGAACCCCGAGGAAAGCCAGGACATCAAGGCCCTGC |
| | AGAAAGAGCTGGAACAGTTCGCCAAGCTGCTGAAGCAG |
| | AAGCGGATCACCCTGGGCTACACACAGGCTGATGTGGG |
| | CCTGACACTGGGCGTGCTGTTTGGCAAGGTGTTCAGCCA |
| | GACCACCATCTGTAGATTCGAAGCCCTGCAGCTGAGCTT |
| | CAAGAACATGTGCAAGCTGCGGCCCCTGCTGCAGAAAT |
| | GGGTTGAAGAGGCCGACAACAACGAGAACCTGCAAGAG |
| | ATCTGCAAGGCCGAGACACTGGTGCAGGCCCGGAAGAG |
| | AAAGAGAACCAGCATCGAGAACAGAGTGCGGGGCAACC |
| | TGGAAAACCTGTTCCTGCAGTGCCCCAAGCCTACACTGC |
| | AGCAGATCAGCCACATTGCCCAGCAGCTGGGACTCGAA |
| | AAGGACGTCGTCAGAGTGTGGTTCTGCAACCGGCGGCA |
| | GAAGGGCAAGAGAAGCAGCAGCGATTACGCCCAGAGAG |
| | AGGACTTTGAGGCCGCTGGCAGTCCTTTTTCTGGCGGCC |
| | CTGTGTCCTTTCCTCTGGCTCCTGGACCTCACTTTGGCAC |
| | ACCTGGCTATGGCAGCCCTCACTTCACAGCCCTGTACAG |
| | CAGCGTGCCCTTTCCAGAAGGCGAGGCCTTTCCTCCTGT |
| | GTCCGTGACAACACTGGGCAGCCCTATGCACAGCAACTG |
| | A |

Protein sequence (SEQ ID NO: 54)

| | |
|---|---|
| ZeoR | MAKLTSAVPVLTARDVAGAVEFWTDRLGFSRDFVEDDFA |
| | GVVRDDVTLFISAVQDQVVPDNTLAWVWVRGLDELYAE |
| | WSEVVSTNFRDASGPAMTEIGEQPWGREFALRDPAGNCVH |
| | FVAEEQD |
| Linker | GGGSGGGSGGGSGGGS |
| PT2A | ATNFSLLKQAGDVEENPGP |
| SBP | GGHVVEGLAGELEQLRARLEHHPQGQREP |
| Linker | GGGSGGGSGGGSGGGSTCRFAS |
| Oct4 | MAGHLASDFAFSPPPGGGGDGPGGPEPGWVDPRTWLSFQ |
| | GPPGGPGIGPGVGPGSEVWGIPPCPPPYEFCGGMAYCGPQV |
| | GVGLVPQGGLETSQPEGEAGVGVESNSDGASPEPCTVTPG |
| | AVKLEKEKLEQNPEESQDIKALQKELEQFAKLLKQKRITLG |
| | YTQADVGLTLGVLFGKVFSQTTICRFEALQLSFKNMCKLRP |
| | LLQKWVEEADNNENLQEICKAETLVQARKRKRTSIENRVR |
| | GNLENLFLQCPKPTLQQISHIAQQLGLEKDVVRVWFCNRR |
| | QKGKRSSSDYAQREDFEAAGSPFSGGPVSFPLAPGPHFGTP |
| | GYGSPHFTALYSSVPFPEGEAFPPVSVTTLGSPMHSN* |

### Reversible Cargo Proteins

The following sequences correspond to SBP-cargo or cargo-SBP polypeptides described in the EXAMPLES section.

### SBP-Oct4

Nucleotide sequence (SEQ ID NO: 55)

| | |
|---|---|
| SBP | ATGGGCGGACACGTGGTGGAAGGACTTGCTGGCGAACT |
| | GGAACAGCTGCGGGCCAGACTGGAACACCATCCTCAGG |
| | GACAAAGAGAGCCT |
| Linker | GGCGGCGGTAGCGGCGGTGGCAGTGGTGGTGGTAGTGG |
| | CGGCGGATCT |
| Oct4 | ATGGCTGGACATCTGGCCTCCGACTTCGCCTTCTCTCCAC |
| | CACCTGGCGGAGGCGGAGATGGACCAGGTGGACCTGAA |
| | CCTGGATGGGTTGACCCTAGAACCTGGCTGAGCTTTCAG |
| | GGACCTCCTGGCGGACCTGGAATTGGACCTGGTGTTGGC |
| | CCTGGCTCTGAAGTGTGGGGAATCCCTCCTTGTCCTCCA |
| | CCTTACGAGTTCTGTGGCGGCATGGCCTACTGTGGCCCT |
| | CAAGTTGGAGTTGGCCTGGTGCCTCAAGGCGGCCTGGAA |
| | ACATCTCAGCCTGAGGGCGAAGCTGGCGTGGGCGTCGA |
| | GTCTAATTCTGATGGCGCCTCTCCTGAGCCTTGCACCGTT |
| | ACACCTGGCGCCGTGAAGCTGGAAAAAGAGAAACTGGA |
| | ACAGAACCCCGAGGAAAGCCAGGACATCAAGGCCCTGC |
| | AGAAAGAGCTGGAACAGTTCGCCAAGCTGCTGAAGCAG |
| | AAGCGGATCACCCTGGGCTACACACAGGCTGATGTGGG |
| | CCTGACACTGGGCGTGCTGTTTGGCAAGGTGTTCAGCCA |
| | GACCACCATCTGTAGATTCGAAGCCCTGCAGCTGAGCTT |
| | CAAGAACATGTGCAAGCTGCGGCCCCTGCTGCAGAAAT |
| | GGGTTGAAGAGGCCGACAACAACGAGAACCTGCAAGAG |
| | ATCTGCAAGGCCGAGACACTGGTGCAGGCCCGGAAGAG |
| | AAAGAGAACCAGCATCGAGAACAGAGTGCGGGGCAACC |
| | TGGAAAACCTGTTCCTGCAGTGCCCCAAGCCTACACTGC |
| | AGCAGATCAGCCACATTGCCCAGCAGCTGGGACTCGAA |
| | AAGGACGTCGTCAGAGTGTGGTTCTGCAACCGGCGGCA |
| | GAAGGGCAAGAGAAGCAGCAGCGATTACGCCCAGAGAG |
| | AGGACTTTGAGGCCGCTGGCAGTCCTTTTTCTGGCGGCC |
| | CTGTGTCCTTTCCTCTGGCTCCTGGACCTCACTTTGGCAC |
| | ACCTGGCTATGGCAGCCCTCACTTCACAGCCCTGTACAG |
| | CAGCGTGCCCTTTCCAGAAGGCGAGGCCTTTCCTCCTGT |
| | GTCCGTGACAACACTGGGCAGCCCTATGCACAGCAACTG |
| | A |

### Protein Sequence (SEQ ID NO: 56)

| | |
|---|---|
| SBP | MGGHVVEGLAGELEQLRARLEHHPQGQREP |
| Linker | GGGSGGGSGGGSGGGS |
| Oct4 | MAGHLASDFAFSPPPGGGGDGPGGPEPGWVDPRTWLSFQ |
| | GPPGGPGIGPGVGPGSEVWGIPPCPPPYEFCGGMAYCGPQV |
| | GVGLVPQGGLETSQPEGEAGVGVESNSDGASPEPCTVTPG |
| | AVKLEKEKLEONPEESQDIKALQKELEQFAKLLKQKRITLG |
| | YTQADVGLTLGVLFGKVFSQTTICRFEALQLSFKNMCKLRP |
| | LLQKWVEEADNNENLQEICKAETLVQARKRKRTSIENRVR |
| | GNLENLFLQCPKPTLQQISHIAQQLGLEKDVVRVWFCNRR |
| | QKGKRSSSDYAQREDFEAAGSPFSGGPVSFPLAPGPHFGTP |
| | GYGSPHFTALYSSVPFPEGEAFPPVSVTTLGSPMHSN* |

### OMOMYC-SBP

Nucleotide sequence (SEQ ID NO: 57)

| | |
|---|---|
| OMOMYC | ATGGACTTCTTCCGCGTGGTGGAAAACCAGCAGCCTCCT |
| | GCCACAATGCCCCTGAACGTGTCCTTCACCAACCGGAAC |
| | TACGACCTGGACTACGACAGCGTGCAGCCCTACTTCTAC |
| | TGCGACGAGGAAGAGAACTTCTACCAGCAGCAGCAACA |
| | GAGCGAACTCCAGCCTCCAGCTCCTAGCGAGGACATCTG |
| | GAAGAAGTTCGAGCTGCTGCCCACACCTCCTCTGAGCCC |
| | TAGTAGAAGATCCGGCCTGTGCAGCCCCAGCTATGTGGC |
| | CGTGACACCTTTTAGCCTGCGGGGCGATAATGATGGCGG |
| | CGGAGGCAGCTTTAGCACCGCCGATCAACTGGAAATGG |
| | TCACAGAGCTGCTCGGCGGCGACATGGTCAACCAGAGC |
| | TTCATCTGCGACCCCGACGACGAGACATTCATCAAGAAC |
| | ATCATCATCCAGGACTGCATGTGGAGCGGCTTTAGCGCC |
| | GCTGCCAAGCTGGTGTCTGAGAAGCTGGCCTCTTATCAG |
| | GCCGCCAGAAAGGATAGCGGCAGCCCCAATCCTGCCAG |
| | AGGCCACTCTGTGTGTAGCACCTCCAGCCTGTACCTGCA |
| | AGATCTGTCTGCCGCCGCTTCCGAGTGCATCGATCCTAG |
| | CGTGGTGTTCCCCTATCCTCTGAACGACAGCAGCTCCCC |
| | TAAGAGCTGTGCCAGCCAGGATAGCAGCGCTTTCAGCCC |
| | TAGCAGCGATAGCCTGCTGAGCAGCACAGAGTCTAGCC |
| | CTCAGGGCTCTCCTGAACCTCTGGTGCTGCACGAGGAAA |
| | CCCCTCCAACCACCAGCAGCGACAGCGAGGAAGAACAA |
| | GAGGACGAAGAGGAAATCGACGTCGTCAGCGTGGAAAA |
| | GAGACAGGCCCCTGGCAAGAGAAGCGAGTCTGGCTCTC |
| | CTTCTGCCGGCGGACACTCTAAGCCTCCACATTCTCCAC |
| | TGGTGCTGAAGCGGTGCCACGTGTCCACACACCAGCACA |
| | ATTATGCCGCTCCTCCAAGCACACGGAAGGACTATCCTG |
| | CCGCCAAGAGAGTGAAGCTGGATAGCGTCAGAGTGCTG |
| | CGGCAGATCAGCAACAACCGGAAGTGCACAAGCCCCAG |
| | AAGCTCCGACACCGAGGAAAACGTGAAGCGGAGAACCC |
| | ACAACGTGCTGGAACGGCAGAGAAGAAACGAGCTGAAG |
| | CGCAGCTTCTTCGCCCTGAGAGATCAGATCCCCGAGCTG |
| | GAAAACAACGAGAAGGCCCCTAAGGTGGTCATCCTGAA |
| | GAAGGCCACCGCCTACATCCTGAGCGTGCAGGCCGAAA |
| | CACAGAAGCTGATCTCCGAGATCGACCTGCTGCGGAAG |
| | CAGAACGAGCAGCTGAAGCACAAGCTGGAACAGCTGAG |
| | AAACAGCTGCGCC |
| Linker | GGCGGTGGATCTGGCGGAGGAAGCGGTGGCGGTTCAGG |
| | CGGAGGATCT |
| SBP | GGCGGACACGTGGTGGAAGGACTTGCTGGCGAACTGGA |
| | ACAGCTGCGGGCCAGACTGGAACACCATCCTCAGGGAC |
| | AAAGAGAGCCT |

Protein sequence (SEQ ID NO: 58)

| | |
|---|---|
| OMOMYC | MDFFRVVENQQPPATMPLNVSFTNRNYDLDYDSVQPYFY |
| | CDEEENFYQQQQQSELQPPAPSEDIWKKFELLPTPPLSPSRR |
| | SGLCSPSYVAVTPFSLRGDNDGGGGSFSTADQLEMVTELL |
| | GGDMVNQSFICDPDDETFIKNIIIQDCMWSGFSAAAKLVSE |
| | KLASYQAARKDSGSPNPARGHSVCSTSSLYLQDLSAAASE |
| | CIDPSVVFPYPLNDSSSPKSCASQDSSAFSPSSDSLLSSTESSP |
| | QGSPEPLVLHEETPPTTSSDSEEEQEDEEEIDVVSVEKRQAP |
| | GKRSESGSPSAGGHSKPPHSPLVLKRCHVSTHQHNYAAPPS |
| | TRKDYPAAKRVKLDSVRVLRQISNNRKCTSPRSSDTEENV |
| | KRRTHNVLERQRRNELKRSFFALRDQIPELENNEKAPKVVI |
| | LKKATAYILSVQAETQKLISEIDLLRKQNEQLKHKLEQLRN |
| | SCA |
| Linker | GGGSGGGSGGGSGGGS |
| SBP | GGHVVEGLAGELEQLRARLEHHPQGQREP |

### Asparaginase II-SBP

Nucleotide sequence (SEQ ID NO: 59)

| | |
|---|---|
| Asparaginase | ATGGAATTCTTCAAGAAAACAGCCCTGGCCGCTCTGGTC |
| II | ATGGGCTTTTCTGGTGCTGCTCTGGCCCTGCCTAACATCA |
| | CCATTCTGGCTACCGGCGGCACAATAGCTGGCGGCGGA |
| | GATTCTGCCACCAAGAGCAATTACACCGTGGGCAAAGT |
| | GGGCGTCGAGAACCTGGTTAATGCCGTGCCTCAGCTGAA |
| | GGACATTGCCAACGTGAAGGGCGAGCAGGTCGTGAACA |
| | TCGGCAGCCAGGACATGAACGACAACGTGTGGCTGACC |
| | CTGGCTAAGAAGATCAACACCGACTGCGACAAGACCGA |
| | CGGCTTCGTGATCACCCACGGCACCGACACCATGGAAG |
| | AGACAGCCTACTTCCTGGACCTGACCGTGAAGTGCGACA |
| | AGCCCGTGGTTATGGTCGGAGCCATGAGGCCTAGCACCA |
| | GCATGTCTGCCGACGGACCCTTCAACCTGTACAACGCCG |
| | TGGTTACAGCCGCCGATAAGGCCTCTGCTAATAGAGGCG |
| | TGCTGGTCGTGATGAACGATACCGTGCTGGACGGCAGG |
| | GACGTGACCAAGACCAATACCACCGACGTGGCAACCTT |
| | CAAGAGCGTGAACTATGGCCCTCTGGGCTACATCCACAA |
| | CGGCAAGATCGACTACCAGCGGACCCCTGCCAGAAAGC |
| | ACACCAGCGATACCCCTTTCGACGTGTCCAAGCTGAACG |
| | AGCTGCCTAAAGTGGGCATCGTGTACAACTACGCCAACG |
| | CCAGCGACCTGCCTGCCAAAGCTCTTGTGGATGCCGGCT |
| | ACGACGGAATCGTGTCAGCCGGCGTTGGCAACGGCAAT |
| | CTGTACAAGTCCGTGTTCGACACCCTGGCAACCGCCGCC |
| | AAAACAGGCACAGCCGTCGTCAGATCTAGCAGAGTGCC |
| | TACAGGCGCCACCACACAGGATGCCGAAGTGGACGATG |
| | CCAAATACGGCTTTGTGGCCTCCGGCACACTGAACCCTC |
| | AGAAAGCCAGAGTGCTGCTCCAGCTGGCCCTGACACAG |
| | ACCAAGGATCCCCAGCAGATTCAGCAGATCTTCAACCAG |
| | TAC |
| Linker | GGCGGTGGATCTGGCGGAGGAAGCGGTGGCGGTTCAGG |
| | CGGAGGATCT |
| SBP | GGCGGACACGTGGTGGAAGGACTTGCTGGCGAACTGGA |
| | ACAGCTGCGGGCCAGACTGGAACACCATCCTCAGGGAC |
| | AAAGAGAGCCT |

Protein sequence (SEQ ID NO: 60)

| | |
|---|---|
| Asparaginase | MEFFKKTALAALVMGFSGAALALPNITILATGGTIAGGGDS |
| II | ATKSNYTVGKVGVENLVNAVPQLKDIANVKGEQVVNIGS |
| | QDMNDNVWLTLAKKINTDCDKTDGFVITHGTDTMEETAY |
| | FLDLTVKCDKPVVMVGAMRPSTSMSADGPFNLYNAVVTA |
| | ADKASANRGVLVVMNDTVLDGRDVTKTNTTDVATFKSV |
| | NYGPLGYIHNGKIDYQRTPARKHTSDTPFDVSKLNELPKVG |
| | IVYNYANASDLPAKALVDAGYDGIVSAGVGNGNLYKSVF |
| | DTLATAAKTGTAVVRSSRVPTGATTQDAEVDDAKYGFVA |
| | SGTLNPQKARVLLQLALTQTKDPQQIQQIFNQY |
| Linker | GGGSGGGSGGGSGGGS |
| SBP | GGHVVEGLAGELEQLRARLEHHPQGQREP |

### Oct4-SBP-eGFP

Nucleotide sequence (SEQ ID NO: 61)

| | |
|---|---|
| Oct4 | ATGGCTGGACATCTGGCCTCCGACTTCGCCTTCTCTCCAC |
| | CACCTGGCGGAGGCGGAGATGGACCAGGTGGACCTGAA |
| | CCTGGATGGGTTGACCCTAGAACCTGGCTGAGCTTTCAG |
| | GGACCTCCTGGCGGACCTGGAATTGGACCTGGTGTTGGC |
| | CCTGGCTCTGAAGTGTGGGGAATCCCTCCTTGTCCTCCA |
| | CCTTACGAGTTCTGTGGCGGCATGGCCTACTGTGGCCCT |
| | CAAGTTGGAGTTGGCCTGGTGCCTCAAGGCGGCCTGGAA |
| | ACATCTCAGCCTGAGGGCGAAGCTGGCGTGGGCGTCGA |
| | GTCTAATTCTGATGGCGCCTCTCCTGAGCCTTGCACCGTT |
| | ACACCTGGCGCCGTGAAGCTGGAAAAAGAGAAACTGGA |
| | ACAGAACCCCGAGGAAAGCCAGGACATCAAGGCCCTCC |
| | AGAAAGAGCTGGAACAGTTCGCCAAGCTGCTGAAGCAG |
| | AAGCGGATCACCCTGGGCTACACACAGGCTGATGTGGG |
| | CCTGACACTGGGCGTGCTGTTTGGCAAGGTGTTCAGCCA |
| | GACCACAATCTGTAGATTCGAAGCCCTCCAGCTGAGCTT |
| | CAAGAACATGTGCAAGCTGCGGCCCCTGCTCCAGAAAT |
| | GGGTTGAAGAGGCCGACAACAACGAGAACCTGCAAGAG |
| | ATCTGCAAGGCCGAGACACTGGTGCAGGCCCGGAAGAG |
| | AAAGAGAACCAGCATCGAGAACAGAGTGCGGGGCAACC |
| | TGGAAAACCTGTTCCTGCAATGCCCCAAGCCTACACTCC |
| | AGCAGATCAGCCACATTGCCCAGCAGCTGGGACTCGAA |
| | AAGGACGTCGTCAGAGTGTGGTTCTGCAACCGGCGGCA |
| | GAAGGGCAAGAGAAGCAGCAGCGATTACGCCCAGAGAG |
| | AGGACTTTGAGGCCGCTGGCAGTCCTTTTTCTGGCGGCC |
| | CTGTGTCCTTTCCTCTGGCTCCTGGACCTCACTTTGGCAC |
| | ACCTGGCTATGGCAGCCCTCACTTCACAGCCCTGTACAG |
| | CAGCGTGCCCTTTCCAGAAGGCGAGGCCTTTCCTCCTGT |
| | GTCCGTGACAACACTGGGCAGCCCCATGCACAGCAAT |
| Linker | GGCGGCGGTAGCGGCGGTGGCAGTGGTGGTGGTAGTGG |
| | CGGCGGATCT |
| SBP | GGCGGCCATGTGGTTGAAGGACTTGCCGGCGAACTGGA |
| | ACAGCTGAGAGCCCGGCTTGAGCACCATCCTCAGGGAC |
| | AAAGAGAACCT |
| Linker | GGCGGAGGAAGCGGTGGCGGATCAGGTGGTGGATCTGG |
| | CGGCGGATCT |
| eGFP | ATGGTGTCCAAGGGCGAAGAACTGTTCACCGGCGTGGT |
| | GCCCATTCTGGTGGAACTGGATGGGGATGTGAACGGCC |
| | ACAAGTTCAGCGTTAGCGGAGAAGGCGAAGGCGACGCC |
| | ACATACGGAAAGCTGACCCTGAAGTTCATCTGCACCACC |
| | GGCAAGCTGCCTGTGCCTTGGCCTACACTGGTCACAACC |
| | CTGACATACGGCGTGCAGTGCTTCAGCAGATACCCCGAC |
| | CATATGAAGCAGCACGACTTCTTCAAGAGCGCCATGCCT |
| | GAGGGCTACGTGCAAGAGCGGACCATCTTCTTTAAGGAC |
| | GACGGCAACTACAAGACCAGGGCCGAAGTGAAGTTCGA |
| | GGGCGACACCCTGGTCAACCGGATCGAGCTGAAGGGCA |
| | TCGACTTCAAAGAGGACGGCAACATCCTGGGCCACAAG |
| | CTTGAGTACAACTACAACAGCCACAACGTGTACATCATG |
| | GCCGACAAGCAGAAAAACGGCATCAAAGTGAACTTCAA |
| | GATCCGGCACAACATCGAGGACGGCTCTGTGCAGCTGG |
| | CCGATCACTACCAGCAGAACACACCCATCGGAGATGGC |
| | CCTGTGCTGCTGCCCGATAACCACTACCTGAGCACACAG |
| | AGCGCCCTGAGCAAGGACCCCAACGAGAAGAGGGATCA |
| | CATGGTGCTGCTGGAATTCGTGACCGCCGCTGGCATCAC |
| | ACTCGGCATGGATGAGCTGTACAAG |

Protein sequence (SEQ ID NO: 62)

| | |
|---|---|
| Oct4 | MAGHLASDFAFSPPPGGGGDGPGGPEPGWVDPRTWLSFQ |
| | GPPGGPGIGPGVGPGSEVWGIPPCPPPYEFCGGMAYCGPQV |
| | GVGLVPQGGLETSQPEGEAGVGVESNSDGASPEPCTVTPG |
| | AVKLEKEKLEQNPEESQDIKALQKELEQFAKLLKQKRITLG |
| | YTQADVGLTLGVLFGKVFSQTTICRFEALQLSFKNMCKLRP |
| | LLQKWVEEADNNENLQEICKAETLVQARKRKRTSIENRVR |
| | GNLENLFLQCPKPTLQQISHIAQQLGLEKDVVRVWFCNRR |
| | QKGKRSSSDYAQREDFEAAGSPFSGGPVSFPLAPGPHFGTP |
| | GYGSPHFTALYSSVPFPEGEAFPPVSVTTLGSPMHSN |
| Linker | GGGSGGGSGGGSGGGS |
| SBP | GGHVVEGLAGELEQLRARLEHHPQGQREP |
| Linker | GGGSGGGSGGGSGGGS |
| eGFP | MVSKGEELFTGVVPILVELDGDVNGHKFSVSGEGEGDATY |
| | GKLTLKFICTTGKLPVPWPTLVTTLTYGVQCFSRYPDHMK |
| | QHDFFKSAMPEGYVQERTIFFKDDGNYKTRAEVKFEGDTL |
| | VNRIELKGIDFKEDGNILGHKLEYNYNSHNVYIMADKQKN |
| | GIKVNFKIRHNIEDGSVQLADHYQQNTPIGDGPVLLPDNHY |
| | LSTQSALSKDPNEKRDHMVLLEFVTAAGITLGMDELYK |

### SBP-Oct4-eGFP

Nucleotide sequence (SEQ ID NO: 63)

| | |
|---|---|
| SBP | ATGGGCGGCCATGTGGTTGAAGGACTTGCCGGCGAACT |
| | GGAACAGCTGAGAGCCCGGCTTGAGCACCATCCTCAGG |
| | GACAAAGAGAACCT |
| Linker | GGCGGAGGAAGCGGTGGCGGATCAGGTGGTGGATCTGG |
| | CGGCGGATCT |
| Oct4 | ATGGCTGGACATCTGGCCTCCGACTTCGCCTTCTCTCCAC |
| | CACCTGGCGGAGGCGGAGATGGACCAGGTGGACCTGAA |
| | CCTGGATGGGTTGACCCTAGAACCTGGCTGAGCTTTCAG |
| | GGACCTCCTGGCGGACCTGGAATTGGACCTGGTGTTGGC |
| | CCTGGCTCTGAAGTGTGGGGAATCCCTCCTTGTCCTCCA |
| | CCTTACGAGTTCTGTGGCGGCATGGCCTACTGTGGCCCT |
| | CAAGTTGGAGTTGGCCTGGTGCCTCAAGGCGGCCTGGAA |
| | ACATCTCAGCCTGAGGGCGAAGCTGGCGTGGGCGTCGA |
| | GTCTAATTCTGATGGCGCCTCTCCTGAGCCTTGCACCGTT |
| | ACACCTGGCGCCGTGAAGCTGGAAAAAGAGAAACTGGA |
| | ACAGAACCCCGAGGAAAGCCAGGACATCAAGGCCCTCC |
| | AGAAAGAGCTGGAACAGTTCGCCAAGCTGCTGAAGCAG |
| | AAGCGGATCACCCTGGGCTACACACAGGCTGATGTGGG |
| | CCTGACACTGGGCGTGCTGTTTGGCAAGGTGTTCAGCCA |
| | GACCACCATCTGTAGATTCGAAGCCCTCCAGCTGAGCTT |
| | CAAGAACATGTGCAAGCTGCGGCCCCTGCTCCAGAAAT |
| | GGGTTGAAGAGGCCGACAACAACGAGAACCTGCAAGAG |
| | ATCTGCAAGGCCGAGACACTGGTGCAGGCCCGGAAGAG |
| | AAAGAGAACCAGCATCGAGAACAGAGTGCGGGGCAACC |
| | TGGAAAACCTGTTCCTGCAATGCCCCAAGCCTACACTCC |
| | AGCAGATCAGCCACATTGCCCAGCAGCTGGGACTCGAA |
| | AAGGACGTCGTCAGAGTGTGGTTCTGCAACCGGCGGCA |
| | GAAGGGCAAGAGAAGCAGCAGCGATTACGCCCAGAGAG |
| | AGGACTTTGAGGCCGCTGGCAGTCCTTTTTCTGGCGGCC |
| | CTGTGTCCTTTCCTCTGGCTCCTGGACCTCACTTTGGCAC |
| | ACCTGGCTATGGCAGCCCTCACTTCACAGCCCTGTACAG |
| | CAGCGTGCCCTTTCCAGAAGGCGAGGCCTTTCCTCCTGT |
| | GTCCGTGACAACACTGGGCAGCCCCATGCACAGCAAT |
| Linker | GGCGGCGGTAGCGGCGGTGGCAGTGGTGGTGGTAGTGG |
| | CGGCGGATCT |
| eGFP | ATGGTGTCCAAGGGCGAAGAACTGTTCACCGGCGTGGT |
| | GCCCATTCTGGTGGAACTGGATGGGGATGTGAACGGCC |
| | ACAAGTTCAGCGTTAGCGGAGAAGGCGAAGGCGACGCC |
| | ACATACGGAAAGCTGACCCTGAAGTTCATCTGCACCACC |
| | GGCAAGCTGCCTGTGCCTTGGCCTACACTGGTCACAACC |
| | CTGACATACGGCGTGCAGTGCTTCAGCAGATACCCCGAC |
| | CATATGAAGCAGCACGACTTCTTCAAGAGCGCCATGCCT |
| | GAGGGCTACGTGCAAGAGCGGACCATCTTCTTTAAGGAC |
| | GACGGCAACTACAAGACCAGGGCCGAAGTGAAGTTCGA |
| | GGGCGACACCCTGGTCAACCGGATCGAGCTGAAGGGCA |
| | TCGACTTCAAAGAGGACGGCAACATCCTGGGCCACAAG |
| | CTTGAGTACAACTACAACAGCCACAACGTGTACATCATG |
| | GCCGACAAGCAGAAAAACGGCATCAAAGTGAACTTCAA |
| | GATCCGGCACAACATCGAGGACGGCTCTGTGCAGCTGG |
| | CCGATCACTACCAGCAGAACACACCCATCGGAGATGGC |
| | CCTGTGCTGCTGCCCGATAACCACTACCTGAGCACACAG |
| | AGCGCCCTGAGCAAGGACCCCAACGAGAAGAGGGATCA |
| | CATGGTGCTGCTGGAATTCGTGACCGCCGCTGGCATCAC |
| | ACTCGGCATGGATGAGCTGTACAAG |

Protein sequence (SEQ ID NO: 64)

| | |
|---|---|
| SBP | MGGHVVEGLAGELEQLRARLEHHPQGQREP |
| Linker | GGGSGGGSGGGSGGGS |
| Oct4 | MAGHLASDFAFSPPPGGGGDGPGGPEPGWVDPRTWLSFQ |
| | GPPGGPGIGPGVGPGSEVWGIPPCPPPYEFCGGMAYCGPQV |
| | GVGLVPQGGLETSQPEGEAGVGVESNSDGASPEPCTVTPG |
| | AVKLEKEKLEQNPEESQDIKALQKELEQFAKLLKQKRITLG |
| | YTQADVGLTLGVLFGKVFSQTTICRFEALQLSFKNMCKLRP |
| | LLQKWVEEADNNENLQEICKAETLVQARKRKRTSIENRVR |
| | GNLENLFLQCPKPTLQQISHIAQQLGLEKDVVRVWFCNRR |
| | QKGKRSSSDYAQREDFEAAGSPFSGGPVSFPLAPGPHFGTP |
| | GYGSPHFTALYSSVPFPEGEAFPPVSVTTLGSPMHSN |
| Linker | GGGSGGGSGGGSGGGS |
| eGFP | MVSKGEELFTGVVPILVELDGDVNGHKFSVSGEGEGDATY |
| | GKLTLKFICTTGKLPVPWPTLVTTLTYGVQCFSRYPDHMK |
| | QHDFFKSAMPEGYVQERTIFFKDDGNYKTRAEVKFEGDTL |
| | VNRIELKGIDFKEDGNILGHKLEYNYNSHNVYIMADKQKN |
| | GIKVNFKIRHNIEDGSVQLADHYQQNTPIGDGPVLLPDNHY |
| | LSTQSALSKDPNEKRDHMVLLEFVTAAGITLGMDELYK |

### SBP-eGFP

Nucleotide sequence (SEQ ID NO: 65)

| | |
|---|---|
| SBP | ATGGGCGGCCATGTGGTTGAAGGACTTGCCGGCGAACT |
| | GGAACAGCTGAGAGCCCGGCTTGAGCACCATCCTCAGG |
| | GACAAAGAGAACCT |
| Linker | GGCGGAGGAAGCGGTGGCGGATCAGGTGGTGGATCTGG |
| | CGGCGGATCT |
| eGFP | ATGGTGTCCAAGGGCGAAGAACTGTTCACCGGCGTGGT |
| | GCCCATTCTGGTGGAACTGGATGGGGATGTGAACGGCC |
| | ACAAGTTCAGCGTTAGCGGAGAAGGCGAAGGCGACGCC |
| | ACATACGGAAAGCTGACCCTGAAGTTCATCTGCACCACC |
| | GGCAAGCTGCCTGTGCCTTGGCCTACACTGGTCACAACC |
| | CTGACATACGGCGTGCAGTGCTTCAGCAGATACCCCGAC |
| | CATATGAAGCAGCACGACTTCTTCAAGAGCGCCATGCCT |
| | GAGGGCTACGTGCAAGAGCGGACCATCTTCTTTAAGGAC |
| | GACGGCAACTACAAGACCAGGGCCGAAGTGAAGTTCGA |
| | GGGCGACACCCTGGTCAACCGGATCGAGCTGAAGGGCA |
| | TCGACTTCAAAGAGGACGGCAACATCCTGGGCCACAAG |
| | CTTGAGTACAACTACAACAGCCACAACGTGTACATCATG |
| | GCCGACAAGCAGAAAAACGGCATCAAAGTGAACTTCAA |
| | GATCCGGCACAACATCGAGGACGGCTCTGTGCAGCTGG |
| | CCGATCACTACCAGCAGAACACACCCATCGGAGATGGC |
| | CCTGTGCTGCTGCCCGATAACCACTACCTGAGCACACAG |
| | AGCGCCCTGAGCAAGGACCCCAACGAGAAGAGGGATCA |
| | CATGGTGCTGCTGGAATTCGTGACCGCCGCTGGCATCAC |
| | ACTCGGCATGGATGAGCTGTACAAG |

Protein sequence (SEQ ID NO: 66)

| | |
|---|---|
| SBP | MGGHVVEGLAGELEQLRARLEHHPQGQREP |
| Linker | GGGSGGGSGGGSGGGS |
| eGFP | MVSKGEELFTGVVPILVELDGDVNGHKFSVSGEGEGDATY |
| | GKLTLKFICTTGKLPVPWPTLVTTLTYGVQCFSRYPDHMK |
| | QHDFFKSAMPEGYVQERTIFFKDDGNYKTRAEVKFEGDTL |
| | VNRIELKGIDFKEDGNILGHKLEYNYNSHNVYIMADKQKN |
| | GIKVNFKIRHNIEDGSVQLADHYQQNTPIGDGPVLLPDNHY |
| | LSTQSALSKDPNEKRDHMVLLEFVTAAGITLGMDELYK |

1-16. (canceled)

17. A fusion polypeptide comprising, from N-terminal to C-terminal:
(i) a sub-membrane targeting domain,
(ii) optionally, a linker,
(iii) a protein of interest or a functionally or structurally active fragment thereof,
(iv) optionally, a linker, and
(v) a peptide interacting with the Endosomal Sorting Complexes Required for Transport (ESCRT) cellular machinery.

18. The fusion polypeptide according to claim 17, wherein the sub-membrane targeting domain comprises or consists of an amino acid sequence (M)-G-X₁-X₂-X₃-X₄-X₅, wherein X₁, X₂, X₃ and X₄ independently from each other denote any amino acid residue, X₅ denotes a basic amino acid residue, and (M) denotes an initiator methionine which, when located at the N-terminal extremity of the fusion polypeptide, can be removed in vivo by post-translation processing;
optionally wherein the sub-membrane targeting domain further comprises a basic patch comprising or consisting of several basic amino acid residues.

19. The fusion polypeptide according to claim 17, wherein the sub-membrane targeting domain comprises a myristic acid linked to a glycine residue.

20. The fusion polypeptide according to claim 19, wherein the myristic acid is linked to the glycine residue at position 2 of the amino acid sequence (M)-G-X₁-X₂-X₃-X₄-X₅.

21. The fusion polypeptide according to claim 17, wherein the peptide interacting with the ESCRT cellular machinery comprises an amino acid sequence having one, two or three YxxL and/or DYxxL motif(s) (SEQ ID NO: 14), and one, two, three or four PxxP motif(s).

22. The fusion polypeptide according to claim 17, wherein the peptide interacting with the ESCRT cellular machinery comprises an amino acid sequence having three YxxL and/or DYxxL motifs (SEQ ID NO: 14), and four PxxP motifs.

23. The fusion polypeptide according to claim 17, wherein the peptide interacting with the ESCRT cellular machinery comprises or consists of the amino acid sequence with SEQ ID NO: 38 or a variant thereof.

24. The fusion polypeptide according to claim 17, wherein the protein of interest is a therapeutic protein.

25. The fusion polypeptide according to claim 17, wherein the protein of interest is streptavidin or a fragment thereof, wherein the fragment of streptavidin retains its ability to bind to a streptavidin-binding peptide (SBP) and to biotin.

26. A method of targeting a protein of interest in the lumen of an extracellular vesicle, comprising contacting an extracellular vesicle-producing cell with the fusion polypeptide according to claim 17 or with a nucleic acid encoding said fusion polypeptide.

27. The method according to claim 26, comprising the steps of:
contacting an extracellular vesicle-producing cell with a fusion polypeptide comprising from N-terminal to C-terminal: (i) a sub-membrane targeting domain, (ii) optionally, a linker, (iii) a protein of interest or a functionally or structurally active fragment thereof, (iv) optionally, a linker, and (v) a peptide interacting with the Endosomal Sorting Complexes Required for Transport (ESCRT) cellular machinery, or with a nucleic acid encoding said fusion polypeptide;
culturing the extracellular vesicle-producing cell in a suitable culture medium for a time sufficient to allow extracellular vesicle production; and
recovering the extracellular vesicles produced by the extracellular vesicle-producing cell.

28. A population of extracellular vesicles comprising, in their lumen, the fusion polypeptide according to claim 17.

29. The population of extracellular vesicles according to claim 28, wherein the population of extracellular vesicles is obtainable by a method of targeting a protein of interest in the lumen of an extracellular vesicle, comprising contacting an extracellular vesicle-producing cell with a fusion polypeptide comprising from N-terminal to C-terminal: (i) a sub-membrane targeting domain, (ii) optionally, a linker, (iii) a protein of interest or a functionally or structurally active fragment thereof, (iv) optionally, a linker, and (v) a peptide interacting with the Endosomal Sorting Complexes Required for Transport (ESCRT) cellular machinery, or with a nucleic acid encoding said fusion polypeptide.

30. A method of reversibly targeting a protein of interest in the lumen of an extracellular vesicle, comprising contacting an extracellular vesicle-producing cell with:
the fusion polypeptide according to claim 25 or a nucleic acid encoding said fusion polypeptide, and
a fusion polypeptide comprising (i) a protein of interest or a functionally or structurally active fragment thereof and (ii) a streptavidin-binding peptide (SBP), or a nucleic acid encoding said fusion polypeptide.

31. The method according to claim 30, comprising the steps of:
contacting an extracellular vesicle-producing cell with:
a fusion polypeptide comprising from N-terminal to C-terminal: (i) a sub-membrane targeting domain, (ii) optionally, a linker, (iii) a protein of interest or a functionally or structurally active fragment thereof, wherein the protein of interest is streptavidin or a fragment thereof, wherein the fragment of streptavidin retains its ability to bind to a streptavidin-binding peptide (SBP) and to biotin, (iv) optionally, a linker, and (v) a peptide interacting with the Endosomal Sorting Complexes Required for Transport (ESCRT) cellular machinery, or a nucleic acid encoding said fusion polypeptide, and
the fusion polypeptide comprising (i) the protein of interest or a functionally or structurally active fragment thereof and (ii) the streptavidin-binding peptide (SBP), or the nucleic acid encoding said fusion polypeptide;
culturing the extracellular vesicle-producing cell in a suitable culture medium for a time sufficient to allow extracellular vesicle production; and
recovering the extracellular vesicles produced by the extracellular vesicle-producing cell.

32. The method according to claim 30, wherein the protein of interest is a therapeutic protein.

33. The method according to claim 30, wherein the protein of interest is released from the fusion polypeptide by addition of biotin or a structural analog thereof.

34. The population of extracellular vesicles according to claim 28, comprising, in their lumen, the fusion polypeptide, wherein the protein of interest is streptavidin or a fragment thereof, wherein the fragment of streptavidin retains its ability to bind to a streptavidin-binding peptide (SBP) and to biotin, and further comprising a fusion polypeptide comprising (i) a protein of interest or a functionally or structurally active fragment thereof and (ii) a streptavidin-binding peptide (SBP).

35. A population of extracellular vesicles obtainable by the method according to claim 30, wherein said population of extracellular vesicles comprises, in their lumen, a fusion polypeptide comprising from N-terminal to C-terminal: (i) a sub-membrane targeting domain, (ii) optionally, a linker, (iii) a protein of interest or a functionally or structurally active fragment thereof, wherein the protein of interest is streptavidin or a fragment thereof, wherein the fragment of streptavidin retains its ability to bind to a streptavidin-binding peptide (SBP) and to biotin, (iv) optionally, a linker, and (v) a peptide interacting with the Endosomal Sorting Complexes Required for Transport (ESCRT) cellular machinery; and further comprises a fusion polypeptide comprising (i) a protein of interest or a functionally or structurally active fragment thereof and (ii) a streptavidin-binding peptide (SBP).

36. A method for preventing and/or treating a disease in a subject in need thereof, wherein the method comprises administering to the subject the population of extracellular vesicles according to claim 28, wherein the disease is selected from the group consisting of cancer, genetic lysosomal diseases, diabetes, loss of function diseases, inflammation, infectious diseases, acquired immunodeficiencies, aging, and neurological diseases.
